SEQUENCE LISTING (112 sequences)

SEQ ID NO: 1 - protein, 17 aa, synthetic construct
  VARIANT 1: note = X=K OR R
XSSQSLFNSGNQKNYLT

SEQ ID NO: 2 - protein, 7 aa, synthetic construct
WASTRES

SEQ ID NO: 3 - protein, 9 aa, synthetic construct
QNDYSYPLT

SEQ ID NO: 4 - protein, 5 aa, synthetic construct
DYNMH

SEQ ID NO: 5 - protein, 17 aa, synthetic construct
  VARIANT 12: note = X=N or S
  VARIANT 16: note = X=K or Q
YINPNNGGTSYXQKFXG

SEQ ID NO: 6 - protein, 6 aa, synthetic construct
TRYLAV

SEQ ID NO: 7 - protein, 17 aa, synthetic construct
  VARIANT 1: note = X=K OR R
XSSQSLLNSGNQKNYLT

SEQ ID NO: 8 - protein, 7 aa, synthetic construct
WASTRES

SEQ ID NO: 9 - protein, 9 aa, synthetic construct
QNDYSYPFT

SEQ ID NO: 10 - protein, 5 aa, synthetic construct
SYWMH

SEQ ID NO: 11 - protein, 17 aa, synthetic construct
  VARIANT 12: note = X=N or A
  VARIANT 13: note = X=E or Q
  VARIANT 16: note = X=K or Q
  VARIANT 17: note = X=S or G
MIHPNSGSTNYXXKFXX

SEQ ID NO: 12 - protein, 10 aa, synthetic construct
RYYGSISPDY

SEQ ID NO: 13 - protein, 24 aa, synthetic construct
  VARIANT 3: note = X=V or Q
  VARIANT 12: note = X= S or T
  VARIANT 13: note = X=V or A
  VARIANT 14: note = X=T or S
  VARIANT 15: note = X=A or V
  VARIANT 17: note = X=E or D
  VARIANT 18: note = X=K or R
  VARIANT 21: note = X=M or I
  VARIANT 23: note = X=S or T
DIXMTQSPSSLXXXXGXXVTXSXC

SEQ ID NO: 14 - protein, 15 aa, synthetic construct
  VARIANT 8: note = X=Q or K
  VARIANT 9: note = X=P or A
WYQQKPGXXPKLLIY

SEQ ID NO: 15 - protein, 33 aa, synthetic construct
  VARIANT 4: note = X=D or S
  VARIANT 8: note = X=T or S
  VARIANT 23: note = X=V or L
  VARIANT 25: note = X=A or P
  VARIANT 28: note = X=L or F
  VARIANT 30: note = X=V or T
  VARIANT 32: note = X=F or Y
GVPXDRFXGSGSGTDFTLTISSXQXEDXAXYXC

SEQ ID NO: 16 - protein, 10 aa, synthetic construct
  VARIANT 3: note = X=A or G
  VARIANT 9: note = X=L or I
FGXGTKLEXK

SEQ ID NO: 17 - protein, 30 aa, synthetic construct
  VARIANT 1: note = X=E or Q
  VARIANT 5: note = X=Q or V
  VARIANT 9: note = X=P or A
  VARIANT 11: note = X=L or V
  VARIANT 12: note = X=V or K
  VARIANT 13: note = X=R or K
  VARIANT 20: note = X=M or V
XVQLXQSGXEXXXPGASVKXSCKASGYTFT

SEQ ID NO: 18 - protein, 14 aa, synthetic construct
  VARIANT 3: note = X=K or R
  VARIANT 5: note = X=S or A
  VARIANT 6: note = X=H or P
  VARIANT 8: note = X=K or Q
  VARIANT 9: note = X=S or R
  VARIANT 13: note = X=I or M
WVXQXXGXXLEWXG

SEQ ID NO: 19 - protein, 32 aa, synthetic construct
  VARIANT 1: note = X=K or R
  VARIANT 2: note = X=A or V
  VARIANT 4: note = X=L or I
  VARIANT 6: note = X=V or R
  VARIANT 7: note = X=N or D
  VARIANT 8: note = X=K or T
  VARIANT 10: note = X=S or A
  VARIANT 18: note = X=R or S
  VARIANT 21: note = X=T or R
  VARIANT 25: note = X=S or T
XXTXTXXXSXSTAYMELXSLXSEDXAVYYCVT

SEQ ID NO: 20 - protein, 11 aa, synthetic construct
  VARIANT 3: note = X=T or Q
WGXGTTVTVSS

SEQ ID NO: 21 - protein, 24 aa, synthetic construct
  VARIANT 3: note = X=V or Q
  VARIANT 13: note = X=V or A
  VARIANT 14: note = X=T or S
  VARIANT 16: note = X=A or V
  VARIANT 18: note = X=E or D
  VARIANT 19: note = X=K or R
  VARIANT 22: note = X=M or I
  VARIANT 23: note = X=S or T
DIXMTQSPSSLSXXAXGXXVTXXC

SEQ ID NO: 22 - protein, 15 aa, synthetic construct
  VARIANT 8: note = X=Q or K
  VARIANT 9: note = X=P or A
WYQQKPGXXPKLLIY

SEQ ID NO: 23 - protein, 32 aa, synthetic construct
  VARIANT 4: note = X=D or S
  VARIANT 7: note = X=T or S
  VARIANT 22: note = X=V or L
  VARIANT 24: note = X=A or P
  VARIANT 27: note = X=L or F
  VARIANT 29: note = X=V or T
GVPXRFXGSGSGTDFTLTISSXQXEDXAXYYC

SEQ ID NO: 24 - protein, 10 aa, synthetic construct
  VARIANT 3: note = X=S or G
FGXGTKLEIK

SEQ ID NO: 25 - protein, 30 aa, synthetic construct
  VARIANT 5: note = X=Q or V
  VARIANT 7: note = X=P or S
  VARIANT 9: note = X=S or A
  VARIANT 11: note = X=L or V
  VARIANT 12: note = X=V or K
  VARIANT 20: note = X=L or V
QVQLXQXGXEXXKPGASVKXSCKASGYTFT

SEQ ID NO: 26 - protein, 14 aa, synthetic construct
  VARIANT 3: note = X=K or R
  VARIANT 5: note = X=R or A
  VARIANT 13: note = X=I or M
WVXQXPGQGLEWXG

SEQ ID NO: 27 - protein, 33 aa, synthetic construct
  VARIANT 1: note = X=S or G
  VARIANT 2: note = X=R or K
  VARIANT 3: note = X=A or V
  VARIANT 5: note = X=L or M
  VARIANT 9: note = X=K or T
  VARIANT 11: note = X=S or T
  VARIANT 14: note = X=A or V
  VARIANT 17: note = X=Q or E
  VARIANT 22: note = X=T or R
  VARIANT 26: note = X=S or T
XXXTXTVDXSXSTXYMXLSSLXSEDXAVYYCAR

SEQ ID NO: 28 - protein, 11 aa, synthetic construct
  VARIANT 7: note = X=L or V
WGQGTTXTVSS

SEQ ID NO: 29 - protein, 17 aa, synthetic construct
KSSQSLFNSGNQKNYLT

SEQ ID NO: 30 - protein, 7 aa, synthetic construct
WASTRES

SEQ ID NO: 31 - protein, 9 aa, synthetic construct
QNDYSYPLT

SEQ ID NO: 32 - protein, 5 aa, synthetic construct
DYNMH

SEQ ID NO: 33 - protein, 17 aa, synthetic construct
YINPNNGGTSYNQKFKG

SEQ ID NO: 34 - protein, 6 aa, synthetic construct
TRYLAV

SEQ ID NO: 35 - protein, 17 aa, synthetic construct
KSSQSLLNSGNQKNYLT

SEQ ID NO: 36 - protein, 7 aa, synthetic construct
WASTRES

SEQ ID NO: 37 - protein, 9 aa, synthetic construct
QNDYSYPFT

SEQ ID NO: 38 - protein, 5 aa, synthetic construct
SYWMH

SEQ ID NO: 39 - protein, 17 aa, synthetic construct
MIHPNSGSTNYNEKFKS

SEQ ID NO: 40 - protein, 10 aa, synthetic construct
RYYGSISPDY

SEQ ID NO: 41 - protein, 23 aa, synthetic construct
  VARIANT 3: note = X=V or Q
  VARIANT 13: note = X=V or A
  VARIANT 21: note = X=M or I
DIXMTQSPSSLSXTAGEKVTXSC

SEQ ID NO: 42 - protein, 15 aa, synthetic construct
WYQQKPGQPPKLLIY

SEQ ID NO: 43 - protein, 33 aa, synthetic construct
  VARIANT 4: note = X=D or S
  VARIANT 23: note = X=V or L
  VARIANT 32: note = X=F or Y
GVPXDRFTGSGSGTDFTLTISSXQAEDLAVYXC

SEQ ID NO: 44 - protein, 10 aa, synthetic construct
FGAGTKLELK

SEQ ID NO: 45 - protein, 30 aa, synthetic construct
  VARIANT 1: note = X=E or Q
  VARIANT 20: note = X=M or V
XVQLQQSGPELVRPGASVKXSCKASGYTFT

SEQ ID NO: 46 - protein, 14 aa, synthetic construct
  VARIANT 8: note = X=K or Q
  VARIANT 9: note = X=S or R
  VARIANT 13: note = X=I or M
WVKQSHGXXLEWXG

SEQ ID NO: 47 - protein, 32 aa, synthetic construct
  VARIANT 1: note = X=K or R
  VARIANT 2: note = X=A or V
  VARIANT 4: note = X=L or I
  VARIANT 7: note = X=N or D
  VARIANT 8: note = X=K or T
  VARIANT 10: note = X=S or A
XXTXTVXXSXSTAYMELRSLTSEDSAVYYCVT

SEQ ID NO: 48 - protein, 11 aa, synthetic construct
WGTGTTVTVSS

SEQ ID NO: 49 - protein, 23 aa, synthetic construct
  VARIANT 3: note = X=V or Q
  VARIANT 13: note = X=V or A
  VARIANT 21: note = X=M or I
DIXMTQSPSSLSXTAGEKVTXSC

SEQ ID NO: 50 - protein, 15 aa, synthetic construct
  VARIANT 8: note = X=Q or K
  VARIANT 9: note = X=P or A
WYQQKPGXXPKLLIY

SEQ ID NO: 51 - protein, 32 aa, synthetic construct
  VARIANT 4: note = X=D or S
  VARIANT 22: note = X=V or L
GVPXRFTGSGSGTDFTLTISSXQAEDLAVYYC

SEQ ID NO: 52 - protein, 10 aa, synthetic construct
FGSGTKLEIK

SEQ ID NO: 53 - protein, 30 aa, synthetic construct
  VARIANT 20: note = X=L or V
QVQLQQPGSELVKPGASVKXSCKASGYTFT

SEQ ID NO: 54 - protein, 14 aa, synthetic construct
  VARIANT 5: note = X=R or A
  VARIANT 13: note = X=I or M
WVKQXPGQGLEWXG

SEQ ID NO: 55 - protein, 32 aa, synthetic construct
  VARIANT 1: note = X=R or K
  VARIANT 2: note = X=A or V
  VARIANT 4: note = X=L or M
  VARIANT 8: note = X=K or T
  VARIANT 13: note = X=A or V
XXTXTVDXSSSTXYMQLSSLTSEDSAVYYCAR

SEQ ID NO: 56 - protein, 11 aa, synthetic construct
WGQGTTLTVSS

SEQ ID NO: 57 - protein, 113 aa, synthetic construct
DIVMTQSPSSLSVSVGDRVTMTCKSSQSLFNSGNQKNYLTWYQQKPGKAPKLLIYWASTRESGVPDRFSGSGSGTDFTLTISSVQPEDFATYFCQNDYSYPLTFGGGTKLEIK

SEQ ID NO: 58 - protein, 113 aa, synthetic construct
DIQMTQSPSSLSASVGDRVTMTCKSSQSLFNSGNQKNYLTWYQQKPGKAPKLLIYWASTRESGVPSRFSGSGSGTDFTLTISSLQPEDFATYYCQNDYSYPLTFGGGTKLEIK

SEQ ID NO: 59 - protein, 113 aa, synthetic construct
DIQMTQSPSSLSASVGDRVTITCRSSQSLFNSGNQKNYLTWYQQKPGKAPKLLIYWASTRESGVPSRFSGSGSGTDFTLTISSLQPEDFATYYCQNDYSYPLTFGGGTKLEIK

SEQ ID NO: 60 - protein, 115 aa, synthetic construct
EVQLVQSGAEVKKPGASVKMSCKASGYTFTDYNMHWVRQAPGKSLEWIGYINPNNGGTSYNQKFKGKATLTVNKSSSTAYMELSSLRSEDTAVYYCVTTRYLAVWGQGTTVTVSS

SEQ ID NO: 61 - protein, 115 aa, synthetic construct
QVQLVQSGAEVKKPGASVKMSCKASGYTFTDYNMHWVRQAPGQRLEWIGYINPNNGGTSYNQKFKGKATLTVDTSASTAYMELSSLRSEDTAVYYCVTTRYLAVWGQGTTVTVSS

SEQ ID NO: 62 - protein, 115 aa, synthetic construct
QVQLVQSGAEVKKPGASVKVSCKASGYTFTDYNMHWVRQAPGQRLEWIGYINPNNGGTSYNQKFKGRVTITVDTSASTAYMELSSLRSEDTAVYYCVTTRYLAVWGQGTTVTVSS

SEQ ID NO: 63 - protein, 115 aa, synthetic construct
QVQLVQSGAEVKKPGASVKVSCKASGYTFTDYNMHWVRQAPGQRLEWMGYINPNNGGTSYNQKFQGRVTITVDTSASTAYMELSSLRSEDTAVYYCVTTRYLAVWGQGTTVTVSS

SEQ ID NO: 64 - protein, 115 aa, synthetic construct
QVQLVQSGAEVKKPGASVKVSCKASGYTFTDYNMHWVRQAPGQRLEWMGYINPNNGGTSYSQKFQGRVTITRDTSASTAYMELSSLRSEDTAVYYCVTTRYLAVWGQGTTVTVSS

SEQ ID NO: 65 - protein, 113 aa, synthetic construct
DIVMTQSPSSLSVSVGDRVTMTCKSSQSLLNSGNQKNYLTWYQQKPGQPPKLLIYWASTRESGVPDRFSGSGSGTDFTLTISSVQPEDFATYYCQNDYSYPFTFGGGTKLEIK

SEQ ID NO: 66 - protein, 113 aa, synthetic construct
DIQMTQSPSSLSASVGDRVTMTCKSSQSLLNSGNQKNYLTWYQQKPGKAPKLLIYWASTRESGVPSRFSGSGSGTDFTLTISSLQPEDFATYYCQNDYSYPFTFGGGTKLEIK

SEQ ID NO: 67 - protein, 113 aa, synthetic construct
DIQMTQSPSSLSASVGDRVTITCRSSQSLLNSGNQKNYLTWYQQKPGKAPKLLIYWASTRESGVPSRFSGSGSGTDFTLTISSLQPEDFATYYCQNDYSYPFTFGGGTKLEIK

SEQ ID NO: 68 - protein, 119 aa, synthetic construct
QVQLVQSGAEVKKPGASVKLSCKASGYTFTSYWMHWVRQRPGQGLEWIGMIHPNSGSTNYNEKFKSKATLTVDKSTSTAYMELSSLRSEDTAVYYCARRYYGSISPDYWGQGTTVTVSS

SEQ ID NO: 69 - protein, 119 aa, synthetic construct
QVQLVQSGAEVKKPGASVKLSCKASGYTFTSYWMHWVRQAPGQGLEWIGMIHPNSGSTNYNEKFKSRATLTVDTSTSTAYMELSSLRSEDTAVYYCARRYYGSISPDYWGQGTTVTVSS

SEQ ID NO: 70 - protein, 119 aa, synthetic construct
QVQLVQSGAEVKKPGASVKVSCKASGYTFTSYWMHWVRQAPGQGLEWIGMIHPNSGSTNYNEKFKSRVTMTVDTSTSTAYMELSSLRSEDTAVYYCARRYYGSISPDYWGQGTTVTVSS

SEQ ID NO: 71 - protein, 119 aa, synthetic construct
QVQLVQSGAEVKKPGASVKVSCKASGYTFTSYWMHWVRQAPGQGLEWMGMIHPNSGSTNYNEKFKSRVTMTVDTSTSTVYMELSSLRSEDTAVYYCARRYYGSISPDYWGQGTTVTVSS

SEQ ID NO: 72 - protein, 119 aa, synthetic construct
QVQLVQSGAEVKKPGASVKVSCKASGYTFTSYWMHWVRQAPGQGLEWMGMIHPNSGSTNYAQKFQGRVTMTRDTSTSTVYMELSSLRSEDTAVYYCARRYYGSISPDYWGQGTTVTVSS

SEQ ID NO: 73 - protein, 107 aa, synthetic construct [sequence omitted]

SEQ ID NO: 74 - protein, 330 aa, synthetic construct [sequence omitted]

SEQ ID NO: 75 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 220 aa, synthetic construct
DIVMTQSPSSLSVSVGDRVTMTCKSSQSLFNSGNQKNYLTWYQQKPGKAPKLLIYWASTRESGVPDRFSGSGSGTDFTLTISSVQPEDFATYFCQNDYSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 220 aa, synthetic construct
DIQMTQSPSSLSASVGDRVTMTCKSSQSLFNSGNQKNYLTWYQQKPGKAPKLLIYWASTRESGVPSRFSGSGSGTDFTLTISSLQPEDFATYYCQNDYSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 220 aa, synthetic construct
DIQMTQSPSSLSASVGDRVTITCRSSQSLFNSGNQKNYLTWYQQKPGKAPKLLIYWASTRESGVPSRFSGSGSGTDFTLTISSLQPEDFATYYCQNDYSYPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 445 aa, synthetic construct
EVQLVQSGAEVKKPGASVKMSCKASGYTFTDYNMHWVRQAPGKSLEWIGYINPNNGGTSYNQKFKGKATLTVNKSSSTAYMELSSLRSEDTAVYYCVTTRYLAVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 445 aa, synthetic construct
QVQLVQSGAEVKKPGASVKMSCKASGYTFTDYNMHWVRQAPGQRLEWIGYINPNNGGTSYNQKFKGKATLTVDTSASTAYMELSSLRSEDTAVYYCVTTRYLAVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 445 aa, synthetic construct
QVQLVQSGAEVKKPGASVKVSCKASGYTFTDYNMHWVRQAPGQRLEWIGYINPNNGGTSYNQKFKGRVTITVDTSASTAYMELSSLRSEDTAVYYCVTTRYLAVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 445 aa, synthetic construct
QVQLVQSGAEVKKPGASVKVSCKASGYTFTDYNMHWVRQAPGQRLEWMGYINPNNGGTSYNQKFQGRVTITVDTSASTAYMELSSLRSEDTAVYYCVTTRYLAVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 445 aa, synthetic construct
QVQLVQSGAEVKKPGASVKVSCKASGYTFTDYNMHWVRQAPGQRLEWMGYINPNNGGTSYSQKFQGRVTITRDTSASTAYMELSSLRSEDTAVYYCVTTRYLAVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 442 aa, synthetic construct
EVQLVQSGAEVKKPGASVKMSCKASGYTFTDYNMHWVRQAPGKSLEWIGYINPNNGGTSYNQKFKGKATLTVNKSSSTAYMELSSLRSEDTAVYYCVTTRYLAVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 442 aa, synthetic construct
QVQLVQSGAEVKKPGASVKMSCKASGYTFTDYNMHWVRQAPGQRLEWIGYINPNNGGTSYNQKFKGKATLTVDTSASTAYMELSSLRSEDTAVYYCVTTRYLAVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 442 aa, synthetic construct
QVQLVQSGAEVKKPGASVKVSCKASGYTFTDYNMHWVRQAPGQRLEWIGYINPNNGGTSYNQKFKGRVTITVDTSASTAYMELSSLRSEDTAVYYCVTTRYLAVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 442 aa, synthetic construct
QVQLVQSGAEVKKPGASVKVSCKASGYTFTDYNMHWVRQAPGQRLEWMGYINPNNGGTSYNQKFQGRVTITVDTSASTAYMELSSLRSEDTAVYYCVTTRYLAVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - protein, 442 aa, synthetic construct
QVQLVQSGAEVKKPGASVKVSCKASGYTFTDYNMHWVRQAPGQRLEWMGYINPNNGGTSYSQKFQGRVTITRDTSASTAYMELSSLRSEDTAVYYCVTTRYLAVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 220 aa, synthetic construct
DIVMTQSPSSLSVSVGDRVTMTCKSSQSLLNSGNQKNYLTWYQQKPGQPPKLLIYWASTRESGVPDRFSGSGSGTDFTLTISSVQPEDFATYYCQNDYSYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - protein, 220 aa, synthetic construct
DIQMTQSPSSLSASVGDRVTMTCKSSQSLLNSGNQKNYLTWYQQKPGKAPKLLIYWASTRESGVPSRFSGSGSGTDFTLTISSLQPEDFATYYCQNDYSYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 220 aa, synthetic construct
DIQMTQSPSSLSASVGDRVTITCRSSQSLLNSGNQKNYLTWYQQKPGKAPKLLIYWASTRESGVPSRFSGSGSGTDFTLTISSLQPEDFATYYCQNDYSYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 449 aa, synthetic construct
QVQLVQSGAEVKKPGASVKLSCKASGYTFTSYWMHWVRQRPGQGLEWIGMIHPNSGSTNYNEKFKSKATLTVDKSTSTAYMELSSLRSEDTAVYYCARRYYGSISPDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 449 aa, synthetic construct
QVQLVQSGAEVKKPGASVKLSCKASGYTFTSYWMHWVRQAPGQGLEWIGMIHPNSGSTNYNEKFKSRATLTVDTSTSTAYMELSSLRSEDTAVYYCARRYYGSISPDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 449 aa, synthetic construct
QVQLVQSGAEVKKPGASVKVSCKASGYTFTSYWMHWVRQAPGQGLEWIGMIHPNSGSTNYNEKFKSRVTMTVDTSTSTAYMELSSLRSEDTAVYYCARRYYGSISPDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 449 aa, synthetic construct
QVQLVQSGAEVKKPGASVKVSCKASGYTFTSYWMHWVRQAPGQGLEWMGMIHPNSGSTNYNEKFKSRVTMTVDTSTSTVYMELSSLRSEDTAVYYCARRYYGSISPDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 449 aa, synthetic construct
QVQLVQSGAEVKKPGASVKVSCKASGYTFTSYWMHWVRQAPGQGLEWMGMIHPNSGSTNYAQKFQGRVTMTRDTSTSTVYMELSSLRSEDTAVYYCARRYYGSISPDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 446 aa, synthetic construct
QVQLVQSGAEVKKPGASVKLSCKASGYTFTSYWMHWVRQRPGQGLEWIGMIHPNSGSTNYNEKFKSKATLTVDKSTSTAYMELSSLRSEDTAVYYCARRYYGSISPDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 446 aa, synthetic construct
QVQLVQSGAEVKKPGASVKLSCKASGYTFTSYWMHWVRQAPGQGLEWIGMIHPNSGSTNYNEKFKSRATLTVDTSTSTAYMELSSLRSEDTAVYYCARRYYGSISPDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 446 aa, synthetic construct
QVQLVQSGAEVKKPGASVKVSCKASGYTFTSYWMHWVRQAPGQGLEWIGMIHPNSGSTNYNEKFKSRVTMTVDTSTSTAYMELSSLRSEDTAVYYCARRYYGSISPDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 446 aa, synthetic construct
QVQLVQSGAEVKKPGASVKVSCKASGYTFTSYWMHWVRQAPGQGLEWMGMIHPNSGSTNYNEKFKSRVTMTVDTSTSTVYMELSSLRSEDTAVYYCARRYYGSISPDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 446 aa, synthetic construct
QVQLVQSGAEVKKPGASVKVSCKASGYTFTSYWMHWVRQAPGQGLEWMGMIHPNSGSTNYAQKFQGRVTMTRDTSTSTVYMELSSLRSEDTAVYYCARRYYGSISPDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 113 aa, synthetic construct
DIVMTQSPSSLTVTAGEKVTMSCKSSQSLFNSGNQKNYLTWYQQKPGQPPKLLIYWASTRESGVPDRFTGSGSGTDFTLTISSVQAEDLAVYFCQNDYSYPLTFGAGTKLELK

SEQ ID NO: 103 - protein, 115 aa, synthetic construct
EVQLQQSGPELVRPGASVKMSCKASGYTFTDYNMHWVKQSHGKSLEWIGYINPNNGGTSYNQKFKGKATLTVNKSSSTAYMELRSLTSEDSAVYYCVTTRYLAVWGTGTTVTVSS

SEQ ID NO: 104 - protein, 113 aa, synthetic construct
DIVMTQSPSSLSVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIK

SEQ ID NO: 105 - protein, 119 aa, synthetic construct
QVQLQQPGSELVKPGASVKLSCKASGYTFTSYWMHWVKQRPGQGLEWIGMIHPNSGSTNYNEKFKSKATLTVDKSSSTAYMQLSSLTSEDSAVYYCARRYYGSISPDYWGQGTTLTVSS

SEQ ID NO: 106 - protein, 24 aa, synthetic construct
IYIWAPLAGTCGVLLLSLVITLYC

SEQ ID NO: 107 - protein, 24 aa, synthetic construct
FWLPIGCAAFVVVCILGCILICWL

SEQ ID NO: 108 - protein, 26 aa, synthetic construct
VAAILGLGLVLGLLGPLAILLALYLL

SEQ ID NO: 109 - protein, 42 aa, synthetic construct
KRGRKKLLYIFKQPFMRPVQTTQEEDGCSCRFPEEEEGGCEL

SEQ ID NO: 110 - protein, 38 aa, synthetic construct
CWLTKKKYSSSVHDPNGEYMFMRAVNTAKKSRLTDVTL

SEQ ID NO: 111 - protein, 45 aa, synthetic construct
TTTPAPRPPTPAPTIASQPLSLRPEACRPAAGGAVHTRGLDFACD

SEQ ID NO: 112 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2025/0332197 A1
(43) Pub. Date: Oct. 30, 2025

(54) CLAUDIN18.2 HUMANIZED ANTIBODY AND APPLICATION THEREOF

(71) Applicant: SUNSHINE LAKE PHARMA CO., LTD., Dongguan, Guangdong (CN)

(72) Inventors: Junji DONG, Dongguan (CN); Zhiheng REN, Dongguan (CN); Kuo ZHANG, Dongguan (CN); Xiang LI, Dongguan (CN); Shushan LIN, Dongguan (CN); Ju PENG, Dongguan (CN); Lixuan XUE, Dongguan (CN); Linjun ZHOU, Dongguan (CN); Xingfan JIA, Dongguan (CN); Limei HUANG, Dongguan (CN); Kaifeng LI, Dongguan (CN); Guanghui ZHAO, Dongguan (CN); Shiyou CHEN, Dongguan (CN); Xiumei TAN, Dongguan (CN); Wenjia LI, Dongguan (CN)

(73) Assignee: SUNSHINE LAKE PHARMA CO., LTD., Dongguan, Guangdong (CN)

(21) Appl. No.: 18/848,469

(22) PCT Filed: Mar. 17, 2023
(86) PCT No.: PCT/CN2023/082174

§ 371 Date: Sep. 18, 2024

(30) Foreign Application Priority Data
Mar. 18, 2022 (CN) 202210272549.0

(51) Int. Cl. A61K 35/17; A61K 39/00; A61K 40/11; A61K 40/31; A61K 40/42; A61P 35/00; C07K 14/705; C07K 14/725; C07K 16/28

(52) U.S. Cl. CPC A61K 35/17; A61K 40/11; A61K 40/31; A61K 40/4202; A61P 35/00; C07K 14/7051; C07K 14/70517; C07K 14/70532; C07K 14/70578; C07K 16/28; A61K 2039/505; A61K 2239/13; A61K 2239/21; A61K 2239/22; C07K 2317/24; C07K 2317/565; C07K 2317/567

(57) ABSTRACT

A Claudin18.2 humanized antibody and application thereof. The antibody contains a chain variable region (CDR) sequence selected from at least one of the following or an amino acid sequence having at least 90% identity therewith: light chain CDR sequences: SEQ ID NOs: 1-3 and 7-9; and heavy chain CDR sequences: SEQ ID NOs: 4-6 and 10-12; The antibody or an antigen-binding fragment thereof have humanized modification. The humanized antibody can specifically target and bind Claudin18.2, has the same in vitro activity as a human-mouse chimeric anti-Claudin18.2 monoclonal antibody, meets the requirements of druggability, and has significantly prolonged in vivo half-life.

### REFERENCE TO AN ELECTRONIC SEQUENCE LISTING

The present application contains a Sequence Listing that has been submitted electronically and is hereby incorporated by reference in its entirety. The electronic Sequence Listing is named Sequence_Listing_ST26, was created on May 16, 2025, and is 156,026 bytes in size.

### FIELD OF THE INVENTION

The present invention relates to the field of biotechnology. Specifically, the present invention relates to Claudin18.2 humanized antibody and application thereof. More specifically, the present invention relates to antibodies capable of specifically recognizing Claudin18.2 or antigen-binding fragments thereof, nucleic acid molecules, expression vectors, recombinant cells, pharmaceutical compositions, pharmaceutical uses and kits for detecting Claudin18.2.

### BACKGROUND ART

Claudins (CLDN) are a family of proteins whose role is to maintain tight junctions that control the exchange of molecules between cells. It is widely distributed in stomach, pancreas and lung tissues and can be used to diagnose and treat diseases. Claudin18.2 subtype is a gastric-specific subtype and has become an ideal target since it was found that Claudin18.2 is a highly selective molecule and is only widely expressed in cancer cells. Claudin18.2 is usually buried in the gastric mucosa and is basically inaccessible to monoclonal antibodies in normal tissues. The occurrence of malignant tumors can lead to the destruction of tight junctions, which exposing the Claudin18.2 epitopes on the surface of tumor cells and becoming a specific target. Therefore, Claudin18.2 confers specificity to targeted therapy.

At present, there are murine anti-Claudin18.2 monoclonal antibodies prepared by hybridoma technology using immunized mice. However, mouse anti-Claudin18.2 monoclonal antibodies have high immunogenicity when entering the human body, thus affecting the pharmacokinetics in the human body. Therefore, there is still a need to further develop more effective therapeutic antibodies against Claudin18.2.

### SUMMARY OF THE INVENTION

This application is filed based on the inventor's discovery of the following issues and facts:

Claudins are widely distributed in the stomach, pancreas and lung tissues. When malignant tumors occur, the Claudin18.2 epitope on the surface of tumor cells is exposed. Therefore, Claudin18.2 can be used as an effective target for the development of new therapeutic drugs. Currently, there are murine Claudin18.2 monoclonal antibodies, but they have high immunogenicity; therefore, the inventors of the present application replaced the framework region (FR region) and constant region of the anti-Claudin18.2 monoclonal antibody with human ones, retained the CDR region of the variable region of the murine anti-Claudin18.2 monoclonal antibody, and obtained a series of anti-Claudin18.2 humanized antibodies. The inventors found that the humanized antibodies obtained in the present application can not only specifically target and bind to Claudin18.2 and have the same binding activity as the chimeric antibodies, but also have a longer half-life in vivo.

Therefore, in the first aspect of the present invention, the present invention provides an antibody or antigen-binding fragment thereof capable of specifically recognizing Claudin18.2. According to an embodiment of the present invention, the antibody comprises at least one CDR sequence selected from the following or an amino acid sequence having at least 90% identity with it: the CDR sequences of light chain variable region: X₁SSQSLFNSGNQKNYLT (SEQ ID NO: 1), WASTRES (SEQ ID NO: 2), QNDYSYPLT (SEQ ID NO: 3), X₄SSQSLLNSGNQKNYLT (SEQ ID NO: 7), WASTRES (SEQ ID NO: 8) and QNDYSYPFT (SEQ ID NO: 9); the CDR sequences of heavy chain variable region: DYNMH (SEQ ID NO: 4), YINPNNGGTSYX₂QKFX₃G (SEQ ID NO: 5), TRYLAV (SEQ ID NO: 6), SYWMH (SEQ ID NO: 10), MIHPNSGSTNYX₅X₆KFX₇X₈ (SEQ ID No: 11) and RYYGSISPDY (SEQ ID NO: 12); wherein, X₁ is K or R, X₂ is N or S, X₃ is K or Q, X₄ is K or R, X₅ is N or A, X₆ is E or Q, X₇ is K or Q, X₈ is S or G, the antibody or antigen-binding fragment thereof has a humanization modification. It should be noted that the “humanization modification” described in the present application refers to changes in amino acids that can reduce the immunogenicity of the antibody or antigen-binding fragment thereof, including amino acid mutations, insertions, deletions, and additions of chemical groups. The above-mentioned antibody according to the embodiment of the present invention can not only specifically target and bind to Claudin18.2, and has the same binding activity as the chimeric antibody, but also has a longer half-life in vivo.

According to an embodiment of the present invention, the aforementioned antibody or antigen-binding fragment may comprise at least one of the following additional technical features:

According to an embodiment of the present invention, the antibody has a light chain variable region with an amino acid sequence as shown in SEQ ID NO: 57-59 or SEQ ID NO: 65-67, and the antibody has a heavy chain variable region with an amino acid sequence as shown in SEQ ID NO: 60-64 or SEQ ID NO: 68-72.

According to an embodiment of the present invention, the antibody comprises at least one of a heavy chain constant region and a light chain constant region, and at least a part of at least one of the heavy chain constant region and the light chain constant region is derived from at least one of a human antibody; the light chain constant region and the heavy chain constant region of the antibody are both derived from a human IgG antibody or a mutant thereof, in addition, the light chain constant region of the antibody is derived from light chain constant region of human Kappa; the heavy chain constant region of the antibody is derived from human IgG4 or IgG1, or a mutant of IgG4 or IgG1. In addition, the full-length sequence of the antibody constant region is shown in SEQ ID NO:73, 74 or 75.

According to an embodiment of the present invention, the antibody has a light chain with sequence as shown in any one of SEQ ID NO: 76-78 and a heavy chain with sequence as shown in any one of SEQ ID NO: 79-88.

According to an embodiment of the present invention, the antibody has a light chain with sequence as shown in any one of SEQ ID NO: 89-91 and a heavy chain with sequence as shown in any one of SEQ ID NO: 92-101.

In the second aspect of the present invention, the present invention provides a nucleic acid molecule. According to an embodiment of the present invention, the nucleic acid molecule encodes the antibody or antigen-binding fragment thereof provided in the first aspect. The antibodies obtained from the nucleic acid molecules of the embodiments of the present invention can not only specifically target and bind to Claudin18.2 with the same binding activity as the chimeric antibody, but also have a longer in vivo half-life and reduce the internalization ratio of the antibody.

In the third aspect of the present invention, the present invention provides an expression vector. According to an embodiment of the present invention, the expression vector carries the nucleic acid molecule described above. After the expression vector according to the embodiment of the present invention is introduced into a suitable recipient cell, it can effectively realize the expression of the aforementioned antibody or antigen-binding fragment thereof that specifically recognizes Claudin18.2 under the mediation of the regulatory system, thereby realizing the mass acquisition of the antibody or antigen-binding fragment in vitro.

In the fourth aspect of the present invention, the present invention provides an immune cell. According to an embodiment of the present invention, the immune cell carries the nucleic acid molecule described above, or expresses the antibody or antigen-binding fragment thereof described above. According to the embodiments of the present invention, the immune cell can express the aforementioned antibody or antigen-binding fragment thereof that specifically recognize Claudin18.2 in large quantities, so as to obtain the antibody or antigen-binding fragment thereof in large quantities in vitro. Furthermore, the immune cell can be reinfused in vivo to treat Claudin18.2-related diseases.

In the fifth aspect of the present invention, the present invention provides a recombinant cell. According to an embodiment of the present invention, the recombinant cell carries the nucleic acid molecule described above, or expresses the antibody or antigen-binding fragment thereof described above. The recombinant cells according to the embodiments of the present invention can be used for the in vitro expression and mass acquisition of the aforementioned antibodies or antigen-binding fragments specifically recognizing Claudin18.2.

In the sixth aspect of the present invention, the present invention provides a CAR-T cell. According to an embodiment of the present invention, the CAR-T cell includes a chimeric antigen receptor, wherein the chimeric antigen receptor includes: an extracellular region, the extracellular region includes a heavy chain variable region and a light chain variable region of a single-chain antibody, wherein the light chain variable region has an amino acid sequence as shown in SEQ ID NO: 57-59 or SEQ ID NO: 65-67, and the heavy chain variable region has an amino acid sequence as shown in SEQ ID NO: 60-64 or SEQ ID NO: 68-72; a transmembrane region, wherein the transmembrane region is connected to the extracellular region and embedded in the cell membrane of the T lymphocyte; and an intracellular region, wherein the intracellular region is connected to the transmembrane region. The CAR-T cell expressing this chimeric antigen receptor can kill tumor cells expressing Claudin18.2.

In the seventh aspect of the present invention, the present invention provides a pharmaceutical composition. According to an embodiment of the present invention, the pharmaceutical composition includes the aforementioned antibody, the nucleic acid molecule, the expression vector, the immune cell, the recombinant cell or the CAR-T cell. As mentioned above, the antibody, as well as the nucleic acid molecule, the expression vector, the immune cell, the recombinant cell or the CAR-T cell that can obtain the antibody after expression can not only specifically target and bind to Claudin18.2 with the same binding activity as the chimeric antibody, but also have a longer in vivo half-life. Therefore, the antibody or expressed antibody contained in the pharmaceutical composition according to the embodiment of the present invention can specifically target and bind to Claudin18.2, exhibit strong specificity, and exert a good targeting effect, thereby realizing the biological effects of other drugs in the pharmaceutical composition, such as the activity inhibition of Claudin18.2 molecules, the killing of cells expressing Claudin18.2 molecules, and the like. In addition, the pharmaceutical composition according to the embodiments of the present invention can play a diagnostic role, relying on the combination of diagnostic reagents and antibody capable of specifically targeting Claudin18.2 proposed in the first aspect of the present invention. This combination plays a role in the diagnosis of areas of the organism with the abnormal expression of Claudin18.2. For instance, by combining with diagnostic nuclides, nanomaterials, etc., it enables the visualization of cells, tissues, and organs in the biological organism that abnormally expressing Claudin18.2, thereby assisting medical workers or scientific researchers to make more accurate judgments of the lesions.

In the eighth aspect of the present invention, the present invention provides the use of the aforementioned antibody, the aforementioned nucleic acid molecule, the aforementioned expression vector, immune cell, recombinant cell, CAR-T cell or the aforementioned pharmaceutical composition in the manufacture of a medicament for the treatment or prevention of Claudin18.2 related diseases.

Or the present invention provides a method for treating or preventing Claudin18.2 related diseases, wherein the method comprises administering the aforementioned antibody, the aforementioned nucleic acid molecule, the aforementioned expression vector, immune cell, recombinant cell, CAR-T cell, or the aforementioned pharmaceutical composition.

Or the present invention provides the aforementioned antibody, the aforementioned nucleic acid molecule, the aforementioned expression vector, immune cell, recombinant cell, CAR-T cell, or the aforementioned pharmaceutical composition for preparing a drug for treating or preventing Claudin18.2 related diseases.

The Claudin18.2 related diseases include: at least one of gastric cancer, esophageal cancer, pancreatic cancer, lung cancer, colon cancer and rectal cancer.

As mentioned above, the antibody, as well as the nucleic acid molecule, the expression vector, the immune cell, the recombinant cell or the CAR-T cell that can obtain the antibody after expression can not only specifically target and bind to Claudin18.2, and have the same binding activity as the chimeric antibody, but also have a longer in vivo half-life. Therefore, drugs containing the above substances can also effectively target and bind to Claudin18.2, have a longer half-life in vivo, and can effectively kill cells expressing Claudin18.2 molecules.

In the ninth aspect of the present invention, the present invention provides a kit for detecting Claudin 18.2. According to an embodiment of the present invention, the kit includes any one of the antibody described above. The aforementioned Claudin18.2 antibodies can specifically target and bind to Claudin18.2. The kit according to the embodiment of the present invention can achieve specific detection of Claudin18.2. For example, when the antibody is bound with a fluorophore, a fluorescent detection device can be used to realize the localization or real-time detection of Claudin18.2; when the antibody is bound with biotin and other markers, the qualitative or quantitative detection of Claudin18.2 can be achieved by color development reagents; the antibody can also be combined with anti-antibody to achieve a sandwich or double-sandwich method, and then achieve signal step-by-step amplification to detect Claudin 18.2.

In the tenth aspect of the present invention, the present invention provides a use of the aforementioned antibody, the aforementioned nucleic acid molecule, the aforementioned expression vector or the aforementioned recombinant cell in the manufacture of a kit for detecting Claudin 18.2 or diagnosing Claudin 18.2 related diseases. According to the embodiment of the present invention, the kit can directly detect the expression level of Claudin18.2, such as high expression, low expression, and no expression, thereby realizing the diagnosis of the disease. It can also be combined with other diagnostic reagents to obtain the status of organisms, tissues, and cells, such as combined with diagnostic nuclides, to visualize the number of cells expressing Claudin18.2 in the body, the size and location of the tissue, etc.

### DESCRIPTION OF THE DRAWINGS

FIG. 1 is a diagram showing the results of non-reduced and reduced SDS-PAGE identification of protein samples 1B6-mVH-HC (IgG1) mVL, 1B6-huVH1-HC (IgG1) huVL1, 1B6-huVH1-HC (IgG1) huVL2, 1B6-huVH2-HC (IgG1) huVL1, 1B6-huVH2-HC (IgG1) huVL2, and 1B6-huVH3-HC (IgG1) huVL1 obtained through a Protein A column according to an embodiment of the present invention, wherein Samples represents samples;

FIG. 2 is a diagram showing the results of non-reduced and reduced SDS-PAGE identification of protein samples 1B6-huVH3-HC (IgG1) huVL2, 1B6-huVH3-HC (IgG1) huVL3, 1B6-huVH4-HC (IgG1) huVL2, 1B6-huVH4-HC (IgG1) huVL3, 1B6-huVH5-HC (IgG1) huVL2 and 1B6-huVH5-HC (IgG1) huVL3 obtained through a Protein A column according to an embodiment of the present invention, wherein Samples represents samples;

FIG. 3 is a diagram showing the results of non-reduced and reduced SDS-PAGE identification of protein samples 1E7-mVH-HC (IgG1) mVL, 1E7-huVH1-HC (IgG1) huVL1, 1E7-huVH1-HC (IgG1) huVL2, 1E7-huVH2-HC (IgG1) huVL1, 1E7-huVH2-HC (IgG1) huVL2 and 1E7-huVH3-HC (IgG1) huVL1 obtained through a Protein A column according to an embodiment of the present invention, wherein Samples represents samples;

FIG. 4 is a diagram showing the results of non-reduced and reduced SDS-PAGE identification of protein samples 1E7-huVH3-HC (IgG1) huVL2, 1E7-huVH3-HC (IgG1) huVL3, 1E7-huVH4-HC (IgG1) huVL2, 1E7-huVH4-HC (IgG1) huVL3, 1E7-huVH5-HC (IgG1) huVL2 and 1E7-huVH5-HC (IgG1) huVL3 obtained through a Protein A column according to an embodiment of the present invention, wherein Samples represents samples;

FIG. 5 is a diagram showing the results of non-reduced and reduced SDS-PAGE identification of protein samples 1B6-mVHmVL, 1B6-huVH1huVL1, 1B6-huVH1huVL2, 1B6-huVH2huVL1, 1B6-huVH2huVL2 and 1B6-huVH3huVL1 obtained through a Protein A column according to an embodiment of the present invention, wherein Samples represents samples;

FIG. 6 is a diagram showing the results of non-reduced and reduced SDS-PAGE identification of protein samples 1B6-mVHmVL, 1B6-huVH1huVL1, 1B6-huVH1huVL2, 1B6-huVH2huVL1, 1B6-huVH2huVL2 and 1B6-huVH3huVL1 obtained through a Protein A column according to an embodiment of the present invention, wherein Samples represents samples;

FIG. 7 is a diagram showing the results of non-reduced and reduced SDS-PAGE identification of protein samples 1E7-mVHmVL, 1E7-huVH1huVL1, 1E7-huVH1huVL2, 1E7-huVH2huVL1, 1E7-huVH2huVL2 and 1E7-huVH3huVL1 obtained through a Protein A column according to an embodiment of the present invention, wherein Samples represents samples;

FIG. 8 is a diagram showing the results of non-reduced and reduced SDS-PAGE identification of protein samples 1E7-huVH3huVL2, 1E7-huVH3huVL3, 1E7-huVH4huVL2, 1E7-huVH4huVL3, 1E7-huVH5huVL2 and 1E7-huVH5huVL3 obtained through a Protein A column according to an embodiment of the present invention, wherein Samples represents samples;

FIG. 9 is an analysis diagram of ELISA test results of humanized antibodies 1B6-huVH1huVL1, 1B6-huVH1huVL2, 1B6-huVH2huVL1, 1B6-huVH2huVL2, 1B6-huVH3huVL1, 1B6-huVH3huVL2, 1B6-hu VH3huVL3, 1B6-hu VH4huVL2, 1B6-huVH4huVL3, 1B6-huVH5huVL2, and 1B6-huVH5huVL3 according to an embodiment of the present invention;

FIG. 10 is an analysis diagram of ELISA test results of humanized antibodies 1E7-huVH1huVL1, 1E7-huVH1huVL2, 1E7-huVH2huVL1, 1E7-huVH2huVL2, 1E7-huVH3huVL1 [symbol] 1E7-huVH3huVL2, 1E7-huVH3huVL3, 1E7-huVH4huVL2, 1E7-huVH4huVL3, 1E7-huVH5huVL2 and 1E7-huVH5huVL3 according to an embodiment of the present invention;

FIG. 11 is an analysis diagram of ELISA test results of humanized antibodies 1E7-huVH1-HC (IgG1) huVL1, 1E7-huVH1-HC (IgG1) huVL2, 1E7-huVH2-HC (IgG1) huVL1, 1E7-huVH2-HC (IgG1) huVL2, 1E7-huVH3-HC (IgG1) huVL1, 1E7-huVH3-HC (IgG1) huVL2, 1E7-huVH3-HC (IgG1) huVL3, 1E7-huVH4-HC (IgG1) huVL2, 1E7-huVH4-HC (IgG1) huVL3, 1E7-huVH5-HC (IgG1) huVL2 and 1E7-huVH5-HC (IgG1) huVL3 according to an embodiment of the present invention;

FIG. 12 is an analysis diagram of ELISA test results of humanized antibodies 1B6-huVH1-HC (IgG1) huVL1, 1B6-huVH1-HC (IgG1) huVL2, 1B6-huVH2-HC (IgG1) huVL1, 1B6-huVH2-HC (IgG1) huVL2, 1B6-huVH3-HC (IgG1) huVL1, 1B6-huVH3-HC (IgG1) huVL2, 1B6-huVH3-HC (IgG1) huVL3, 1B6-huVH4-HC (IgG1) huVL2, 1B6-huVH4-HC (IgG1) huVL3, 1B6-huVH5-HC (IgG1) huVL2 and 1B6-huVH5-HC (IgG1) huVL3 according to an embodiment of the present invention;

FIG. 13 is an analysis diagram of the ADCC activity evaluation results of the chimeric antibody 1B6-mVH-HC (IgG1) mVL, the humanized antibodies 1B6-huVH2-HC (IgG1) huVL1, 1B6-huVH2-HC (IgG1) huVL2, and 1B6-huVH4-HC (IgG1) huVL3 according to an embodiment of the present invention, wherein the abscissa represents the Log value of the antibody concentration (μg/mL), and the ordinate represents the intensity of the luminescent signal;

FIG. 14 is an analysis diagram of the ADCC activity evaluation results of the chimeric antibody 1E7-mVH-HC (IgG1) mVL, the humanized antibodies 1E7-huVH1-HC (IgG1) huVL1, 1E7-huVH1-HC (IgG1) huVL2, 1E7-huVH2-HC (IgG1) huVL1, 1E7-huVH3-HC (IgG1) huVL2 according to an embodiment of the present invention;

FIG. 15 is an analysis diagram of the CDC activity evaluation results of the chimeric antibody 1B6-mVH-HC (IgG1) mVL, the humanized antibody 1B6-huVH2-HC (IgG1) huVL1 and 1B6-huVH3-HC (IgG1) huVL1 according to an embodiment of the present invention, wherein the abscissa represents the Log value of the antibody concentration (μg/mL), and the ordinate represents OD450;

FIG. 16 is an analysis diagram of the CDC activity evaluation results of the chimeric antibody 1E7-mVH-HC (IgG1) mVL, the humanized antibody 1E7-huVH1-HC (IgG1) huVL1, 1E7-huVH1-HC (IgG1) huVL2, 1E7-huVH2-HC (IgG1) huVL1 and 1E7-huVH3-HC (IgG1) huVL2 according to an embodiment of the present invention, wherein the abscissa represents the Log value of the antibody concentration (μg/mL), and the ordinate represents OD450;

FIG. 17 is a diagram showing the internalization detection results of the chimeric antibody according to an embodiment of the present invention, wherein:
17-A represents the internalization detection diagram of the antibodies 1E7-mVH-HC (IgG1) mVL, 1E7-huVH3huVL1, 1E7-huVH3huVL2, 1E7-huVH1huVL1 and 1E7-huVH2huVL2;
17-B represents the internalization detection diagram of the antibodies 1B6-mVH-HC (IgG1) mVL and 1B6-huVH4huVL3;
17-C represents the internalization detection diagram of the antibodies 1B6-mVH-HC (IgG1) mVL, 1B6-huVH3huVL1, 1B6-huVH4-HC (IgG1) huVL3, 1B6-huVH3-HC (IgG1) huVL1, 1B6-mVHmVL and 1B6-huVH3-HC (IgG1) huVL3;

FIG. 18 is a study on the in vitro effects of humanized claudin18.2 antibody CAR-T;

FIG. 19 is a study on the in vivo effects of humanized claudin18.2 antibody CAR-T.

### EXAMPLES

The embodiments of the present invention are described in detail below. Examples of the embodiments are shown in the accompanying drawings, in which the same or similar reference numerals indicate the same or similar elements or elements with the same or similar functions. The embodiments described below with reference to the accompanying drawings are exemplary and are intended to explain the present invention and should not be construed as limiting the present invention.

In the process of describing the present invention, the relevant terms in this document are explained and illustrated. These explanations and illustrations are only for the convenience of understanding the scheme and cannot be regarded as limitations on the protection scheme of the present invention.

### Antibody

As used herein, the term “antibody” is an immunoglobulin molecule capable of binding to a specific antigen. It consists of two light chains with lighter molecular weight and two heavy chains with heavier molecular weight. The heavy (H) and light (L) chains are linked by disulfide bonds to form a tetrapeptide chain molecule. Among them, the amino acid sequence of the amino terminal (N-terminal) of the peptide chain varies greatly, which is called the variable region (V region). The carboxyl terminal (C-terminal) is relatively stable with little change, which is called the constant region (C region). The V regions of the L and H chains are referred to as VL and VH, respectively.

Some regions in the variable region have a higher degree of change in amino acid composition and arrangement order. They are called hypervariable regions (HVR). Hypervariable regions are where antigens and antibodies bind, so they are also called complementarity-determining region (CDR). There are three CDRs on both the heavy and light chain variable regions.

It should be noted that “immunogenicity” refers to the ability to induce an immune response, that is, the ability of an antigen to stimulate specific immune cells, causing them to activate, proliferate, and differentiate, ultimately producing immune effector substances such as antibodies and sensitized lymphocytes. The inventors of the present application further humanized (humanized modification) the screened murine monoclonal antibody sequences to obtain humanized antibodies, which not only retain the affinity and specificity of the parent mouse monoclonal antibody, but also greatly reduce its immunogenicity, improve its safety, and prolong its half-life.

In some embodiments, the invention provides an antibody or antigen-binding fragment capable of specifically recognizing Claudin18.2, wherein the antibody comprises at least one CDR sequence selected from the following or an amino acid sequence having at least 90% identity with it: the CDR sequence of light chain variable region: X₁SSQSLFNSGNQKNYLT (SEQ ID NO: 1), WASTRES (SEQ ID NO: 2), QNDYSYPLT (SEQ ID NO: 3), X₄SSQSLLNSGNQKNYLT (SEQ ID NO: 7), WASTRES (SEQ ID NO: 8) and QNDYSYPFT (SEQ ID NO: 9); the CDR sequence of heavy chain variable region: DYNMH (SEQ ID NO: 4), YINPNNGGTSYX₂QKFX₃G (SEQ ID NO: 5), TRYLAV (SEQ ID NO: 6), SYWMH (SEQ ID NO: 10), MIHPNSGSTNYX₅X₆KFX₇X₈ (SEQ ID No: 11) and RYYGSISPDY (SEQ ID NO: 12); wherein, X₁ is K or R, X₂ is N or S, X₃ is K or Q, X₄ is K or R, X₅ is N or A, X₆ is E or Q, X₇ is K or Q, X₈ is S or G, the antibody or antigen-binding fragment thereof has a humanization modification. It should be noted that the “humanization modification” described in the present application refers to changes in amino acids that can reduce the immunogenicity of the antibody or antigen-binding fragment thereof, including amino acid mutations, insertions, deletions, and additions of chemical groups. The above-mentioned antibody according to the embodiment of the present invention has higher ADCC activity and CDC activity, smaller EC50 and IC50 values, can not only specifically target and bind to Claudin18.2, and has the same binding activity as the chimeric antibody, but also has a longer half-life in vivo. In these embodiments, the antibodies or antigen-binding fragments provided by the present invention have conservative amino acid substitutions. “Antigen-binding fragment” refers to an antibody fragment that retains the ability to specifically bind to an antigen (ROR2). “Conservative amino acid substitution” refers to the replacement of an amino acid with a residue that is biologically, chemically, or structurally similar to another amino acid. Biologically similar means that the substitution does not destroy the biological activity of the Claudin18.2 antibody or the Claudin18.2 antigen. Structural similarity refers to side chains with similar lengths of amino acids, such as alanine, glycine, or serine, or side chains of similar size. Chemical similarity means that amino acids have the same charge or are both hydrophilic or hydrophobic. For example, the hydrophobic residues isoleucine, valine, leucine or methionine are substituted with each other. Alternatively, polar amino acids can be used. For example, lysine is substituted with arginine, aspartic acid is substituted with glutamic acid, asparagine is substituted with glutamine, threonine is substituted with serine, etc.

The “1B6-mVH-HC (IgG1) mVL” described herein can be understood as a chimeric antibody comprising a light chain and a heavy chain, wherein the variable regions of the light chain and the heavy chain are both the variable regions of the murine antibody 1B6, and the constant region is from human IgG1; the “1B6-mVHmVL” described herein can be understood as a chimeric antibody comprising a light chain and a heavy chain, wherein the variable regions of the light chain and the heavy chain are both the variable regions of the murine antibody 1B6, and the constant region is from human IgG4; the “1E7-mVH-HC (IgG1) mVL” described herein can be understood as a chimeric antibody comprising a light chain and a heavy chain, wherein the variable regions of the light chain and the heavy chain are both the variable regions of the murine antibody 1E7, and the heavy chain constant region is from human IgG1; the “1E7-mVHmVL” described herein can be understood as a chimeric antibody comprising a light chain and a heavy chain, wherein the variable regions of the light chain and the heavy chain are both the variable regions of the murine antibody 1E7, and the constant region is from human IgG4; “1B6” and “1E7” can be understood as double-chain antibodies formed by VH-HC-VL-LC.

The naming method of humanized antibodies in this application is “name of murine antibody-name of heavy chain variable region-heavy chain constant region (name of antibody)-light chain variable region”, wherein the light chain constant region is defaulted to the Kappa chain constant region and is not released with a label. When the heavy chain constant region is derived from a human IgG1 antibody, the heavy chain constant region is expressed as HC (IgG1). When the heavy chain constant region is derived from a human IgG4 antibody, the heavy chain constant region is not released. For example, the human antibody “1B6-huVH1-HC (IgG1) huVL1” can be understood as a humanized antibody obtained by replacing the framework region and constant region of the murine antibody 1B6 with the framework region and constant region of the human IgG1 antibody. Preferably, the amino acid sequence of the light chain variable region of the antibody can be shown in SEQ ID NO: 57, the amino acid sequence of the heavy chain variable region of the antibody can be shown in SEQ ID NO: 60, the light chain amino acid sequence of the antibody can be shown in SEQ ID NO: 76, the heavy chain amino acid sequence of the antibody can be shown in SEQ ID NO: 79; the human antibody “1B6-huVH1huVL1” can be understood as a humanized antibody obtained by replacing the framework region and constant region of the murine antibody 1B6 with the framework region and constant region of the human IgG4 antibody. Preferably, the amino acid sequence of the light chain variable region of the antibody can be shown in SEQ ID NO: 57, the amino acid sequence of the heavy chain variable region of the antibody can be shown in SEQ ID NO: 60, the light chain amino acid sequence of the antibody can be shown in SEQ ID NO: 76, the heavy chain amino acid sequence of the antibody can be shown in SEQ ID NO: 84;
the human antibody “1E7-huVH1-HC (IgG1) huVL1” can be understood as a humanized antibody obtained by replacing the framework region and constant region of the murine antibody 1E7 with the framework region and constant region of the human IgG1 antibody. Preferably, the amino acid sequence of the light chain variable region of the antibody can be shown in SEQ ID NO: 65, the amino acid sequence of the heavy chain variable region of the antibody can be shown in SEQ ID NO: 68, the light chain amino acid sequence of the antibody can be shown in SEQ ID NO: 89, the heavy chain amino acid sequence of the antibody can be shown in SEQ ID NO: 92; the human antibody “1E7-huVH1huVL1” can be understood as a humanized antibody obtained by replacing the framework region and constant region of the murine antibody 1E7 with the framework region and constant region of the human IgG4 antibody. Preferably, the amino acid sequence of the light chain variable region of the antibody can be shown in SEQ ID NO: 65, the amino acid sequence of the heavy chain variable region of the antibody can be shown in SEQ ID NO: 68, the light chain amino acid sequence of the antibody can be shown in SEQ ID NO: 89, the heavy chain amino acid sequence of the antibody can be shown in SEQ ID NO: 97.

According to some specific embodiments of the present invention, the aforementioned antibody or antigen-binding fragment may further comprise at least one of the following additional technical features:

According to some specific embodiments of the present invention, the antibody comprises: the light chain variable region CDR1, CDR2, and CDR3 having sequences shown in SEQ ID NO: 1, 2 and 3 respectively or having at least 90% identity with SEQ ID NO: 1, 2 and 3; or the light chain variable region CDR1, CDR2, and CDR3 having sequences shown in SEQ ID NO: 7, 8 and 9 respectively or having at least 90% identity with SEQ ID NO: 7, 8 and 9.

According to some specific embodiments of the present invention, the antibody comprises: the heavy chain variable region CDR1, CDR2, and CDR3 having sequences shown in SEQ ID NO: 4, 5 and 6 respectively or having at least 90% identity with SEQ ID NO: 4, 5 and 6; or the heavy chain variable region CDR1, CDR2, and CDR3 having sequences shown in SEQ ID NO: 10, 11 and 12 respectively or having at least 90% identity with SEQ ID NO: 10, 11 and 12.

According to some specific embodiments of the present invention, the antibody comprises at least one of a heavy chain framework region sequence and a light chain framework region sequence, at least a part of at least one of the heavy chain framework region sequence and the light chain framework region sequence is derived from at least one of a human antibody or a mutant thereof. According to some specific embodiments of the present invention, the inventors performed humanized modification on the light chain framework region and/or the heavy chain framework region of the murine monoclonal antibody.

According to some specific embodiments of the present invention, the antibody comprises: the light chain framework region FRL1, FRL2, FRL3, and FRL4 having sequences shown in SEQ ID NO: 13-16 respectively, or the light chain framework region FRL1, FRL2, FRL3, and FRL4 having sequences shown in SEQ ID NO: 21-24 respectively,

| (SEQ ID NO: 13) | |
|---|---|
| | DIX₉MTQSPSSLX₁₀X₁₁X₁₂X₁₃GX₁₄X₁₅VTX₁₆SX₁₇C, |
| (SEQ ID NO: 14) | |
| | WYQQKPGX₁₈X₁₉PKLLIY, |
| (SEQ ID NO: 15) | |
| | GVPX₂₀DRFX₂₁GSGSGTDFTLTISSX₂₂QX₂₃EDX₂₄AX₂₅YX₂₆C, |
| (SEQ ID NO: 16) | |
| | FGX₂₇GTKLEX₂₈K; |
| (SEQ ID NO: 21) | |
| | DIX₅₃MTQSPSSLSX₅₄X₅₅AX₅₆GX₅₇X₅₈VTX₅₉X₆₀C, |
| (SEQ ID NO: 22) | |
| | WYQQKPGX₆₁X₆₂PKLLIY, |
| (SEQ ID NO: 23) | |
| | GVPX₆₃RFX₆₄GSGSGTDFTLTISSX₆₅QX₆₆EDX₆₇AX₆₈YYC, |
| (SEQ ID NO: 24) | |
| | FGX₆₉GTKLEIK, |

wherein, X₉ is V or Q, X₁₀ is S or T, X₁₁ is V or A, X₁₂ is T or S, X₁₃ is A or V, X₁₄ is E or D, X₁₅ is K or R, X₁₆ is M or I, X₁₇ is S or T, X₁₈ is Q or K, X₁₉ is P or A, X₂₀ is D or S, X₂₁ is T or S, X₂₂ is V or L, X₂₃ is A or P, X₂₄ is L or F, X₂₅ is V or T, X₂₆ is F or Y, X₂₇ is A or G, X₂₈ is L or I, X₅₃ is V or Q, X₅₄ is V or A, X₅₅ is T or S, X₅₆ is A or V, X₅₇ is E or D, X₅₈ is K or R, X₅₉ is M or I, X₆₀ is S or T, X₆₁ is Q or K, X₆₂ is P or A, X₆₃ is D or S, X₆₄ is T or S, X₆₅ is V or L, X₆₆ is A or P, X₆₇ is L or F, X₆₈ is V or T, X₆₉ is S or G. The condition is that when the antibody comprises: the light chain variable region CDR1, CDR2, and CDR3 sequences shown in KSSQSLFNSGNQKNYLT (SEQ ID NO: 29), WASTRES (SEQ ID NO: 30), and QNDYSYPLT (SEQ ID NO: 31), respectively, X₉ is V, X₁₀ is T, X₁₁ is V, X₁₂ is T, X₁₃ is A, X₁₄ is E, X₁₅ is K, X₁₆ is M, X₁₇ is S, X₁₈ is Q, X₁₉ is P, X₂₀ is D, X₂₁ is T, X₂₂ is V, X₂₃ is A, X₂₄ is L, X₂₅ is V, X₂₆ is F, X₂₇ is A, and X₂₈ is L are not established at the same time; when the antibody comprises: the light chain variable region CDR1, CDR2, and CDR3 sequences shown in KSSQSLLNSGNQKNYLT (SEQ ID NO: 35), WASTRES (SEQ ID NO: 36), QNDYSYPFT (SEQ ID NO: 37), respectively, X₅₃ is V, X₅₄ is V, X₅₅ is T, X₅₆ is A, X₅₇ is E, X₅₈ is K, X₅₉ is M, X₆₀ is S, X₆₁ is Q, X₆₂ is P, X₆₃ is D, X₆₄ is T, X₆₅ is V, X₆₆ is A, X₆₇ is L, X₆₈ is V, X₆₉ is S, which are not established at the same time.

According to some specific embodiments of the present invention, the antibody comprises: the light chain framework region FRL1, FRL2, FRL3, and FRL4 having sequences shown in SEQ ID NO: 41-44 respectively, or the light chain framework region FRL1, FRL2, FRL3, and FRL4 having sequences shown in SEQ ID NO: 49-52 respectively,

(SEQ ID NO: 41)
DIX₉₀MTQSPSSLSX₉₁TAGEKVTX₉₂SC,
(SEQ ID NO: 42)
WYQQKPGQPPKLLIY,
(SEQ ID NO: 43)
GVPX₉₃DRFTGSGSGTDFTLTISSX₉₄QAEDLAVYX₉₅C,
(SEQ ID NO: 44)
FGAGTKLELK;

the light chain framework region FRL1, FRL2, FRL3, and FRL4 having sequences shown in SEQ ID NO: 49-52 respectively,

(SEQ ID NO: 49)
DIX₁₀₇MTQSPSSLSX₁₀₈TAGEKVTX₁₀₉SC,
(SEQ ID NO: 50)
WYQQKPGX₁₁₀X₁₁₁PKLLIY,
(SEQ ID NO: 51)
GVPX₁₁₂RFTGSGSGTDFTLTISSX₁₁₃QAEDLAVYYC,
(SEQ ID NO: 52)
FGSGTKLEIK,

wherein, X₉₀ is V or Q, X₉₁ is V or A, X₉₂ is M or I, X₉₃ is D or S, X₉₄ is V or L, X₉₅ is F or Y, X₁₀₇ is V or Q, X₁₀₈ is V or A, X₁₀₉ is M or I, X₁₁₀ is Q or K, X₁₁₁ is P or A, X₁₁₂ is D or S, X₁₁₃ is V or L.

According to some specific embodiments of the present invention, the antibody comprises: the heavy chain framework region FRH1, FRH2, FRH3, and FRH4 having sequences shown in SEQ ID NO: 17-20 respectively, or the heavy chain framework region FRH1, FRH2, FRH3, and FRH4 having sequences shown in SEQ ID NO: 25-28 respectively,

(SEQ ID NO: 17)
X₂₉VQLX₃₀QSGX₃₁EX₃₂X₃₃X₃₄PGASVKX₃₅SCKASGYTFT,
(SEQ ID NO: 18)
WVX₃₆QX₃₇X₃₈GX₃₉X₄₀LEWX₄₁G,
(SEQ ID NO: 19)
X₄₂X₄₃TX₄₄TX₄₅X₄₆X₄₇SX₄₈STAYMELX₄₉SLX₅₀SEDX₅₁AVYYCVT,
(SEQ ID NO: 20)
WGX₅₂GTTVTVSS;
(SEQ ID NO: 25)
QVQLX₇₀QX₇₁GX₇₂EX₇₃X₇₄KPGASVKX₇₅SCKASGYTFT,
(SEQ ID NO: 26)
WVX₇₆QX₇₇PGQGLEWX₇₈G,
(SEQ ID NO: 27)
X₇₉X₈₀X₈₁TX₈₂TVDX₈₃SX₈₄STX₈₅YMX₈₆LSSLX₈₇SEDX₈₈AVYYCAR,
(SEQ ID NO: 28)
WGQGTTX₈₉TVSS,

wherein, X₂₉ is E or Q, X₃₀ is Q or V, X₃₁ is P or A, X₃₂ is L or V, X₃₃ is V or K, X₃₄ is R or K, X₃₅ is M or V, X₃₆ is K or R, X₃₇ is S or A, X₃₈ is H or P, X₃₉ is K or Q, X₄₀ is S or R, X₄₁ is I or M, X₄₂ is K or R, X₄₃ is A or V, X₄₄ is L or I, X₄₅ is V or R, X₄₆ is N or D, X₄₇ is K or T, X₄₈ is S or A, X₄₉ is R or S, X₅₀ is T or R, X₅₁ is S or T, X₅₂ is T or Q, X₇₀ is Q or V, X₇₁ is P or S, X₇₂ is S or A, X₇₃ is L or V, X₇₄ is V or K, X₇₅ is L or V, X₇₆ is K or R, X₇₇ is R or A, X₇₈ is I or M, X₇₉ is S or G, X₈₀ is R or K, X₈₁ is A or V, X₈₂ is L or M, X₈₃ is K or T, X₈₄ is S or T, X₈₅ is A or V, X₈₆ is Q or E, X₈₇ is T or R, X₈₈ is S or T, X₈₉ is L or V; the condition is that when the antibody comprises: the heavy chain variable region CDR1, CDR2, and CDR3 sequences shown in DYNMH (SEQ ID NO: 32), YINPNNGGTSYNQKFKG (SEQ ID NO: 33), TRYLAV (SEQ ID NO: 34), respectively, X₂₉ is E, X₃₀ is Q, X₃₁ is P, X₃₂ is L, X₃₃ is V, X₃₄ is R, X₃₅ is M, X₃₆ is K, X₃₇ is S, X₃₈ is H, X₃₉ is K, X₄₀ is S, X₄₁ is I, X₄₂ is K, X₄₃ is A, X₄₄ is L, X₄₅ is V, X₄₆ is N, X₄₇ is K, X₄₈ is S, X₄₉ is R, X₅₀ is T, X₅₁ is S, X₅₂ is T, which are not established at the same time; when the antibody comprises: the heavy chain variable region CDR1, CDR2, and CDR3 sequences shown in SYWMH (SEQ ID NO: 38), MIHPNSGSTNYNEKFKS (SEQ ID NO: 39), RYYGSISPDY (SEQ ID NO: 40), respectively, X₇₀ is Q, X₇₁ is P, X₇₂ is S, X₇₃ is L, X₇₄ is V, X₇₅ is L, X₇₆ is K, X₇₇ is R, X₇₈ is I, X₇₉ is S, X₈₀ is K, X₈₁ is A, X₈₂ is L, X₈₃ is K, X₈₄ is S, X₈₅ is A, X₈₆ is Q, X₈₇ is T, X₈₈ is S, X₈₉ is L, which are not established at the same time.

According to some specific embodiments of the present invention, the antibody comprises: the heavy chain framework region FRH1, FRH2, FRH3, and FRH4 having sequences shown in SEQ ID NO: 45-48 respectively, or the heavy chain framework region FRH1, FRH2, FRH3, and FRH4 having sequences shown in SEQ ID NO: 53-56 respectively,

(SEQ ID NO: 45)
X₉₆VQLQQSGPELVRPGASVKX₉₇SCKASGYTFT,
(SEQ ID NO: 46)
WVKQSHGX₉₈X₉₉LEWX₁₀₀G,
(SEQ ID NO: 47)
X₁₀₁X₁₀₂TX₁₀₃TVX₁₀₄X₁₀₅SX₁₀₆STAYMELRSLTSEDSAVYYCVT,
(SEQ ID NO: 48)
WGTGTTVTVSS;
(SEQ ID NO: 53)
QVQLQQPGSELVKPGASVKX₁₁₄SCKASGYTFT,
(SEQ ID NO: 54)
WVKQX₁₁₅PGQGLEWX₁₁₆G,
(SEQ ID NO: 55)
X₁₁₇X₁₁₈TX₁₁₉TVDX₁₂₀SSSTX₁₂₁YMQLSSLTSEDSAVYYCAR,
(SEQ ID NO: 56)
WGQGTTLTVSS,

wherein, X₉₆ is E or Q, X₉₇ is M or V, X₉₈ is K or Q, X₉₉ is S or R, X₁₀₀ is I or M, X₁₀₁ is K or R, X₁₀₂ is A or V, X₁₀₃ is L or I, X₁₀₄ is N or D, X₁₀₅ is K or T, X₁₀₆ is S or A, X₁₁₄ is L or V, X₁₁₅ is R or A, X₁₁₆ is I or M, X₁₁₇ is R or K, X₁₁₈ is A or V, X₁₁₉ is L or M, X₁₂₀ is K or T, X₁₂₁ is A or V.

According to some specific embodiments of the present invention, the antibody has a light chain variable region with an amino acid sequence shown in SEQ ID NO: 57-59 or SEQ ID NO: 65-67.

1B6 humanized antibody light chain variable
region 1 (1B6-huVL1):
(SEQ ID NO: 57)
DIVMTQSPSSLSVSVGDRVTMTCKSSQSLFNSGNQKNYLTWYQQKPGKAP
KLLIYWASTRESGVPDRFSGSGSGTDFTLTISSVQPEDFATYFCQNDYSY
PLTFGGGTKLEIK.
1B6 humanized antibody light chain variable
region 2 (1B6-huVL2):
(SEQ ID NO: 58)
DIQMTQSPSSLSASVGDRVTMTCKSSQSLFNSGNQKNYLTWYQQKPGKAP
KLLIYWASTRESGVPSRFSGSGSGTDFTLTISSLQPEDFATYYCQNDYSY
PLTFGGGTKLEIK.
1B6 humanized antibody light chain variable
region 3 (1B6-huVL3):
(SEQ ID NO: 59)
DIQMTQSPSSLSASVGDRVTITCRSSQSLFNSGNQKNYLTWYQQKPGKAP
KLLIYWASTRESGVPSRFSGSGSGTDFTLTISSLQPEDFATYYCQNDYSY
PLTFGGGTKLEIK.
1E7 humanized antibody light chain variable
region 1 (1E7-huVL1):
(SEQ ID NO: 65)
DIVMTQSPSSLSVSVGDRVTMTCKSSQSLLNSGNQKNYLTWYQQKPGQPP
KLLIYWASTRESGVPDRFSGSGSGTDFTLTISSVQPEDFATYYCQNDYSY
PFTFGGGTKLEIK.
1E7 humanized antibody light chain variable
region 2 (1E7-huVL2):
(SEQ ID NO: 66)
DIQMTQSPSSLSASVGDRVTMTCKSSQSLLNSGNQKNYLTWYQQKPGKAP
KLLIYWASTRESGVPSRFSGSGSGTDFTLTISSLQPEDFATYYCQNDYSY
PFTFGGGTKLEIK.
1E7 humanized antibody light chain variable
region 3 (1E7-huVL3):
(SEQ ID NO: 67)
DIQMTQSPSSLSASVGDRVTITCRSSQSLLNSGNQKNYLTWYQQKPGKAP
KLLIYWASTRESGVPSRFSGSGSGTDFTLTISSLQPEDFATYYCQNDYSY
PFTFGGGTKLEIK.

According to some specific embodiments of the present invention, the antibody has a heavy chain variable region with an amino acid sequence shown in SEQ ID NO: 60-64 or SEQ ID NO: 68-72.

1B6 humanized antibody heavy chain variable
region 1 (1B6-huVH1):
(SEQ ID NO: 60)
EVQLVQSGAEVKKPGASVKMSCKASGYTFTDYNMHWVRQAPGKSLEWIGY
INPNNGGTSYNQKFKGKATLTVNKSSSTAYMELSSLRSEDTAVYYCVTTR
YLAVWGQGTTVTVSS.
1B6 humanized antibody heavy chain variable
region 2 (1B6-huVH2):
(SEQ ID NO: 61)
QVQLVQSGAEVKKPGASVKMSCKASGYTFTDYNMHWVRQAPGQRLEWIGY
INPNNGGTSYNQKFKGKATLTVDTSASTAYMELSSLRSEDTAVYYCVTTR
YLAVWGQGTTVTVSS.
1B6 humanized antibody heavy chain variable
region 3 (1B6-huVH3):
(SEQ ID NO: 62)
QVQLVQSGAEVKKPGASVKVSCKASGYTFTDYNMHWVRQAPGQRLEWIGY
INPNNGGTSYNQKFKGRVTITVDTSASTAYMELSSLRSEDTAVYYCVTTR
YLAVWGQGTTVTVSS.
1B6 humanized antibody heavy chain variable
region 4 (1B6-huVH4):
(SEQ ID NO: 63)
QVQLVQSGAEVKKPGASVKVSCKASGYTFTDYNMHWVRQAPGQRLEWMGY
INPNNGGTSYNQKFQGRVTITVDTSASTAYMELSSLRSEDTAVYYCVTTR
YLAVWGQGTTVTVSS.
1B6 humanized antibody heavy chain variable
region 5 (1B6-huVH5):
(SEQ ID NO: 64)
QVQLVQSGAEVKKPGASVKVSCKASGYTFTDYNMHWVRQAPGQRLEWMGY
INPNNGGTSYSQKFQGRVTITRDTSASTAYMELSSLRSEDTAVYYCVTTR
YLAVWGQGTTVTVSS.
1E7 humanized antibody heavy chain variable
region 1 (1E7-huVL1):
(SEQ ID NO: 68)
QVQLVQSGAEVKKPGASVKLSCKASGYTFTSYWMHWVRQRPGQGLEWIGM
IHPNSGSTNYNEKFKSKATLTVDKSTSTAYMELSSLRSEDTAVYYCARRY
YGSISPDYWGQGTTVTVSS.
1E7 humanized antibody heavy chain variable
region 2 (1E7-huVL2):
(SEQ ID NO: 69)
QVQLVQSGAEVKKPGASVKLSCKASGYTFTSYWMHWVRQAPGQGLEWIGM
IHPNSGSTNYNEKFKSRATLTVDTSTSTAYMELSSLRSEDTAVYYCARRY
YGSISPDYWGQGTTVTVSS.
1E7 humanized antibody heavy chain variable
region 3 (1E7-huVL3):
(SEQ ID NO: 70)
QVQLVQSGAEVKKPGASVKVSCKASGYTFTSYWMHWVRQAPGQGLEWIGM
IHPNSGSTNYNEKFKSRVTMTVDTSTSTAYMELSSLRSEDTAVYYCARRY
YGSISPDYWGQGTTVTVSS.
1E7 humanized antibody heavy chain variable
region 4 (1E7-huVL4):
(SEQ ID NO: 71)
QVQLVQSGAEVKKPGASVKVSCKASGYTFTSYWMHWVRQAPGQGLEWMGM
IHPNSGSTNYNEKFKSRVTMTVDTSTSTVYMELSSLRSEDTAVYYCARRY
YGSISPDYWGQGTTVTVSS.
1E7 humanized antibody heavy chain variable
region 5 (1E7-huVL5):
(SEQ ID NO: 72)
QVQLVQSGAEVKKPGASVKVSCKASGYTFTSYWMHWVRQAPGQGLEWMGM
IHPNSGSTNYAQKFQGRVTMTRDTSTSTVYMELSSLRSEDTAVYYCARRY
YGSISPDYWGQGTTVTVSS.

According to some specific embodiments of the present invention, the antibody comprises at least one of a heavy chain constant region and a light chain constant region, and at least a part of at least one of the heavy chain constant region and the light chain constant region is derived from at least one of a human antibody.

According to some specific embodiments of the present invention, both the light chain constant region and the heavy chain constant region of the antibody are derived from a human IgG antibody or a mutant thereof.

According to some specific embodiments of the present invention, the light chain constant region of the antibody is derived from light chain constant region of human Kappa; the heavy chain constant region of the antibody is derived from human IgG4 or IgG1, or a mutant of IgG4 or IgG1.

According to some specific embodiments of the present invention, the full-length sequence of the antibody constant region is shown in SEQ ID NO:73, 74 or 75.

The light chain constant region LC (Kappa):
(SEQ ID NO: 73)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.
The IgG1 heavy chain constant region HC:
(SEQ ID NO: 74)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK.
The IgG4 heavy chain constant region HC:
(SEQ ID NO: 75)
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK.

According to some specific embodiments of the present invention, the antibody has a light chain with an amino acid sequence as shown in any one of SEQ ID NO: 76-78 and a heavy chain with an amino acid sequence as shown in any one of SEQ ID NO: 79-88.

1B6 humanized antibody light chain
1B6-huVL1-LC (Kappa):
(SEQ ID NO: 76)
DIVMTQSPSSLSVSVGDRVTMTCKSSQSLFNSGNQKNYLTWYQQKPGKAP
KLLIYWASTRESGVPDRFSGSGSGTDFTLTISSVQPEDFATYFCQNDYSY
PLTFGGGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC.
1B6 humanized antibody light chain
1B6-huVL2-LC (Kappa):
(SEQ ID NO: 77)
DIQMTQSPSSLSASVGDRVTMTCKSSQSLFNSGNQKNYLTWYQQKPGKAP
KLLIYWASTRESGVPSRFSGSGSGTDFTLTISSLQPEDFATYYCQNDYSY
PLTFGGGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSENRGEC.
1B6 humanized antibody light chain
1B6-huVL3-LC (Kappa):
(SEQ ID NO: 78)
DIQMTQSPSSLSASVGDRVTITCRSSQSLFNSGNQKNYLTWYQQKPGKAP
KLLIYWASTRESGVPSRFSGSGSGTDFTLTISSLQPEDFATYYCQNDYSY
PLTFGGGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC.
1B6 humanized antibody heavy chain
1B6-huVH1-HC (IgG1):
(SEQ ID NO: 79)
EVQLVQSGAEVKKPGASVKMSCKASGYTFTDYNMHWVRQAPGKSLEWIGY
INPNNGGTSYNQKFKGKATLTVNKSSSTAYMELSSLRSEDTAVYYCVTTR
YLAVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLM
ISRTPEVTCVVVDVSHEDPEVKENWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK.
1B6 humanized antibody heavy chain
1B6-huVH2-HC (IgG1):
(SEQ ID NO: 80)
QVQLVQSGAEVKKPGASVKMSCKASGYTFTDYNMHWVRQAPGQRLEWIGY
INPNNGGTSYNQKFKGKATLTVDTSASTAYMELSSLRSEDTAVYYCVTTR
YLAVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK.
1B6 humanized antibody heavy chain
1B6-huVH3-HC (IgG1):
(SEQ ID NO: 81)
QVQLVQSGAEVKKPGASVKVSCKASGYTFTDYNMHWVRQAPGQRLEWIGY
INPNNGGTSYNQKFKGRVTITVDTSASTAYMELSSLRSEDTAVYYCVTTR
YLAVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLM
ISRTPEVTCVVVDVSHEDPEVKENWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK.
1B6 humanized antibody heavy chain
1B6-huVH4-HC (IgG1):
(SEQ ID NO: 82)
QVQLVQSGAEVKKPGASVKVSCKASGYTFTDYNMHWVRQAPGQRLEWMGY
INPNNGGTSYNQKFQGRVTITVDTSASTAYMELSSLRSEDTAVYYCVTTR
YLAVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK.
1B6 humanized antibody heavy chain
1B6-huVH5-HC (IgG1):
(SEQ ID NO: 83)
QVQLVQSGAEVKKPGASVKVSCKASGYTFTDYNMHWVRQAPGQRLEWMGY
INPNNGGTSYSQKFQGRVTITRDTSASTAYMELSSLRSEDTAVYYCVTTR
YLAVWGQGTTVTVSSASTKGPSVFPLAPSSKSTSGGTAALGCLVKDYFPE
PVTVSWNSGALTSGVHTFPAVLQSSGLYSLSSVVTVPSSSLGTQTYICNV
NHKPSNTKVDKKVEPKSCDKTHTCPPCPAPELLGGPSVFLFPPKPKDTLM
ISRTPEVTCVVVDVSHEDPEVKENWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPGK.
1B6 humanized antibody heavy chain
1B6-huVH1-HC (IgG4):
(SEQ ID NO: 84)
EVQLVQSGAEVKKPGASVKMSCKASGYTFTDYNMHWVRQAPGKSLEWIGY
INPNNGGTSYNQKFKGKATLTVNKSSSTAYMELSSLRSEDTAVYYCVTTR
YLAVWGQGTTVTVSSASTKGPSVFPLAPCSRSTSESTAALGCLVKDYFPE
PVTVSWNSGALTSGVHTFPAVLQSSGLYSLSSVVTVPSSSLGTKTYTCNV
DHKPSNTKVDKRVESKYGPPCPPCPAPEFLGGPSVFLFPPKPKDTLMISR
TPEVTCVVVDVSOEDPEVOFNWYVDGVEVHNAKTKPREEQFNSTYRVVSV
LTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFF
LYSRLTVDKSRWQEGNVFSCSVMHEALHNHYTQKSLSLSLGK.
1B6 humanized antibody heavy chain
1B6-huVH2-HC (IgG4):
(SEQ ID NO: 85)
QVQLVQSGAEVKKPGASVKMSCKASGYTFTDYNMHWVRQAPGQRLEWIGY
INPNNGGTSYNQKFKGKATLTVDTSASTAYMELSSLRSEDTAVYYCVTTR
YLAVWGQGTTVTVSSASTKGPSVFPLAPCSRSTSESTAALGCLVKDYFPE
PVTVSWNSGALTSGVHTFPAVLQSSGLYSLSSVVTVPSSSLGTKTYTCNV
DHKPSNTKVDKRVESKYGPPCPPCPAPEFLGGPSVFLFPPKPKDTLMISR
TPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFF
LYSRLTVDKSRWQEGNVFSCSVMHEALHNHYTQKSLSLSLGK.
1B6 humanized antibody heavy chain
1B6-huVH3-HC (IgG4):
(SEQ ID NO: 86)
QVQLVQSGAEVKKPGASVKVSCKASGYTFTDYNMHWVRQAPGQRLEWIGY
INPNNGGTSYNQKFKGRVTITVDTSASTAYMELSSLRSEDTAVYYCVTTR
YLAVWGQGTTVTVSSASTKGPSVFPLAPCSRSTSESTAALGCLVKDYFPE
PVTVSWNSGALTSGVHTFPAVLQSSGLYSLSSVVTVPSSSLGTKTYTCNV
DHKPSNTKVDKRVESKYGPPCPPCPAPEFLGGPSVFLFPPKPKDTLMISR
TPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFF
LYSRLTVDKSRWQEGNVFSCSVMHEALHNHYTQKSLSLSLGK.
1B6 humanized antibody heavy chain
1B6-huVH4-HC (IgG4):
(SEQ ID NO: 87)
QVQLVQSGAEVKKPGASVKVSCKASGYTFTDYNMHWVRQAPGQRLEWMGY
INPNNGGTSYNQKFQGRVTITVDTSASTAYMELSSLRSEDTAVYYCVTTR
YLAVWGQGTTVTVSSASTKGPSVFPLAPCSRSTSESTAALGCLVKDYFPE
PVTVSWNSGALTSGVHTFPAVLQSSGLYSLSSVVTVPSSSLGTKTYTCNV
DHKPSNTKVDKRVESKYGPPCPPCPAPEFLGGPSVFLFPPKPKDTLMISR
TPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFF
LYSRLTVDKSRWQEGNVFSCSVMHEALHNHYTQKSLSLSLGK.
1B6 humanized antibody heavy chain
1B6-huVH5-HC (IgG4):
(SEQ ID NO: 88)
QVQLVQSGAEVKKPGASVKVSCKASGYTFTDYNMHWVRQAPGQRLEWMGY
INPNNGGTSYSQKFQGRVTITRDTSASTAYMELSSLRSEDTAVYYCVTTR
YLAVWGQGTTVTVSSASTKGPSVFPLAPCSRSTSESTAALGCLVKDYFPE
PVTVSWNSGALTSGVHTFPAVLQSSGLYSLSSVVTVPSSSLGTKTYTCNV
DHKPSNTKVDKRVESKYGPPCPPCPAPEFLGGPSVFLFPPKPKDTLMISR
TPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQ
EEMTKNQVSLTCLVKGFYPSDIAVEWESNGQPENNYKTTPPVLDSDGSFF
LYSRLTVDKSRWQEGNVFSCSVMHEALHNHYTQKSLSLSLGK.

According to some specific embodiment of the present invention, the antibody has a light chain with an amino acid sequence as shown in any one of SEQ ID NO: 89-91 and heavy chain with an amino acid sequence as shown in any one of SEQ ID NO: 92-101.

1E7 humanized antibody light chain
1E7-huVL1-LC (Kappa):
(SEQ ID NO: 89)
DIVMTQSPSSLSVSVGDRVTMTCKSSQSLLNSGNQKNYLTWYQQKPGQPP
KLLIYWASTRESGVPDRFSGSGSGTDFTLTISSVQPEDFATYYCQNDYSY
PFTFGGGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC.
1E7 humanized antibody light chain
1E7-huVL2-LC (Kappa):
(SEQ ID NO: 90)
DIQMTQSPSSLSASVGDRVTMTCKSSQSLLNSGNQKNYLTWYQQKPGKAP
KLLIYWASTRESGVPSRFSGSGSGTDFTLTISSLQPEDFATYYCQNDYSY
PFTFGGGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC.
1E7 humanized antibody light chain
1E7-huVL3-LC (Kappa):
(SEQ ID NO: 91)
DIQMTQSPSSLSASVGDRVTITCRSSQSLLNSGNQKNYLTWYQQKPGKAP
KLLIYWASTRESGVPSRFSGSGSGTDFTLTISSLQPEDFATYYCQNDYSY
PFTFGGGTKLEIKRTVAAPSVFIFPPSDEQLKSGTASVVCLLNNFYPREA
KVQWKVDNALQSGNSQESVTEQDSKDSTYSLSSTLTLSKADYEKHKVYAC
EVTHQGLSSPVTKSFNRGEC.
1E7 humanized antibody heavy chain
1E7-huVH1-HC (IgG1):
(SEQ ID NO: 92)
QVQLVQSGAEVKKPGASVKLSCKASGYTFTSYWMHWVRQRPGQGLEWIGM
IHPNSGSTNYNEKFKSKATLTVDKSTSTAYMELSSLRSEDTAVYYCARRY
YGSISPDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK.
1E7 humanized antibody heavy chain
1E7-huVH2-HC (IgG1):
(SEQ ID NO: 93)
QVQLVQSGAEVKKPGASVKLSCKASGYTFTSYWMHWVRQAPGQGLEWIGM
IHPNSGSTNYNEKFKSRATLTVDTSTSTAYMELSSLRSEDTAVYYCARRY
YGSISPDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK.
1E7 humanized antibody heavy chain
1E7-huVH3-HC (IgG1):
(SEQ ID NO: 94)
QVQLVQSGAEVKKPGASVKVSCKASGYTFTSYWMHWVRQAPGQGLEWIGM
IHPNSGSTNYNEKFKSRVTMTVDTSTSTAYMELSSLRSEDTAVYYCARRY
YGSISPDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKENWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK.
1E7 humanized antibody heavy chain
1E7-huVH4-HC (IgG1):
(SEQ ID NO: 95)
QVQLVQSGAEVKKPGASVKVSCKASGYTFTSYWMHWVRQAPGQGLEWMGM
IHPNSGSTNYNEKFKSRVTMTVDTSTSTVYMELSSLRSEDTAVYYCARRY
YGSISPDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK.
1E7 humanized antibody heavy chain
1E7-huVH5-HC (IgG1):
(SEQ ID NO: 96)
QVQLVQSGAEVKKPGASVKVSCKASGYTFTSYWMHWVRQAPGQGLEWMGM
IHPNSGSTNYAQKFQGRVTMTRDTSTSTVYMELSSLRSEDTAVYYCARRY
YGSISPDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLFPPKPK
DTLMISRTPEVTCVVVDVSHEDPEVKENWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK.
1E7 humanized antibody heavy chain
1E7-huVH1-HC (IgG4):
(SEQ ID NO: 97)
QVQLVQSGAEVKKPGASVKLSCKASGYTFTSYWMHWVRQRPGQGLEWIGM
IHPNSGSTNYNEKFKSKATLTVDKSTSTAYMELSSLRSEDTAVYYCARRY
YGSISPDYWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK.
1E7 humanized antibody heavy chain
1E7-huVH2-HC (IgG4):
(SEQ ID NO: 98)
QVQLVQSGAEVKKPGASVKLSCKASGYTFTSYWMHWVRQAPGQGLEWIGM
IHPNSGSTNYNEKFKSRATLTVDTSTSTAYMELSSLRSEDTAVYYCARRY
YGSISPDYWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK.
1E7 humanized antibody heavy chain
1E7-huVH3-HC (IgG4):
(SEQ ID NO: 99)
QVQLVQSGAEVKKPGASVKVSCKASGYTFTSYWMHWVRQAPGQGLEWIGM
IHPNSGSTNYNEKFKSRVTMTVDTSTSTAYMELSSLRSEDTAVYYCARRY
YGSISPDYWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK.
1E7 humanized antibody heavy chain
1E7-huVH4-HC (IgG4):
(SEQ ID NO: 100)
QVQLVQSGAEVKKPGASVKVSCKASGYTFTSYWMHWVRQAPGQGLEWMGM
IHPNSGSTNYNEKFKSRVTMTVDTSTSTVYMELSSLRSEDTAVYYCARRY
YGSISPDYWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK.
1E7 humanized antibody heavy chain
1E7-huVH5-HC (IgG4):
(SEQ ID NO: 101)
QVQLVQSGAEVKKPGASVKVSCKASGYTFTSYWMHWVRQAPGQGLEWMGM
IHPNSGSTNYAQKFQGRVTMTRDTSTSTVYMELSSLRSEDTAVYYCARRY
YGSISPDYWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK.

According to some specific embodiments of the present invention, the antibody comprises: a light chain as shown in SEQ ID NO: 76, a heavy chain as shown in SEQ ID NO: 79, or a light chain as shown in SEQ ID NO: 77, a heavy chain as shown in SEQ ID NO: 79, or a light chain as shown in SEQ ID NO: 76, a heavy chain as shown in SEQ ID NO: 80, or a light chain as shown in SEQ ID NO: 77, a heavy chain as shown in SEQ ID NO: 80, or a light chain as shown in SEQ ID NO: 76, a heavy chain as shown in SEQ ID NO: 81, or a light chain as shown in SEQ ID NO: 77, a heavy chain as shown in SEQ ID NO: 81, or a light chain as shown in SEQ ID NO: 78, a heavy chain as shown in SEQ ID NO: 81, or a light chain as shown in SEQ ID NO: 77, a heavy chain as shown in SEQ ID NO: 82, or a light chain as shown in SEQ ID NO: 78, a heavy chain as shown in SEQ ID NO: 82, or a light chain as shown in SEQ ID NO: 77, a heavy chain as shown in SEQ ID NO: 83, or a light chain as shown in SEQ ID NO: 78, a heavy chain as shown in SEQ ID NO: 83, or a light chain as shown in SEQ ID NO: 76, a heavy chain as shown in SEQ ID NO: 84, or a light chain as shown in SEQ ID NO: 77, a heavy chain as shown in SEQ ID NO: 84, or a light chain as shown in SEQ ID NO: 76, a heavy chain as shown in SEQ ID NO: 85, or a light chain as shown in SEQ ID NO: 77, a heavy chain as shown in SEQ ID NO: 85, or a light chain as shown in SEQ ID NO: 76, a heavy chain as shown in SEQ ID NO: 86, or a light chain as shown in SEQ ID NO: 77, a heavy chain as shown in SEQ ID NO: 86, or a light chain as shown in SEQ ID NO: 78, a heavy chain as shown in SEQ ID NO: 86, or a light chain as shown in SEQ ID NO: 77, a heavy chain as shown in SEQ ID NO: 87, or a light chain as shown in SEQ ID NO: 78, a heavy chain as shown in SEQ ID NO: 87, or a light chain as shown in SEQ ID NO: 77, a heavy chain as shown in SEQ ID NO: 88, or a light chain as shown in SEQ ID NO: 78, a heavy chain as shown in SEQ ID NO: 88.

According to some specific embodiments of the present invention, the antibody comprises: a light chain as shown in SEQ ID NO: 89, a heavy chain as shown in SEQ ID NO: 92, or a light chain as shown in SEQ ID NO: 90, a heavy chain as shown in SEQ ID NO: 92, or a light chain as shown in SEQ ID NO: 89, a heavy chain as shown in SEQ ID NO: 93, or a light chain as shown in SEQ ID NO: 90, a heavy chain as shown in SEQ ID NO: 93, or a light chain as shown in SEQ ID NO: 89, a heavy chain as shown in SEQ ID NO: 94, or a light chain as shown in SEQ ID NO: 90, a heavy chain as shown in SEQ ID NO: 94, or a light chain as shown in SEQ ID NO: 91, a heavy chain as shown in SEQ ID NO: 94, or a light chain as shown in SEQ ID NO: 90, a heavy chain as shown in SEQ ID NO: 95, or a light chain as shown in SEQ ID NO: 91, a heavy chain as shown in SEQ ID NO: 95, or a light chain as shown in SEQ ID NO: 90, a heavy chain as shown in SEQ ID NO: 96, or a light chain as shown in SEQ ID NO: 91, a heavy chain as shown in SEQ ID NO: 96, or a light chain as shown in SEQ ID NO: 89, a heavy chain as shown in SEQ ID NO: 97, or a light chain as shown in SEQ ID NO: 90, a heavy chain as shown in SEQ ID NO: 97, or a light chain as shown in SEQ ID NO: 89, a heavy chain as shown in SEQ ID NO: 98, or a light chain as shown in SEQ ID NO: 90, a heavy chain as shown in SEQ ID NO: 98, or a light chain as shown in SEQ ID NO: 89, a heavy chain as shown in SEQ ID NO: 99, or a light chain as shown in SEQ ID NO: 90, a heavy chain as shown in SEQ ID NO: 99, or a light chain as shown in SEQ ID NO: 91, a heavy chain as shown in SEQ ID NO: 99, or a light chain as shown in SEQ ID NO: 90, a heavy chain as shown in SEQ ID NO: 100, or a light chain as shown in SEQ ID NO: 91, a heavy chain as shown in SEQ ID NO: 100, or a light chain as shown in SEQ ID NO: 90, a heavy chain as shown in SEQ ID NO: 101, or a light chain as shown in SEQ ID NO: 91, a heavy chain as shown in SEQ ID NO: 101.

According to some specific embodiments of the present invention, the antibody is a single chain antibody, a multimeric antibody, a CDR-grafted antibody or a small molecule antibody.

According to some specific embodiments of the present invention, the small molecule antibody includes at least one of a Fab antibody, a Fv antibody, a single domain antibody and a minimum recognition unit.

### Immune Cell, CAR-T Cell

The term “chimeric antigen receptor (CAR)” is a molecule that combines antibody-based specificity against a desired antigen (e.g., tumor antigen) with T cell receptor-activating intracellular domain to produce a chimeric protein that exhibits specific anti-tumor cell immune activity.

“CAR-T cell” refer to chimeric antigen receptor T cell, which can be understood as T cell that can express chimeric antigen receptor so that T cell can perform specific killing function.

In some embodiments, the present invention provides an immune cell, the immune cell carries the nucleic acid molecule described above, or expresses the antibody or antigen-binding fragment thereof described above. According to some specific embodiments of the present invention, the immune cell can express the aforementioned antibody or antigen-binding fragment thereof that specifically recognize Claudin18.2 in large quantities, so as to obtain the antibody or antigen-binding fragment thereof in large quantities in vitro. Furthermore, the immune cell can be reinfused in vivo to treat Claudin18.2-related diseases.

According to some specific embodiments of the present invention, the aforementioned immune cell may further include at least one of the following additional technical features:

According to some specific embodiments of the present invention, the immune cells include at least one of T lymphocytes, DC cells, NK cells, and NKT lymphocytes. The immune cells according to some embodiments of the present invention use the aforementioned antibody to recognize and kill target proteins, cells, tissues, organs, etc., and then perform biological functions.

In some embodiments, the present invention provides a CAR-T cell including a chimeric antigen receptor, wherein the chimeric antigen receptor includes: an extracellular region, the extracellular region includes a heavy chain variable region and a light chain variable region of a single-chain antibody, the light chain variable region has an amino acid sequence as shown in SEQ ID NO: 57-59 or SEQ ID NO: 65-67, and the heavy chain variable region has an amino acid sequence as shown in SEQ ID NO: 60-64 or SEQ ID NO: 68-72; a transmembrane region, the transmembrane region is connected to the extracellular region and embedded in the cell membrane of the T lymphocyte; and an intracellular region, the intracellular region is connected to the transmembrane region. CAR-T cells expressing this chimeric antigen receptor can kill tumor cells expressing Claudin18.2.

In some embodiments, the transmembrane region is an OX40 transmembrane region, an ICOS transmembrane region or a CD8 transmembrane region, preferably, the transmembrane region is a CD8 transmembrane region.

In some embodiments, the amino acid sequence of the CD8 trans-membrane region (TM) is shown in SEQ ID NO: 106:

| (SEQ ID NO: 106) | |
|---|---|
| | IYIWAPLAGTCGVLLLSLVITLYC |

In some embodiments, the amino acid sequence of the ICOS trans-membrane region (TM) is shown in SEQ ID NO: 107:

| (SEQ ID NO: 107) | |
|---|---|
| | FWLPIGCAAFVVVCILGCILICWL |

In some embodiments, the amino acid sequence of the OX40 trans-membrane region (TM) is shown in SEQ ID NO: 108:

| (SEQ ID NO: 108) | |
|---|---|
| | VAAILGLGLVLGLLGPLAILLALYLL |

In some embodiments, the intracellular segment is a 4-1BB intracellular segment and an ICOS or OX-40 intracellular segment; preferably, the intracellular segment is a 4-1BB intracellular segment and an ICOS intracellular segment.

The amino acid sequence of the intracellular segment of the 4-1BB immune co-stimulator is shown in SEQ ID NO: 109:

| (SEQ ID NO: 109) | |
|---|---|
| | KRGRKKLLYIFKQPFMRPVQTTQEEDGCSCRFPEEEEGGCEL |

The amino acid sequence of the intracellular segment of the ICOS immune co-stimulator is shown in SEQ ID NO: 110:

| (SEQ ID NO: 110) | |
|---|---|
| | CWLTKKKYSSSVHDPNGEYMFMRAVNTAKKSRLTDVTL |

In some embodiments, the extracellular region further comprises a CD8 hinge region. The amino acid sequence of the CD8 hinge region is shown in SEQ ID NO: 111:

(SEQ ID NO: 111)

TTTPAPRPPTPAPTIASQPLSLRPEACRPAAGGAVHTRGLDFACD

In some embodiments, the intracellular segment further comprises a CD3ζ chain. The amino acid sequence of the CD3ζ chain is shown in SEQ ID NO: 112:

(SEQ ID NO: 112)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR

In some embodiments, the N-terminus of the ICOS intracellular segment is connected to the C-terminus of the CD8 transmembrane region, the C-terminus of the ICOS intracellular segment is connected to the N-terminus of the 4-1BB intracellular segment, and the C-terminus of the 4-1BB intracellular segment is connected to the N-terminus of the CD3ζ chain.

According to an embodiment of the present invention, the structure of the chimeric antigen receptor is: signal peptide-Anti-Claudin 18.2 scfv-CD8 hinge+CD8TM-ICOS-4-1BB-CD3ζ. The Anti-Claudin18.2 scfv comprises a light chain variable region with an amino acid sequence as shown in SEQ ID NO: 57-59 or SEQ ID NO: 65-67, and the heavy chain variable region with an amino acid sequence as shown in SEQ ID NO: 60-64 or SEQ ID NO: 68-72.

### Nucleic Acid Molecule, Expression Vector, Recombinant Cell

In the process of preparing or obtaining these antibodies or chimeric antigen receptors, nucleic acid molecules expressing these antibodies or chimeric antigen receptors can be linked to different vectors and expressed in different cells to obtain corresponding antibodies or chimeric antigen receptors.

In some embodiments, the present invention provides a nucleic acid molecule, the nucleic acid molecule encodes the antibody or antigen-binding fragment thereof provided in the first aspect. The antibodies obtained from the nucleic acid molecules of some specific embodiments of the present invention can not only specifically target and bind to Claudin18.2 and have the same binding activity as the chimeric antibody, but also have a longer in vivo half-life.

According to some specific embodiments of the present invention, the aforementioned nucleic acid molecule may further include at least one of the following additional technical features:

According to some specific embodiments of the present invention, the nucleic acid molecule is DNA.

It should be noted that, for the nucleic acids mentioned in the specification and claims of the present invention, those skilled in the art should understand that they actually include any one or both of the complementary double chains. For convenience, in the present specification and claims, although only one chain is shown in most cases, another chain complementary thereto is actually also disclosed. In addition, the nucleic acid sequences in the present application include DNA form or RNA form, and disclosure of one of them means that the other is also disclosed.

In some embodiments, the present invention provides an expression vector, the expression vector carries the nucleic acid molecule described above. When the nucleic acid molecule described above is connected to the vector, the nucleic acid molecule can be directly or indirectly connected to the control elements on the vector, as long as these control elements can control the translation and expression of the nucleic acid molecule. Of course, these control elements can come directly from the carrier itself, or exogenous, that is, not from the carrier itself. Of course, the nucleic acid molecule can be operably linked to the control element. Here, “operably linked” refers to the connection of the exogenous gene to the vector, so that the control elements in the vector, such as transcription control sequence and translation control sequence, etc., can perform its expected function of regulating the transcription and translation of the exogenous gene. Of course, the nucleic acid molecule used to encode the heavy chain and light chain of an antibody can be inserted into different vectors independently, and it is common to insert into the same vector. Commonly used vectors can be, for example, plasmids, bacteriophages, and the like. After the expression vector according to some specific embodiments of the present invention is introduced into a suitable recipient cell, it can effectively realize the expression of the aforementioned antibody or antigen-binding fragment thereof that specifically recognizes Claudin18.2 under the mediation of the regulatory system, thereby realizing the mass acquisition of the antibody or antigen-binding fragment in vitro.

According to some specific embodiments of the present invention, the aforementioned expression vector may further include at least one of the following additional technical features:

According to some specific embodiments of the present invention, the expression vector is a eukaryotic expression vector or a virus. Then, the above-mentioned antibody or antigen-binding fragment thereof which specifically recognizes Claudin18.2 is expressed in suitable receptor cells, such as CHO cells.

According to some specific embodiments of the present invention, the virus is a lentivirus.

In some embodiments, the present invention provides a recombinant cell, the recombinant cell carries the nucleic acid molecule described above, or expresses the antibody or antigen-binding fragment thereof described above. The recombinant cells according to some specific embodiments of the present invention can be used for the in vitro expression and mass acquisition of the aforementioned antibodies or antigen-binding fragments specifically recognizing Claudin18.2.

According to some specific embodiments of the present invention, the aforementioned recombinant cell may further include at least one of the following additional technical features:

According to some specific embodiments of the present invention, the recombinant cell is obtained by introducing the aforementioned expression vector into a host cell.

According to some specific embodiments of the present invention, the expression vector is introduced into the host cell by electrotransduction.

According to some specific embodiments of the present invention, the recombinant cell is a eukaryotic cell.

According to some specific embodiments of the present invention, the recombinant cell is a mammalian cell.

According to some specific embodiments of the present invention, the eukaryotic cell does not include animal germ cell, oosperm or embryonic stem cell.

It should be noted that the recombinant cell of the present invention is not particularly limited and may be prokaryotic cell, eukaryotic cell or bacteriophage. The prokaryotic cell may be Escherichia coli, Bacillus subtilis, Streptomyces or Proteus mirabilis. The eukaryotic cell may be fungi such as Pichia pastori, Saccharomyces cerevisiae, Schizosaccharomyces pombe, Trichoderma, insect cell such as S. frugiperda, plant cell such as tobacco, mammalian cell such as BHK cell, CHO cell, COS cell, myeloma cell, etc. In some embodiments, the recombinant cell of the present invention is preferably mammalian cell, including BHK cell, CHO cell, NSO cell or COS cell, and do not include animal germ cell, oosperm or embryonic stem cell.

It should be noted that the “suitable conditions” described in the specification of this application refer to conditions suitable for the expression of the recombinant antibodies described in this application. It is easily understood by those skilled in the art that conditions suitable for expression of recombinant antibodies include, but are not limited to, suitable transformation or transfection methods, suitable transformation or transfection conditions, healthy host cell status, suitable host cell density, suitable cell culture environment, and suitable cell culture time. The “suitable conditions” are not particularly limited, and those skilled in the art can optimize the most suitable conditions for expressing the recombinant antibody according to the specific environment of the laboratory.

### Pharmaceutical Composition, Kit and Pharmaceutical Use and Use in the Preparation of Kit

In some embodiments, the present invention provides a pharmaceutical composition, the pharmaceutical composition includes the aforementioned antibody, nucleic acid molecule, expression vector, immune cell, recombinant cell or CAR-T cell. As mentioned above, the antibody, as well as the nucleic acid molecule, expression vector, immune cell, recombinant cell or CAR-T cell that can obtain the antibody after expression can not only specifically target and bind to Claudin18.2, and have the same binding activity as the chimeric antibody, but also have a longer in vivo half-life. Therefore, the antibody or expressed antibody contained in the pharmaceutical composition according to some specific embodiments of the present invention can specifically target and bind to Claudin18.2, has strong specificity, and exerts a good targeting effect, thereby realizing the biological effects of other drugs in the pharmaceutical composition, such as the activity inhibition of Claudin18.2 molecule, the killing of cells expressing Claudin18.2 molecule, and the like.

In some embodiments, these pharmaceutical compositions further include a pharmaceutically acceptable carrier, including any solvents, solid excipients, diluents, binders, disintegrants, or other liquid excipients, dispersing agents, flavoring or suspending agents, surfactants, isotonic agents, thickeners, emulsifiers, preservatives, solid binders, glidants or lubricants, etc., which are suitable for specific target dosage forms. Except insofar as any conventional excipients incompatible with the compounds disclosed herein, such as by producing any undesirable biological effect or otherwise interacting in a deleterious manner with any other components of the pharmaceutically acceptable composition, its use is contemplated to be within the scope of this invention.

For example, the antibodies of the present invention can be incorporated into pharmaceutical compositions suitable for parenteral administration (e.g., intravenous, subcutaneous, intraperitoneal, intramuscular). These pharmaceutical compositions can be prepared in various forms. For example, liquid, semi-solid and solid dosage forms, including but not limited to liquid solutions (for example, injection solutions and infusion solutions), dispersions or suspensions, tablets, pills, powders, liposomes, and suppositories. Typical pharmaceutical compositions are in the form of injection solutions or infusion solutions. The antibody can be administered by intravenous infusion or injection, or intramuscular or subcutaneous injection.

It should be noted that the composition includes combinations separated in time and/or space, as long as they can work together to achieve the purpose of the present invention. For example, the components contained in the composition may be administered to the subject as a whole, or may be administered to the subject separately. When the components contained in the composition are administered to the subject separately, the respective components may be administered to the subject simultaneously or sequentially.

In some embodiments, the present invention provides the use of the aforementioned antibody, the aforementioned nucleic acid molecule, the aforementioned expression vector, immune cell, recombinant cell, CAR-T cell or the aforementioned pharmaceutical composition in the manufacture of a medicament for the treatment or prevention of Claudin18.2 related diseases. As mentioned above, the antibody, as well as the nucleic acid molecule, expression vector, immune cell, recombinant cell or CAR-T cell that can obtain the antibody after expression can not only specifically target and bind to Claudin18.2, and have the same binding activity as the chimeric antibody, but also have a longer in vivo half-life. Therefore, drugs containing the above substances can also effectively target and bind to Claudin18.2, have a longer half-life in vivo, and can effectively kill cells expressing Claudin18.2 molecules.

According to some specific embodiments of the present invention, the aforementioned use may further include at least one of the following additional technical features:

According to some specific embodiments of the present invention, the Claudin18.2 related diseases include: at least one of gastric cancer, esophageal cancer, pancreatic cancer, lung cancer, colon cancer and rectal cancer. Those skilled in the art will appreciate that the drug of the present invention can be effective for any tissue or organ that highly expresses Claudin18.2, and therefore, the drug can be used to treat or alleviate lesions of the corresponding tissue or organ.

In some embodiments, the present invention provides a kit for detecting Claudin18.2, the kit includes any one of the antibody described above. The kit provided by the present invention can be used, for example, for immunoblotting, immunoprecipitation, etc., which involve the use of the specific binding properties of Claudin18.2 antigen and antibody to detect. These kits may contain any one or more of the following: antagonist, Claudin18.2 antibody or drug reference material; protein purification column; immunoglobulin affinity purification buffer; cell assay diluent; instructions or literature, etc. The Claudin18.2 antibodies can be used in different types of diagnostic tests, for example, it can detect the presence of various diseases or drugs, toxins or other proteins in vitro or in vivo. For example, the subject's serum or blood can be tested for related diseases. Such as cancers or tumors, these cancers or tumors can be any unregulated cell growth.

In some embodiments, the present invention provides the use of the aforementioned antibody, the aforementioned nucleic acid molecule, the aforementioned expression vector or the aforementioned recombinant cell in the manufacture of a kit for detecting Claudin 18.2 or diagnosing Claudin 18.2 related diseases. The Claudin18.2 antibodies can be used in combination with any detection reagent or therapeutic agent, for example, in combination with diagnostic nuclides, nanomaterials, etc., to detect the target site through the radioactivity of the nuclide, so as to obtain information about the target site; it can also be used in combination with therapeutic nuclides to use the radioactivity of the nuclides to specifically kill target cells, tissues, etc.

The scheme of the present invention will be explained below with reference to embodiments. Those skilled in the art will appreciate that the following embodiments are only used to illustrate the present invention and should not be construed as limiting the scope of the present invention. If the specific technology or conditions are not indicated in the examples, the technology or conditions described in the literature in the art or the product descriptions are performed. If the reagents or instruments used are not specified by the manufacturers, they are all conventional products that are commercially available.

### Example 1 Design and Analysis of Humanized Antibody Sequences

The applicant used Discovery Studio and Schrödinger Antibody Modeling for the 1B6 mouse antibody sequence (SEQ ID NO: 102, 103) and the 1E7 mouse antibody sequence (SEQ ID NO: 104, 105), respectively, and used PDB BLAST to retrieve the 10 antibody crystal structure models with the closest sequences (with a structural resolution higher than 2.5 angstroms), compared them with the automatic modeling models, and selected the optimal structural model. IgBLAST was used to align the 1B6 and 1E7 murine antibody sequences with the human GermLine sequences, and the GermLine with the highest homology was selected. Based on the modeled structural model, human GermLine sequence alignment results and the characteristics of different subtypes (IgG1, IgG4), the murine FR region was replaced with the human FR region, and some key residues in the human FR region were back mutated to murine residues. The humanization design was completed when the degree of humanization of the variable region (excluding the CDR region) was greater than 90%. The specific sequence of the obtained human antibody 1B6 is shown in SEQ ID NO:57-64, and the specific sequence of the obtained human antibody 1E7 is shown in SEQ ID NO:65-72. In addition, the inventors replaced the murine light chain constant region with the human light chain constant region (Kappa) (SEQ ID NO: 73), and replaced the murine heavy chain constant region with the heavy chain constant region of the human IgG1 antibody (SEQ ID NO: 74) or the heavy chain constant region of the human IgG4 antibody (SEQ ID NO: 75).

Murine antibody 1B6 light chain variable region (1B6-VL):
(SEQ ID NO: 102)
DIVMTQSPSSLTVTAGEKVTMSCKSSQSLFNSGNQKNYLTWYQQKPGQPPKLLIYW
ASTRESGVPDRFTGSGSGTDFTLTISSVQAEDLAVYFCQNDYSYPLTFGAGTKLELK.
Murine antibody 1B6 heavy chain variable region (1B6-VH):
(SEQ ID NO: 103)
EVQLQQSGPELVRPGASVKMSCKASGYTFTDYNMHWVKQSHGKSLEWIGYINPNN
GGTSYNQKFKGKATLTVNKSSSTAYMELRSLTSEDSAVYYCVTTRYLAVWGTGTTVTVS
S.
Murine antibody 1E7 light chain variable region (1E7-VL):
(SEQ ID NO: 104)
DIVMTQSPSSLSVTAGEKVTMSCKSSQSLLNSGNQKNYLTWYQQKPGQPPKLLIYW
ASTRESGVPDRFTGSGSGTDFTLTISSVQAEDLAVYYCONDYSYPFTFGSGTKLEIK.
Murine antibody 1E7 heavy chain variable region (1E7-VH):
(SEQ ID NO: 105)
QVQLQQPGSELVKPGASVKLSCKASGYTFTSYWMHWVKQRPGQGLEWIGMIHPNS
GSTNYNEKFKSKATLTVDKSSSTAYMQLSSLTSEDSAVYYCARRYYGSISPDYWGQGTTL
TVSS.
Humanized 1B6 antibody light chain variable region 1 (1B6-huVL1):
(SEQ ID NO: 57)
DIVMTQSPSSLSVSVGDRVTMTCKSSQSLFNSGNQKNYLTWYQQKPGKAPKLLIYW
ASTRESGVPDRFSGSGSGTDFTLTISSVQPEDFATYFCQNDYSYPLTFGGGTKLEIK.
Humanized 1B6 antibody light chain variable region 2 (1B6-huVL2):
(SEQ ID NO: 58)
DIQMTQSPSSLSASVGDRVTMTCKSSQSLFNSGNQKNYLTWYQQKPGKAPKLLIYW
ASTRESGVPSRFSGSGSGTDFTLTISSLQPEDFATYYCONDYSYPLTFGGGTKLEIK.
Humanized 1B6 antibody light chain variable region 3 (1B6-huVL3):
(SEQ ID NO: 59)
DIQMTQSPSSLSASVGDRVTITCRSSQSLFNSGNQKNYLTWYQQKPGKAPKLLIYWA
STRESGVPSRFSGSGSGTDFTLTISSLQPEDFATYYCONDYSYPLTFGGGTKLEIK.
Humanized 1B6 antibody heavy chain variable region 1 (1B6-huVH1):
(SEQ ID NO: 60)
EVQLVQSGAEVKKPGASVKMSCKASGYTFTDYNMHWVRQAPGKSLEWIGYINPNN
GGTSYNQKFKGKATLTVNKSSSTAYMELSSLRSEDTAVYYCVTTRYLAVWGQGTTVTVS
S.
Humanized 1B6 antibody heavy chain variable region 2 (1B6-huVH2):
(SEQ ID NO: 61)
QVQLVQSGAEVKKPGASVKMSCKASGYTFTDYNMHWVRQAPGQRLEWIGYINPN
NGGTSYNQKFKGKATLTVDTSASTAYMELSSLRSEDTAVYYCVTTRYLAVWGQGTTVTV
SS.
Humanized 1B6 antibody heavy chain variable region 3 (1B6-huVH3):
(SEQ ID NO: 62)
QVQLVQSGAEVKKPGASVKVSCKASGYTFTDYNMHWVRQAPGQRLEWIGYINPN
NGGTSYNQKFKGRVTITVDTSASTAYMELSSLRSEDTAVYYCVTTRYLAVWGQGTTVTV
SS.
Humanized 1B6 antibody heavy chain variable region 4 (1B6-huVH4):
(SEQ ID NO: 63)
QVQLVQSGAEVKKPGASVKVSCKASGYTFTDYNMHWVRQAPGQRLEWMGYINPN
NGGTSYNQKFQGRVTITVDTSASTAYMELSSLRSEDTAVYYCVTTRYLAVWGQGTTVTV
SS.
Humanized 1B6 antibody heavy chain variable region 5 (1B6-huVH5):
(SEQ ID NO: 64)
QVQLVQSGAEVKKPGASVKVSCKASGYTFTDYNMHWVRQAPGQRLEWMGYINPN
NGGTSYSQKFQGRVTITRDTSASTAYMELSSLRSEDTAVYYCVTTRYLAVWGQGTTVTVS
S.
Humanized 1E7 antibody light chain variable region 1 (1E7-huVL1):
(SEQ ID NO: 65)
DIVMTQSPSSLSVSVGDRVTMTCKSSQSLLNSGNQKNYLTWYQQKPGQPPKLLIYW
ASTRESGVPDRFSGSGSGTDFTLTISSVQPEDFATYYCONDYSYPFTFGGGTKLEIK.
Humanized 1E7 antibody light chain variable region 2 (1E7-huVL2):
(SEQ ID NO: 66)
DIQMTQSPSSLSASVGDRVTMTCKSSQSLLNSGNQKNYLTWYQQKPGKAPKLLIYW
ASTRESGVPSRFSGSGSGTDFTLTISSLQPEDFATYYCONDYSYPFTFGGGTKLEIK.
Humanized 1E7 antibody light chain variable region 3 (1E7-huVL3):
(SEQ ID NO: 67)
DIQMTQSPSSLSASVGDRVTITCRSSQSLLNSGNQKNYLTWYQQKPGKAPKLLIYWA
STRESGVPSRFSGSGSGTDFTLTISSLQPEDFATYYCONDYSYPFTFGGGTKLEIK.
Humanized 1E7 antibody heavy chain variable region 1 (1E7-huVH1):
(SEQ ID NO: 68)
QVQLVQSGAEVKKPGASVKLSCKASGYTFTSYWMHWVRQRPGQGLEWIGMIHPNS
GSTNYNEKFKSKATLTVDKSTSTAYMELSSLRSEDTAVYYCARRYYGSISPDYWGQGTTV
TVSS.
Humanized 1E7 antibody heavy chain variable region 2 (1E7-huVH2):
(SEQ ID NO: 69)
QVQLVQSGAEVKKPGASVKLSCKASGYTFTSYWMHWVRQAPGQGLEWIGMIHPN
SGSTNYNEKFKSRATLTVDTSTSTAYMELSSLRSEDTAVYYCARRYYGSISPDYWGQGTT
VTVSS.
Humanized 1E7 antibody heavy chain variable region 3 (1E7-huVH3):
(SEQ ID NO: 70)
QVQLVQSGAEVKKPGASVKVSCKASGYTFTSYWMHWVRQAPGQGLEWIGMIHPN
SGSTNYNEKFKSRVTMTVDTSTSTAYMELSSLRSEDTAVYYCARRYYGSISPDYWGQGTT
VTVSS.
Humanized 1E7 antibody heavy chain variable region 4 (1E7-huVH4):
(SEQ ID NO: 71)
QVQLVQSGAEVKKPGASVKVSCKASGYTFTSYWMHWVRQAPGQGLEWMGMIHP
NSGSTNYNEKFKSRVTMTVDTSTSTVYMELSSLRSEDTAVYYCARRYYGSISPDYWGQG
TTVTVSS.
Humanized 1E7 antibody heavy chain variable region 5 (1E7-huVH5):
(SEQ ID NO: 72)
QVQLVQSGAEVKKPGASVKVSCKASGYTFTSYWMHWVRQAPGQGLEWMGMIHP
NSGSTNYAQKFQGRVTMTRDTSTSTVYMELSSLRSEDTAVYYCARRYYGSISPDYWGQG
TTVTVSS.
Human light chain constant region LC (Kappa):
(SEQ ID NO: 73)
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC.
Human IgG1 heavy chain constant region HC:
(SEQ ID NO: 74)
ASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKS
RWQQGNVFSCSVMHEALHNHYTQKSLSLSPGK.
Human IgG4 heavy chain constant region HC:
(SEQ ID NO: 75)
ASTKGPSVFPLAPCSRSTSESTAALGCLVKDYFPEPVTVSWNSGALTSGVHTFPAVLQ
SSGLYSLSSVVTVPSSSLGTKTYTCNVDHKPSNTKVDKRVESKYGPPCPPCPAPEFLGGPS
VFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLYSRLTVDKSRW
QEGNVFSCSVMHEALHNHYTQKSLSLSLGK.

### Example 2 Preparation and Identification of Humanized Antibodies

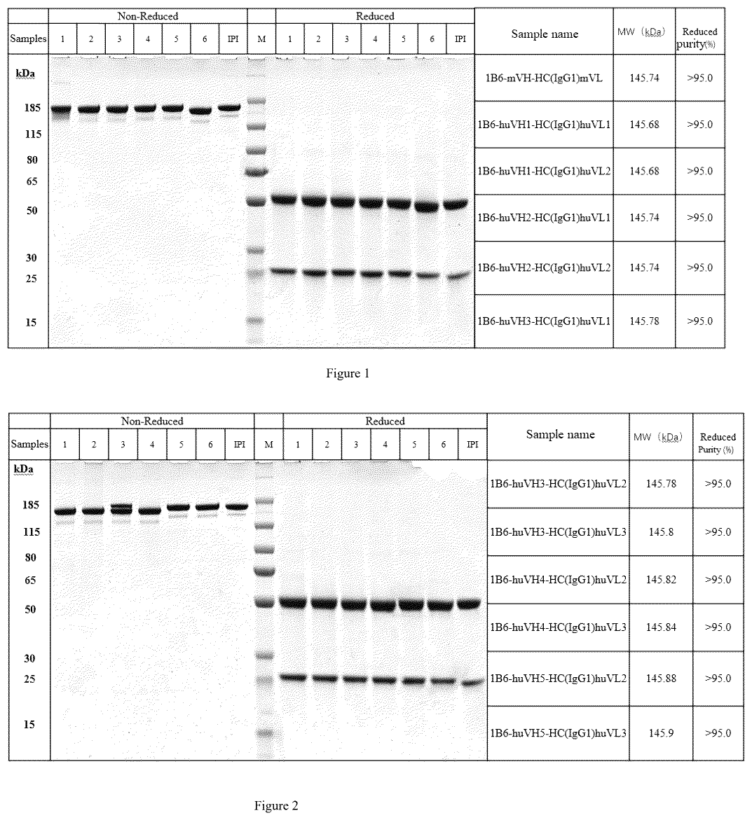

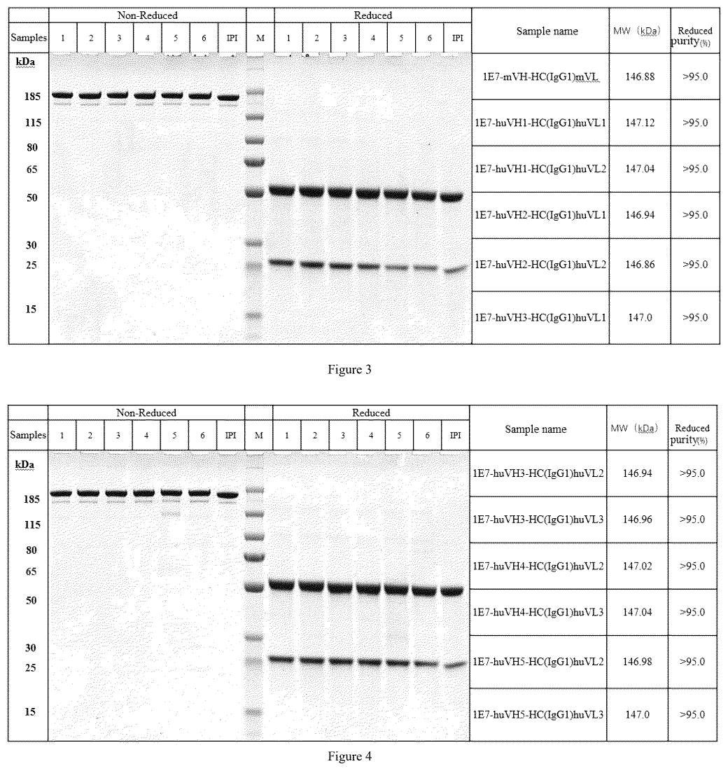

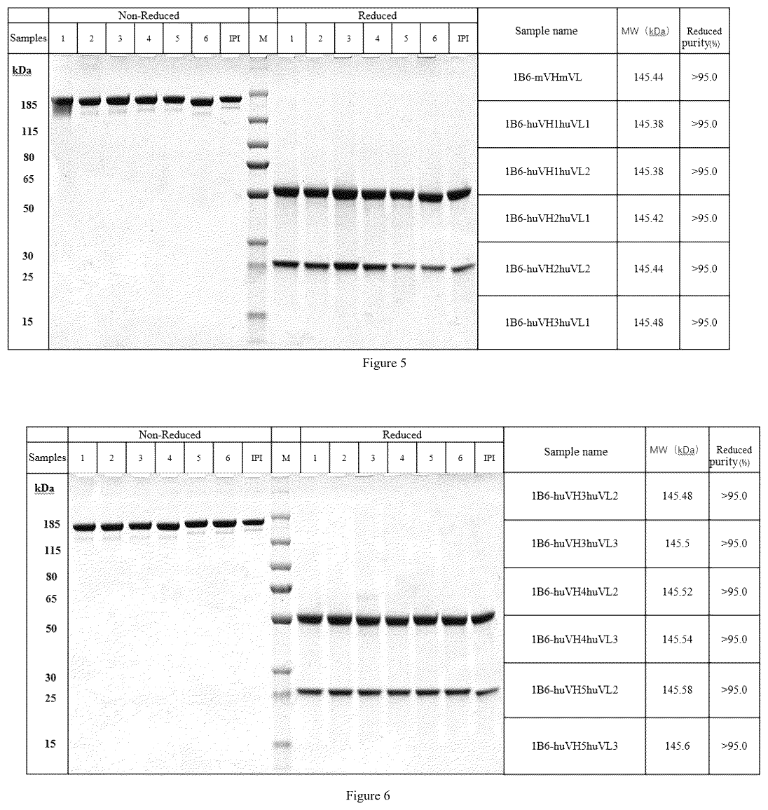

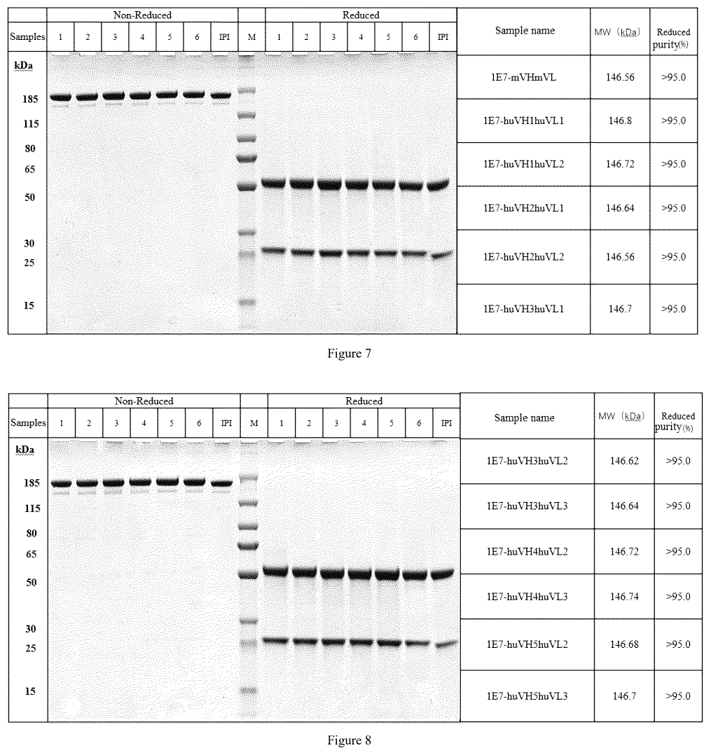

According to the results of the humanized design in Example 1, the sequence was constructed into two subtypes of pcDNA3.4 vectors, and plasmids were obtained through PCR, restriction digestion, ligation, transformation, identification, sequencing, alignment, and extraction. According to the expression combination recommended after humanization design in Example 1 (Table 1), the above plasmid was expressed in CHO cells for 7 days as required using the ExpiCHO-S expression system, and the expression level was measured by the Gator label-free bioanalyzer on the 6th day of expression. On the 7th day, the CHO cell expression fluid was collected, centrifuged, filtered with a filter, and the supernatant was taken. The recombinant antibody was purified using Protein-A affinity chromatography. The protein samples obtained by the Protein A column were identified by SDS-PAGE. The specific experimental results are shown in FIGS. 1-8. The purity of the reduced band was calculated by ImageJ software according to the peak area normalization method. Compared with the reference product IPI, the non-reduced band molecular weight is about 150 kDa, and the purity is greater than 90%; the reduced heavy chain molecular weight is about 55 kDa, the light chain molecular weight is about 25 kDa, and the purity of the heavy chain plus the light chain is greater than 90%. All humanized designed antibodies meet the characteristics of antibody molecular weight and have good purity, meeting the druggability requirements.

TABLE 1

| | The heavy | The light |
|---|---|---|
| | chain amino | chain amino |
| Sample name | acid sequence | acid sequence |
| 1B6-huVH1-HC (IgG1) huVL1 | SEQ ID NO: 79 | SEQ ID NO: 76 |
| 1B6-huVH1-HC (IgG1) huVL2 | SEQ ID NO: 79 | SEQ ID NO: 77 |
| 1B6-huVH2-HC (IgG1) huVL1 | SEQ ID NO: 80 | SEQ ID NO: 76 |
| 1B6-huVH2-HC (IgG1) huVL2 | SEQ ID NO: 80 | SEQ ID NO: 77 |
| 1B6-huVH3-HC (IgG1) huVL1 | SEQ ID NO: 81 | SEQ ID NO: 76 |
| 1B6-huVH3-HC (IgG1) huVL2 | SEQ ID NO: 81 | SEQ ID NO: 77 |
| 1B6-huVH3-HC (IgG1) huVL3 | SEQ ID NO: 81 | SEQ ID NO: 78 |
| 1B6-huVH4-HC (IgG1) huVL2 | SEQ ID NO: 82 | SEQ ID NO: 77 |
| 1B6-huVH4-HC (IgG1) huVL3 | SEQ ID NO: 82 | SEQ ID NO: 78 |
| 1B6-huVH5-HC (IgG1) huVL2 | SEQ ID NO: 83 | SEQ ID NO: 77 |
| 1B6-huVH5-HC (IgG1) huVL3 | SEQ ID NO: 83 | SEQ ID NO: 78 |
| 1E7-huVH1-HC (IgG1) huVL1 | SEQ ID NO: 92 | SEQ ID NO: 89 |
| 1E7-huVH1-HC (IgG1) huVL2 | SEQ ID NO: 92 | SEQ ID NO: 90 |
| 1E7-huVH2-HC (IgG1) huVL1 | SEQ ID NO: 93 | SEQ ID NO: 89 |
| 1E7-huVH2-HC (IgG1) huVL2 | SEQ ID NO: 93 | SEQ ID NO: 90 |
| 1E7-huVH3-HC (IgG1) huVL1 | SEQ ID NO: 94 | SEQ ID NO: 89 |
| 1E7-huVH3-HC (IgG1) huVL2 | SEQ ID NO: 94 | SEQ ID NO: 90 |
| 1E7-huVH3-HC (IgG1) huVL3 | SEQ ID NO: 94 | SEQ ID NO: 91 |
| 1E7-huVH4-HC (IgG1) huVL2 | SEQ ID NO: 95 | SEQ ID NO: 90 |
| 1E7-huVH4-HC (IgG1) huVL3 | SEQ ID NO: 95 | SEQ ID NO: 91 |
| 1E7-huVH5-HC (IgG1) huVL2 | SEQ ID NO: 96 | SEQ ID NO: 90 |
| 1E7-huVH5-HC (IgG1) huVL3 | SEQ ID NO: 96 | SEQ ID NO: 91 |
| 1B6-huVH1huVL1 | SEQ ID NO: 84 | SEQ ID NO: 76 |
| 1B6-huVH1huVL2 | SEQ ID NO: 84 | SEQ ID NO: 77 |
| 1B6-huVH2huVL1 | SEQ ID NO: 85 | SEQ ID NO: 76 |
| 1B6-huVH2huVL2 | SEQ ID NO: 85 | SEQ ID NO: 77 |
| 1B6-huVH3huVL1 | SEQ ID NO: 86 | SEQ ID NO: 76 |
| 1B6-huVH3huVL2 | SEQ ID NO: 86 | SEQ ID NO: 77 |
| 1B6-huVH3huVL3 | SEQ ID NO: 86 | SEQ ID NO: 78 |
| 1B6-huVH4huVL2 | SEQ ID NO: 87 | SEQ ID NO: 77 |
| 1B6-huVH4huVL3 | SEQ ID NO: 87 | SEQ ID NO: 78 |
| 1B6-huVH5huVL2 | SEQ ID NO: 88 | SEQ ID NO: 77 |
| 1B6-huVH5huVL3 | SEQ ID NO: 88 | SEQ ID NO: 78 |
| 1E7-huVH1huVL1 | SEQ ID NO: 97 | SEQ ID NO: 89 |
| 1E7-huVH1huVL2 | SEQ ID NO: 97 | SEQ ID NO: 90 |
| 1E7-huVH2huVL1 | SEQ ID NO: 98 | SEQ ID NO: 89 |
| 1E7-huVH2huVL2 | SEQ ID NO: 98 | SEQ ID NO: 90 |
| 1E7-huVH3huVL1 | SEQ ID NO: 99 | SEQ ID NO: 89 |
| 1E7-huVH3huVL2 | SEQ ID NO: 99 | SEQ ID NO: 90 |
| 1E7-huVH3huVL3 | SEQ ID NO: 99 | SEQ ID NO: 91 |
| 1E7-huVH4huVL2 | SEQ ID NO: 100 | SEQ ID NO: 90 |
| 1E7-huVH4huVL3 | SEQ ID NO: 100 | SEQ ID NO: 91 |
| 1E7-huVH5huVL2 | SEQ ID NO: 101 | SEQ ID NO: 90 |
| 1E7-huVH5huVL3 | SEQ ID NO: 101 | SEQ ID NO: 91 |

### Example 3 Assessment of Aggregate Content of Humanized Antibodies (SEC)

The inventors collected data using an Agilent HPLC 1100 instrument, an XBridge BEH200 Å, SEC 3.5 μm, 7.8×300 mm (batch number: ZL20200823) chromatographic column, with a mobile phase of 0.15M PB (pH 7.4), 20 μL of sample was loaded at a flow rate of 0.8 mL/min, and the detector (parameters: detection wavelength 280 nm, bandwidth 16 nm, reference wavelength 360 nm, bandwidth 100 nm, peak width (response time)>0.1 min (2 s); slit 4 nm; negative absorbance baseline 100 mAU). The specific data are shown in Table 2. Except for antibody 1E7-huVH5huVL3 (with a monomer ratio of 67.67%), the monomer ratios of the remaining antibodies are all greater than 90%. The monomer ratios of most humanized antibodies are better or equivalent to those of chimeric antibodies, indicating that the humanized designed antibodies have good uniformity and meet the druggability requirements.

TABLE 2

| | | | | Low |
|---|---|---|---|---|
| | Monomer | High | | molecular |
| | retention | poly- | Mono- | weight |
| | time | mer | mer | substance |
| Sample name | (min) | (%) | (%) | (%) |
| 1B6-mVH-HC (IgG1) mVL | 8.64 | 1.94 | 98.06 | / |
| 1B6-huVH1-HC (IgG1) huVL1 | 8.61 | 1.96 | 97.34 | 0.70 |
| 1B6-huVH1-HC (IgG1) huVL2 | 8.59 | 1.06 | 98.94 | / |
| 1B6-huVH2-HC (IgG1) huVL1 | 8.66 | 1.01 | 98.56 | 0.43 |
| 1B6-huVH2-HC (IgG1) huVL2 | 8.64 | 1.13 | 98.87 | / |
| 1B6-huVH3-HC (IgG1) huVL1 | 8.66 | 1.42 | 98.17 | 0.41 |
| 1B6-huVH3-HC (IgG1) huVL2 | 8.64 | 1.41 | 98.08 | 0.50 |
| 1B6-huVH3-HC (IgG1) huVL3 | 8.64 | 1.62 | 98.38 | / |
| 1B6-huVH4-HC (IgG1) huVL2 | 8.68 | 1.65 | 98.35 | / |
| 1B6-huVH4-HC (IgG1) huVL3 | 8.69 | 1.29 | 98.71 | / |
| 1B6-huVH5-HC (IgG1) huVL2 | 8.68 | 1.54 | 97.89 | 0.57 |
| 1B6-huVH5-HC (IgG1) huVL3 | 8.69 | 1.13 | 98.46 | 0.41 |
| 1E7-mVH-HC (IgG1) mVL | 8.69 | 1.35 | 98.65 | / |
| 1E7-huVH1-HC (IgG1) huVL1 | 8.68 | 0.39 | 99.14 | 0.47 |
| 1E7-huVH1-HC (IgG1) huVL2 | 8.66 | 0.47 | 99.53 | / |
| 1E7-huVH2-HC (IgG1) huVL1 | 8.72 | 0.32 | 99.68 | / |
| 1E7-huVH2-HC (IgG1) huVL2 | 8.70 | 1.16 | 98.55 | 0.30 |
| 1E7-huVH3-HC (IgG1) huVL1 | 8.73 | 0.40 | 99.26 | 0.34 |
| 1E7-huVH3-HC (IgG1) huVL2 | 8.71 | 0.37 | 99.43 | 0.20 |
| 1E7-huVH3-HC (IgG1) huVL3 | 8.72 | 0.58 | 99.42 | / |
| 1E7-huVH4-HC (IgG1) huVL2 | 8.73 | 0.39 | 99.21 | 0.40 |
| 1E7-huVH4-HC (IgG1) huVL3 | 8.74 | 0.69 | 98.79 | 0.52 |
| 1E7-huVH5-HC (IgG1) huVL2 | 8.77 | 4.16 | 90.37 | 5.46 |
| 1E7-huVH5-HC (IgG1) huVL3 | 8.77 | 0.52 | 98.44 | 1.04 |
| 1B6-mVHmVL | 8.74 | 1.22 | 98.78 | / |
| 1B6-huVH1huVL1 | 8.69 | 0.97 | 99.03 | / |
| 1B6-huVH1huVL2 | 8.68 | 0.98 | 99.02 | / |
| 1B6-huVH2huVL1 | 8.74 | 1.00 | 99.00 | / |
| 1B6-huVH2huVL2 | 8.73 | 0.66 | 99.34 | / |
| 1B6-huVH3huVL1 | 8.74 | 1.07 | 98.93 | / |
| 1B6-huVH3huVL2 | 8.73 | 0.41 | 99.59 | / |
| 1B6-huVH3huVL3 | 8.74 | 1.60 | 98.40 | / |
| 1B6-huVH4huVL2 | 8.77 | 1.70 | 98.30 | / |
| 1B6-huVH4huVL3 | 8.78 | 1.94 | 98.06 | / |
| 1B6-huVH5huVL2 | 8.77 | 0.90 | 99.10 | / |
| 1B6-huVH5huVL3 | 8.78 | 4.18 | 95.82 | / |
| 1E7-mVHmVL | 8.79 | 1.43 | 98.57 | / |
| 1E7-huVH1huVL1 | 8.75 | 1.53 | 98.47 | / |
| 1E7-huVH1huVL2 | 8.73 | 1.07 | 98.93 | / |
| 1E7-huVH2huVL1 | 8.79 | 0.78 | 99.22 | / |
| 1E7-huVH2huVL2 | 8.77 | 1.19 | 98.81 | / |
| 1E7-huVH3huVL1 | 8.80 | 5.34 | 94.66 | / |
| 1E7-huVH3huVL2 | 8.78 | 1.89 | 98.11 | / |
| 1E7-huVH3huVL3 | 8.79 | 2.33 | 97.67 | / |
| 1E7-huVH4huVL2 | 8.79 | 1.04 | 98.96 | / |
| 1E7-huVH4huVL3 | 8.80 | 1.05 | 98.95 | / |
| 1E7-huVH5huVL2 | 8.83 | 3.72 | 96.28 | / |
| 1E7-huVH5huVL3 | 8.84 | 32.33 | 67.67 | / |

### Example 4 Thermal Stability Evaluation of Humanized Antibodies (DSF)

In an eight-well tube strip or a 96-well plate, samples diluted to 0.2 mg/mL with 1×PBS (pH7.4) were added, and then 100×SYPRO Orange working solution was added to make the final concentration 5×. The final volume was 20 μL. The tube wall was flicked to mix, and centrifuged at 2000 rpm for 10 seconds; 3 replicates were prepared for each sample. The samples were placed on an ABI7500 Fast Real-Time PCR instrument, the melting curve was selected as the experimental type, the continuous mode was adopted, the scanning temperature was 25-99° C., the equilibrium was 25° C. for 5 min, the heating rate was 1%, the reporter group was ROX, and the quencher group was None. Result determination: the temperature corresponding to the first peak and valley of the derivative function of the melting curve was determined as the denaturation temperature Tm1 of the protein, and the temperature corresponding to the second peak and valley was determined as the denaturation temperature Tm2 of the protein. The specific experimental results are shown in Table 3. The minimum Tm values of all antibodies are greater than 64° C., indicating that the humanized antibodies have good thermal stability and meet the druggability requirements.

TABLE 3

| Sample name | Tm 1 (° C.) | Tm 2 (° C.) |
|---|---|---|
| 1B6-mVH-HC (IgG1) mVL | 77.2 | N/A |
| 1B6-huVH1-HC (IgG1) huVL1 | 68.95 | 77.04 |
| 1B6-huVH1-HC (IgG1) huVL2 | 69.19 | 77.26 |
| 1B6-huVH2-HC (IgG1) huVL1 | 68.76 | 77.94 |
| 1B6-huVH2-HC (IgG1) huVL2 | 68.92 | 77.79 |
| 1B6-huVH3-HC (IgG1) huVL1 | 69.23 | 79.8 |
| 1B6-huVH3-HC (IgG1) huVL2 | 69.28 | 78.88 |
| 1B6-huVH3-HC (IgG1) huVL3 | 69.08 | 77.76 |
| 1B6-huVH4-HC (IgG1) huVL2 | 68.77 | 78.39 |
| 1B6-huVH4-HC (IgG1) huVL3 | 69.27 | 77.61 |
| 1B6-huVH5-HC (IgG1) huVL2 | 74.41 | N/A |
| 1B6-huVH5-HC (IgG1) huVL3 | 73.91 | N/A |
| 1E7-mVH-HC (IgG1) mVL | 75.35 | N/A |
| 1E7-huVH1-HC (IgG1) huVL1 | 69.05 | 75.60 |
| 1E7-huVH1-HC (IgG1) huVL2 | 73.75 | N/A |
| 1E7-huVH2-HC (IgG1) huVL1 | 69.05 | 76.01 |
| 1E7-huVH2-HC (IgG1) huVL2 | 72.92 | N/A |
| 1E7-huVH3-HC (IgG1) huVL1 | 69.08 | 76.08 |
| 1E7-huVH3-HC (IgG1) huVL2 | 74.22 | N/A |
| 1E7-huVH3-HC (IgG1) huVL3 | 73.29 | N/A |
| 1E7-huVH4-HC (IgG1) huVL2 | 73.85 | N/A |
| 1E7-huVH4-HC (IgG1) huVL3 | 72.95 | N/A |
| 1E7-huVH5-HC (IgG1) huVL2 | 71.22 | N/A |
| 1E7-huVH5-HC (IgG1) huVL3 | 70.38 | N/A |
| 1B6-mVHmVL | 65.16 | 75.31 |
| 1B6-huVH1huVL1 | 65.43 | 75.50 |
| 1B6-huVH1huVL2 | 65.40 | 75.65 |
| 1B6-huVH2huVL1 | 65.37 | 76.09 |
| 1B6-huVH2huVL2 | 65.16 | 76.18 |
| 1B6-huVH3huVL1 | 65.09 | 77.02 |
| 1B6-huVH3huVL2 | 64.79 | 76.18 |
| 1B6-huVH3huVL3 | 64.72 | 75.37 |
| 1B6-huVH4huVL2 | 65.06 | 76.46 |
| 1B6-huVH4huVL3 | 65.40 | 76.09 |
| 1B6-huVH5huVL2 | 65.56 | 73.64 |
| 1B6-huVH5huVL3 | 65.68 | 73.01 |
| 1E7-mVHmVL | 65.19 | 74.57 |
| 1E7-huVH1huVL1 | 65.13 | 74.04 |
| 1E7-huVH1huVL2 | 65.19 | 72.48 |
| 1E7-huVH2huVL1 | 64.75 | 74.35 |
| 1E7-huVH2huVL2 | 72.45 | N/A |
| 1E7-huVH3huVL1 | 65.06 | 74.50 |
| 1E7-huVH3huVL2 | 64.69 | 73.42 |
| 1E7-huVH3huVL3 | 64.20 | 72.67 |
| 1E7-huVH4huVL2 | 65.53 | 73.11 |
| 1E7-huVH4huVL3 | 65.22 | 72.49 |
| 1E7-huVH5huVL2 | 71.28 | N/A |
| 1E7-huVH5huVL3 | 65.19 | 74.57 |

### Example 5 Affinity Evaluation of Humanized Antibodies by ELISA

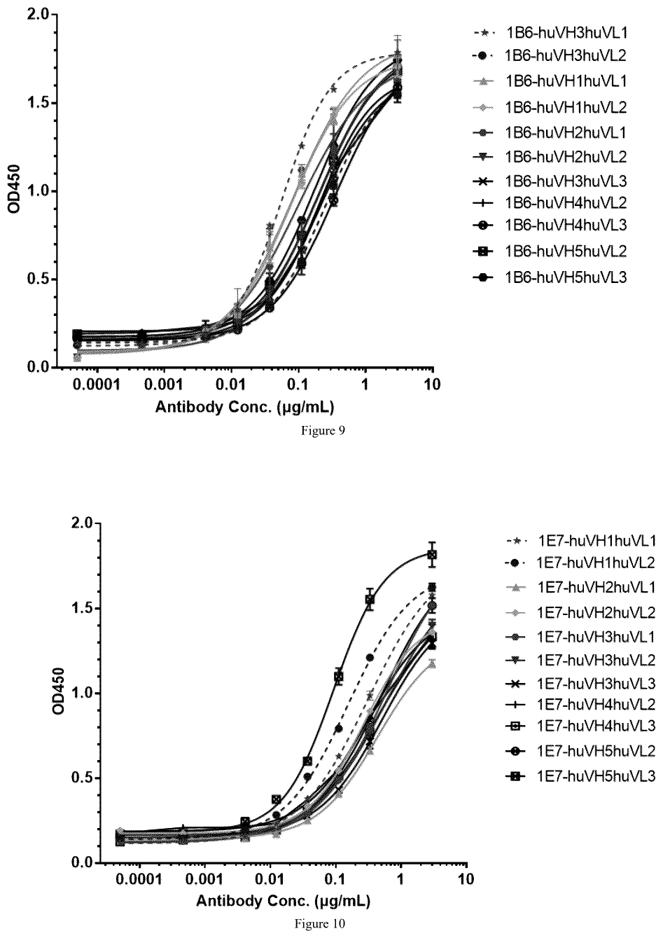

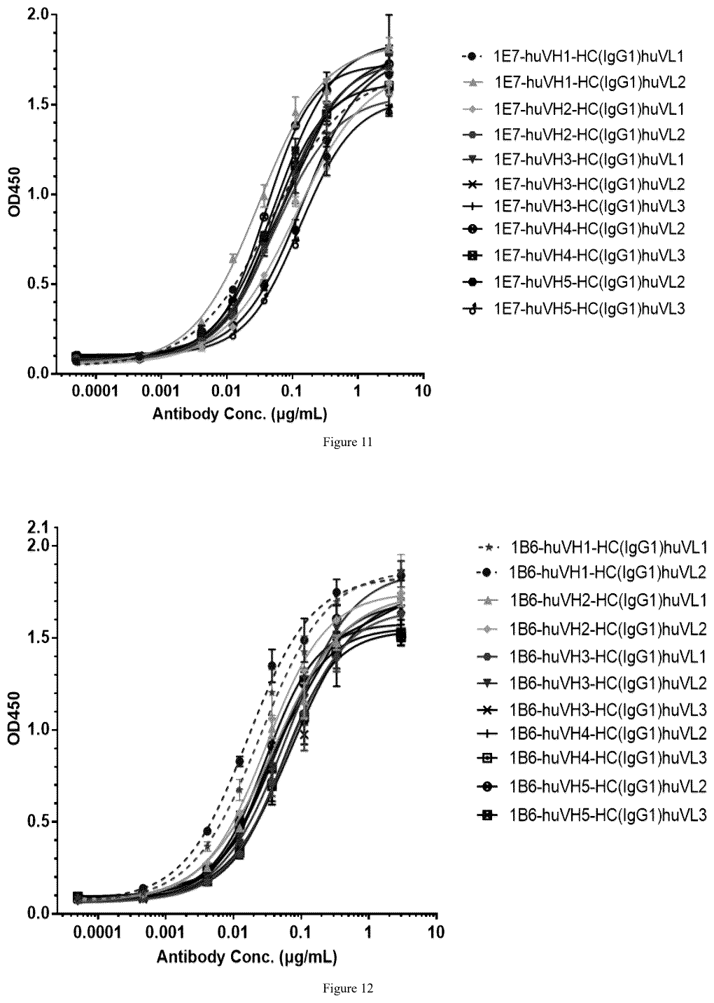

ELISA plates were coated with claudin-18.2 antigen (manufacturer: GenScript, batch number: R50092011) at 2 μg/mL, 30 μL per well, and incubated overnight at 4° C. The ELISA plates were washed three times with PBST and blocked with 5% PBS-milk for 2 h at room temperature. The ELISA plate was washed three times with PBST, and the diluted antibodies were added to the ELISA plate, 30 μL per well, and incubated at room temperature for 60 min. The plates were washed three times with PBST and incubated with anti-human IgG-Fc-HRP (abcam, ab97225) for 50 min at room temperature. The plate was washed 12 times with PBST, and TMB was added, 30 μL per well. The reaction was terminated with stop solution and OD450 was read. The specific experimental results are shown in FIGS. 9-12. The ELISA affinity EC50 was calculated. The specific experimental data are shown in Table 4, wherein the ELISA affinities of all antibodies were below 1.25 μg/mL, indicating that the humanized antibodies met the druggability requirements.

TABLE 4

| Sample name | EC50 (μg/mL) |
|---|---|
| 1E7-huVH1-HC (IgG1) huVL1 | 0.04788 |
| 1E7-huVH1-HC (IgG1) huVL2 | 0.03047 |
| 1E7-huVH2-HC (IgG1) huVL1 | 0.1114 |
| 1E7-huVH2-HC (IgG1) huVL2 | 0.05551 |
| 1E7-huVH3-HC (IgG1) huVL1 | 0.06634 |
| 1E7-huVH3-HC (IgG1) huVL2 | 0.05757 |
| 1E7-huVH3-HC (IgG1) huVL3 | 0.07069 |
| 1E7-huVH4-HC (IgG1) huVL2 | 0.03966 |
| 1E7-huVH4-HC (IgG1) huVL3 | 0.04997 |
| 1E7-huVH5-HC (IgG1) huVL2 | 0.1406 |
| 1E7-huVH5-HC (IgG1) huVL3 | 0.1301 |
| 1E7-huVH1huVL1 | 0.3093 |
| 1E7-huVH1huVL2 | 0.156 |
| 1E7-huVH2huVL1 | 0.4359 |
| 1E7-huVH2huVL2 | 0.2617 |
| 1E7-huVH3huVL1 | 0.6061 |
| 1E7-huVH3huVL2 | 0.5605 |
| 1E7-huVH3huVL3 | 0.5089 |
| 1E7-huVH4huVL2 | 0.3091 |
| 1E7-huVH4huVL3 | 0.4412 |
| 1E7-huVH5huVL2 | 0.4965 |
| 1E7-huVH5huVL3 | 0.09306 |
| 1B6-huVH1-HC (IgG1) huVL1 | 0.02478 |
| 1B6-huVH1-HC (IgG1) huVL2 | 0.01621 |
| 1B6-huVH2-HC (IgG1) huVL1 | 0.04025 |
| 1B6-huVH2-HC (IgG1) huVL2 | 0.03015 |
| 1B6-huVH3-HC (IgG1) huVL1 | 0.05919 |
| 1B6-huVH3-HC (IgG1) huVL2 | 0.06137 |
| 1B6-huVH3-HC (IgG1) huVL3 | 0.07251 |
| 1B6-huVH4-HC (IgG1) huVL2 | 0.04313 |
| 1B6-huVH4-HC (IgG1) huVL3 | 0.05188 |
| 1B6-huVH5-HC (IgG1) huVL2 | 0.02978 |
| 1B6-huVH5-HC (IgG1) huVL3 | 0.03496 |
| 1B6-huVH1huVL1 | 0.08392 |
| 1B6-huVH1huVL2 | 0.07751 |
| 1B6-huVH2huVL1 | 0.09779 |
| 1B6-huVH2huVL2 | 0.1918 |
| 1B6-huVH3huVL1 | 0.0569 |
| 1B6-huVH3huVL2 | 0.245 |
| 1B6-huVH3huVL3 | 0.2094 |
| 1B6-huVH4huVL2 | 0.2101 |
| 1B6-huVH4huVL3 | 0.3457 |
| 1B6-huVH5huVL2 | 0.2018 |
| 1B6-huVH5huVL3 | 0.1744 |

### Example 6 Affinity Kinetics Evaluation of Humanized Antibodies

The GATOR instrument and associated software were opened and the Kinetics experimental mode was selected. The affinity kinetics of the humanized antibody was evaluated according to the analysis program shown in Table 5. After data collection, Kinetics fitting was performed to calculate the affinity kinetic parameters. The correlation coefficient R² of all antibodies in the Global fitting mode was greater than 0.95, which met the system suitability requirements and the results were reliable. The experimental data of affinity kinetics testing of humanized antibodies are shown in Table 6, wherein all antibody KD values are between E−08 and E−11, indicating that humanized antibodies meet the druggability requirements, and some humanized antibodies have KD values comparable to or even better than mouse antibodies.

TABLE 5

| Assay step | Assay time (s) | | Sample |
|---|---|---|---|
| Baseline1 | 60 | sec | Q buffer |
| Loading | 120 | sec | 30 nM antibody in Q buffer |
| Baseline2 | 60 | sec | Q buffer |
| Association | 120 | sec | 2-fold diluted antigen from 2000 |
| | | | nM to 500 nM with Q buffer |
| Dissociation | 230 | sec | Q buffer |
| Regeneration | 25 | sec | Q buffer |

TABLE 6

| Sample name | KD (M) | ka (1/Ms) | kd (1/s) | R² |
|---|---|---|---|---|
| 1B6-mVH-HC (IgG1) mVL | 4.44E−08 | 1.78E+04 | 7.92E−04 | 0.992 |
| 1B6-huVH1-HC (IgG1) huVL1 | 6.33E−08 | 1.94E+04 | 1.23E−03 | 0.990 |
| 1B6-huVH1-HC (IgG1) huVL2 | 8.67E−08 | 1.97E+04 | 1.71E−03 | 0.990 |
| 1B6-huVH2-HC (IgG1) huVL1 | 9.84E−09 | 1.03E+04 | 1.01E−04 | 0.996 |
| 1B6-huVH2-HC (IgG1) huVL2 | 1.18E−08 | 1.10E+04 | 1.30E−04 | 0.996 |
| 1B6-huVH3-HC (IgG1) huVL1 | 7.65E−09 | 1.02E+04 | 7.83E−05 | 0.996 |
| 1B6-huVH3-HC (IgG1) huVL2 | 1.09E−08 | 1.11E+04 | 1.20E−04 | 0.996 |
| 1B6-huVH3-HC (IgG1) huVL3 | 9.64E−09 | 1.21E+04 | 1.16E−04 | 0.996 |
| 1B6-huVH4-HC (IgG1) huVL2 | 2.05E−08 | 1.01E+04 | 2.07E−04 | 0.996 |
| 1B6-huVH4-HC (IgG1) huVL3 | 4.39E−08 | 1.82E+04 | 7.99E−04 | 0.99 |
| 1B6-huVH5-HC (IgG1) huVL2 | 5.97E−08 | 2.16E+04 | 1.29E−03 | 0.987 |
| 1B6-huVH5-HC (IgG1) huVL3 | 7.68E−08 | 2.27E+04 | 1.75E−03 | 0.989 |
| 1B6-mVHmVL | 8.42E−08 | 2.23E+04 | 1.88E−03 | 0.989 |
| 1B6-huVH1huVL1 | 9.88E−08 | 2.24E+04 | 2.22E−03 | 0.989 |
| 1B6-huVH1huVL2 | 1.06E−08 | 1.55E+04 | 1.64E−04 | 0.996 |
| 1B6-huVH2huVL1 | 4.57E−09 | 1.39E+04 | 6.36E−05 | 0.995 |
| 1B6-huVH2huVL2 | 4.86E−09 | 1.39E+04 | 6.78E−05 | 0.996 |
| 1B6-huVH3huVL1 | 9.80E−10 | 1.30E+04 | 1.27E−05 | 0.996 |
| 1B6-huVH3huVL2 | 1.27E−08 | 9.48E+03 | 1.21E−04 | 0.997 |
| 1B6-huVH3huVL3 | 8.11E−09 | 1.06E+04 | 8.56E−05 | 0.996 |
| 1B6-huVH4huVL2 | 7.06E−09 | 9.99E+03 | 7.06E−05 | 0.996 |
| 1B6-huVH4huVL3 | 7.24E−09 | 1.11E+04 | 8.07E−05 | 0.996 |
| 1B6-huVH5huVL2 | 1.06E−08 | 1.36E+04 | 1.44E−04 | 0.996 |
| 1B6-huVH5huVL3 | 7.51E−09 | 1.30E+04 | 9.78E−05 | 0.996 |
| 1E7-mVHmVL | 2.66E−09 | 1.02E+04 | 2.71E−05 | 0.995 |
| 1E7-huVH1huVL1 | 3.58E−09 | 1.06E+04 | 3.81E−05 | 0.995 |
| 1E7-huVH1huVL2 | 5.08E−09 | 1.09E+04 | 5.56E−05 | 0.995 |
| 1E7-huVH2huVL1 | 2.77E−10 | 9.55E+03 | 2.65E−06 | 0.995 |
| 1E7-huVH2huVL2 | 2.16E−08 | 1.24E+04 | 2.69E−04 | 0.992 |
| 1E7-huVH3huVL1 | 1.20E−08 | 1.10E+04 | 1.32E−04 | 0.995 |
| 1E7-huVH3huVL2 | 2.02E−09 | 1.03E+04 | 2.08E−05 | 0.995 |
| 1E7-huVH3huVL3 | 1.35E−08 | 1.10E+04 | 1.49E−04 | 0.995 |
| 1E7-huVH4huVL2 | 4.60E−09 | 1.06E+04 | 4.86E−05 | 0.995 |
| 1E7-huVH4huVL3 | 6.19E−09 | 1.01E+04 | 6.27E−05 | 0.995 |
| 1E7-huVH5huVL2 | 5.24E−09 | 1.01E+04 | 5.29E−05 | 0.996 |
| 1E7-huVH5huVL3 | 3.21E−09 | 9.61E+03 | 3.09E−05 | 0.995 |
| 1E7-mVH-HC (IgG1) mVL | 3.82E−09 | 1.17E+04 | 4.48E−05 | 0.995 |
| 1E7-huVH1-HC (IgG1) huVL1 | 4.90E−09 | 1.22E+04 | 5.97E−05 | 0.995 |
| 1E7-huVH1-HC (IgG1) huVL2 | 4.85E−09 | 1.21E+04 | 5.87E−05 | 0.995 |
| 1E7-huVH2-HC (IgG1) huVL1 | 1.82E−09 | 1.09E+04 | 1.97E−05 | 0.995 |
| 1E7-huVH2-HC (IgG1) huVL2 | 2.89E−09 | 1.10E+04 | 3.18E−05 | 0.995 |
| 1E7-huVH3-HC (IgG1) huVL1 | 3.50E−10 | 1.03E+04 | 3.60E−06 | 0.995 |
| 1E7-huVH3-HC (IgG1) huVL2 | 2.89E−10 | 1.02E+04 | 2.94E−06 | 0.995 |
| 1E7-huVH3-HC (IgG1) huVL3 | 1.16E−08 | 1.13E+04 | 1.32E−04 | 0.994 |
| 1E7-huVH4-HC (IgG1) huVL2 | 1.07E−08 | 1.18E+04 | 1.26E−04 | 0.994 |
| 1E7-huVH4-HC (IgG1) huVL3 | 2.64E−09 | 1.13E+04 | 2.99E−05 | 0.995 |
| 1E7-huVH5-HC (IgG1) huVL2 | 1.31E−08 | 1.28E+04 | 1.67E−04 | 0.993 |
| 1E7-huVH5-HC (IgG1) huVL3 | 9.29E−10 | 1.06E+04 | 9.83E−06 | 0.995 |

### Example 7 Evaluation of ADCC Activity of Humanized Antibodies

KATOIII-Claudin18.2 cells (target cells) were cultured in RPMI1640 (Gibco, 22400-089) medium containing 10% FBS (Gibco, 10099-141C) and 0.5 μg/mL puromycin (Gibco, A11138-03). The cells were collected in the logarithmic growth phase, washed thoroughly with PBS (Hyclone, SHS30256.01), and resuspended in RPMI1640 medium containing 1% FBS and 1% double antibody (Hyclone, SV30010). The cell concentration was adjusted to 4×10⁵ cells per ml and 5000 cells were plated in each well of a white bottom opaque 384-well microplate (Corning, 3570). The humanized antibody was diluted in a 4-fold gradient from 3.33 μg/mL to prepare 10 concentrations, and 25 μL per well was added to the cell wells. Jurkat-NFAT-Luc2/CD16 (effector cells) were cultured in RPMI1640 medium containing 10% FBS and 1% double antibody. The cells were collected in the logarithmic growth phase, washed thoroughly with PBS, and resuspended in RPMI1640 medium containing 1% FBS and 1% double antibody. The cell concentration was adjusted to 2×10⁶ cells per ml and 20,000 cells per well were added to the cell wells. The 384-well microplate was placed in a 7° C., 5% CO₂ incubator for 12 h, 30 μL One-Glo™ Luciferase detection reagent (Promega, E6120) was added to each well, and the optical luminescence signal was read using a multi-function microplate reader (SpectraMax).

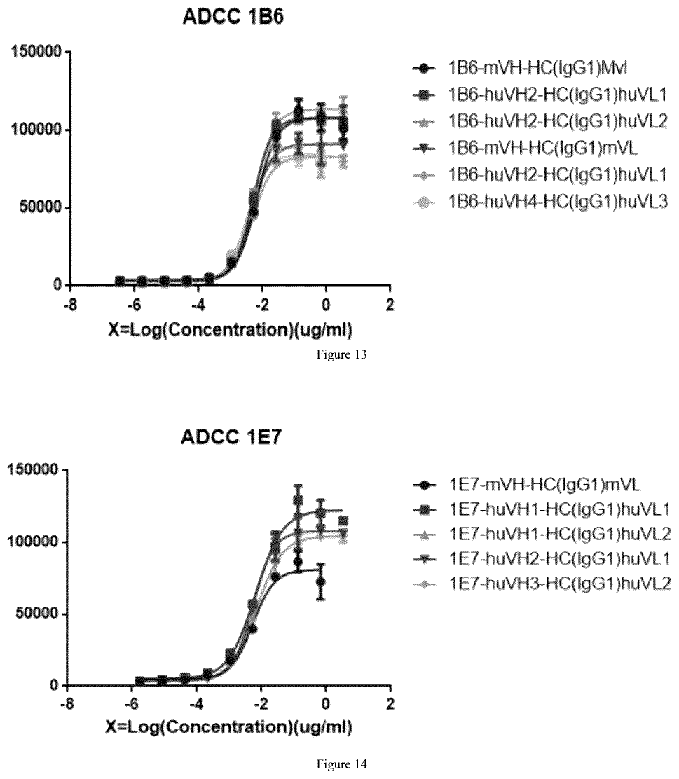

The experimental results of ADCC activity evaluation of 1B6 partially humanized antibody and murine antibody are shown in FIG. 13, wherein the ADCC activity EC50 of the humanized antibody is not much different from that of the murine antibody, and the highest value of ADCC activity of the partially humanized antibody is comparable to that of the chimeric antibody, or even better. The results of ADCC activity evaluation of 1E7 partially humanized antibody and chimeric antibody are shown in FIG. 14, wherein the ADCC activity EC50 of the humanized antibody is not much different from that of the chimeric antibody, and the highest value of ADCC activity of the partially humanized antibody is comparable to that of the chimeric antibody, or even better.

### Example 8 Evaluation of CDC Activity of Humanized Antibodies

KATOIII-claudin18.2 cells were cultured in RPMI1640 (Gibco, 22400-089) medium containing 10% FBS (Gibco, 10099-141C) and 0.5 μg/mL puromycin (Gibco, A11138-03). The cells were collected in the logarithmic growth phase, washed thoroughly with PBS (Hyclone, SHS30256.01), and resuspended in RPMI1640 medium containing 2.5% human serum complement components (Quidel, A113) and 1% double antibody (Hyclone, SV30010). The cell concentration was adjusted to 3×10⁵ cells per ml and 100 μL was plated per well in a 96-well cell culture plate (Corning, 3599). The humanized antibody was diluted 4-fold from 270 μg/mL using RPMI1640 medium containing 2.5% human serum complement components and 1% double antibody to prepare 10 concentrations, and 100 μL per well was added to the cell wells. The 384-well microplate was placed in a 37° C., 5% CO₂ incubator for 4 h, 50 μL of 40% CCK-8 detection reagent (Japan Tongren, CK04) was added to each well, and the incubation continued for 3 h. The OD450 was read using a multi-function microplate reader (SpectraMax).

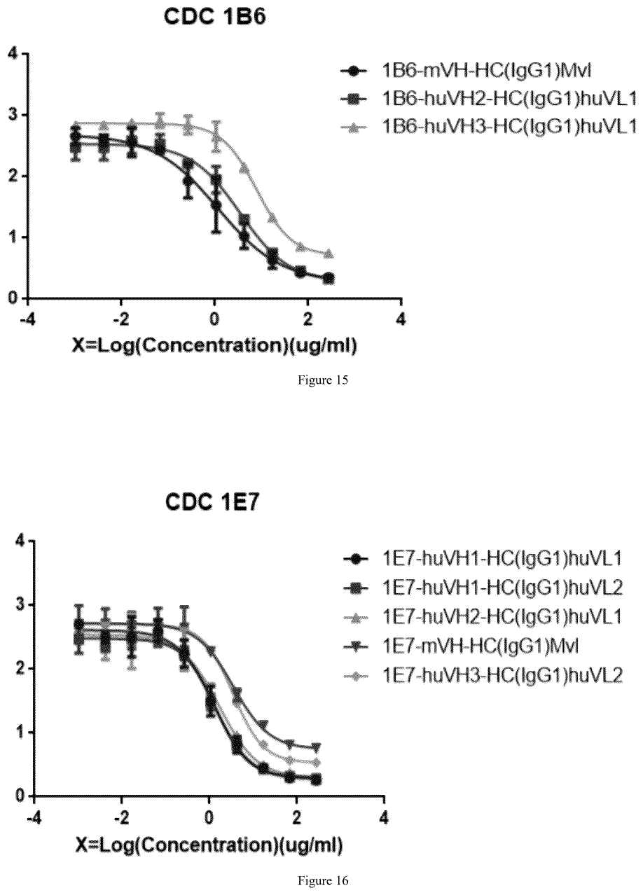

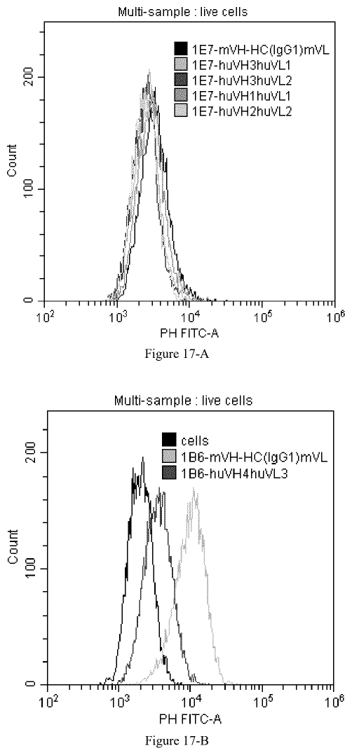

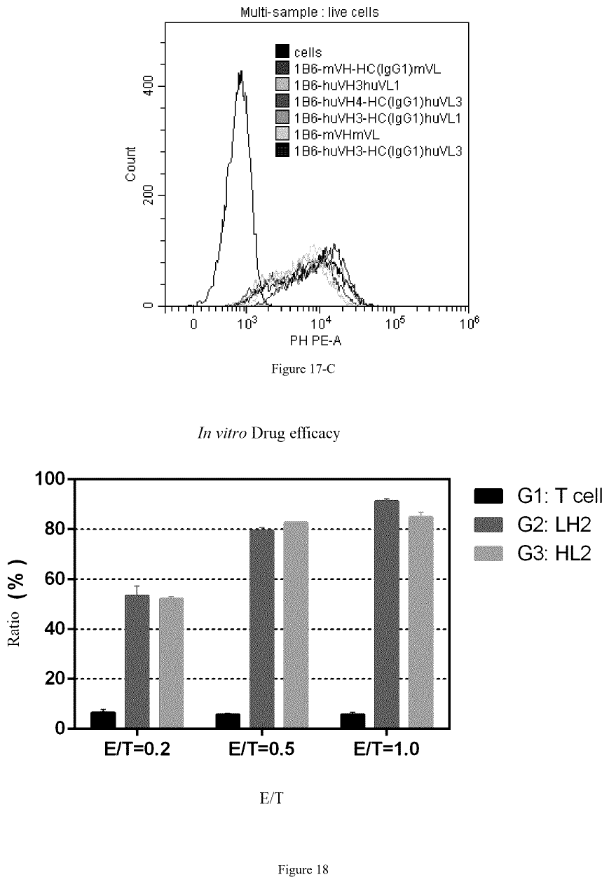

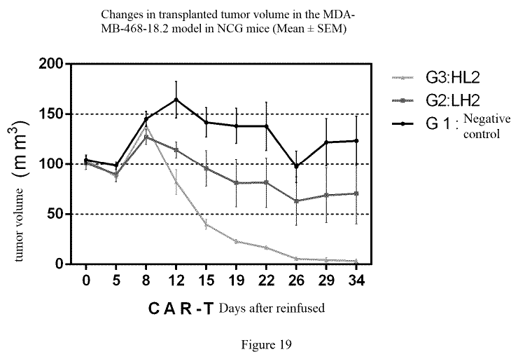

The results of CDC activity evaluation of 1B6 partially humanized antibody and chimeric antibody are shown in FIG. 15, wherein the CDC activity EC50 of the humanized antibody is not much different from that of the chimeric antibody, and the highest value of CDC activity of the partially humanized antibody is comparable to that of the chimeric antibody, or even better. The results of CDC activity evaluation of 1E7 partially humanized antibody and chimeric antibody are shown in FIG. 16, wherein the CDC activity EC50 of the humanized antibody is not much different from that of the chimeric antibody, and the highest value of CDC activity of the partially humanized antibody is comparable to that of the chimeric antibody, or even better.

### Example 9 Evaluation of Internalization of Humanized Claudin18.2 Antibody Using FACS with Zenon™ pHrodo™ iFL IgG Labeling Reagent

Claudin18.2 stable expression cell line A20˜18.2 (target cells) was constructed and cultured in 1640 complete medium (Gibco, 22400-089) containing 10% FBS (Gibco, 10099-141C), 0.05 mM β-mercaptoethanol (Aladdin, M301574) and 1 μg/ml puromycin (Gibco, A11138-03). When the cells grew to about 80% of the entire culture dish, the cells were centrifuged and the viability was measured with trypan blue to ensure that the cell viability was above 95%. The cells were counted using an automatic cell counter and the cell density was adjusted to 2*10\hat{ }6 cells/mL. The antibody to be tested was prepared using 1640 complete medium, and the final concentration of the antibody to be tested was 2 μg/mL. Zenon™ pHrodo™ iFL IgG Labeling Reagent (Invitrogen, Z25611, Z25612) was prepared using 1640 complete medium at a final concentration of 40 nM. The antibody to be tested of 25 μL and Zenon™ pHrodo™ iFL IgG Labeling Reagent of 25 μL were added to a 96-well plate, and the 96-well plate was incubated for 5 min at room temperature. To each well was added A20˜18.2 cells in 50 μL, with a cell volume of 1*10\hat{ }5 cells per well. The 96-well plate was placed in an incubator at 37° C. and 5% CO₂ for 4 h. The remaining antibodies or reagents were washed away with PBS. The antibody internalization was detected by flow cytometry, and the proportion of cells that underwent internalization was counted.

The results are shown in Table 7 and FIGS. 17A-C. Claudin18.2 murine monoclonal antibody 1B6 underwent obvious antibody internalization, with the maximum internalized cell ratio reaching 92.83%. Under the same conditions, after humanization of 1B6 antibody, the internalization ratio of the antibody decreased to varying degrees, and the degree of reduction was related to the IgG subtype of the antibody and the degree of humanization. The murine monoclonal antibody 1E7 showed no obvious antibody internalization, and antibody internalization still did not occur after humanization.

TABLE 7
Fluorescence intensity of internalization detection
of Claudin18.2 humanized monoclonal antibody
and the proportion of internalized cells

| | | | The |
|---|---|---|---|
| | Antibody | The | proportion of |
| | concen- | fluorescence | internalized |
| | tration | intensity | cells |
| Antibody | (μg/mL) | (MFI) | (%) |
| cells | 0 | 2044 | 0.08% |
| 1B6-mVH-HC (IgG1) mVL | 2 | 10504 | 83.75% |
| 1B6-huVH4huVL3 | 2 | 3817 | 10.50% |
| 1E7-mVH-HC (IgG1) mVL | 2 | 3485 | 7.07% |
| 1E7-huVH3huVL1 | 2 | 2754 | 2.07% |
| 1E7-huVH3huVL2 | 2 | 2595 | 1.44% |
| 1E7-huVH1huVL1 | 2 | 3078 | 3.86% |
| 1E7-huVH2huVL2 | 2 | 2643 | 1.54% |
| cells | 0 | 844 | 0.02% |
| 1B6-mVH-HC (IgG1) mVL | 2 | 11592 | 92.83% |
| 1B6-huVH3huVL1 | 2 | 6303 | 72.40% |
| 1B6-huVH4-HC (IgG1) huVL3 | 2 | 6993 | 73.78% |
| 1B6-huVH3-HC (IgG1) huVL1 | 2 | 10035 | 81.59% |
| 1B6-mVHmVL | 2 | 7446 | 84.14% |
| 1B6-huVH3-HC (IgG1) huVL3 | 2 | 9698 | 80.29% |

### Example 10 Pharmacokinetic Evaluation of Humanized Antibodies in Mice

Eighteen male Babl/c mice were randomly divided into 6 groups, with 3 mice in each group. They were subcutaneously injected with 2 mg/kg of the following: (1) chimeric antibody 1B6 and its humanized antibodies P04891 (1B6-huVH4huVL3) and P04887 (1B6-huVH3huVL1); (2) chimeric antibody 1E7 and its humanized antibodies P04898 (1E7-huVH2huVL2) and P04902 (1E7-huVH4huVL2). Blood was collected at 0.25, 2, 7, 24, 48, 72, 120, 168, 240, 336, 456, and 504 h after administration, and plasma was separated (anticoagulated with EDTA-K2). The concentration of chimeric antibodies and humanized antibodies in each sample was analyzed by FACS. Gradient concentrations of test articles and plasma samples were added to A20 cells stably expressing Claudin18.2, and then FITC-conjugated secondary antibodies (sheep anti-human IgG) were added to bind to the test sample. The cell surface fluorescence intensity was detected by flow cytometry and a fitting curve of antibody concentration-fluorescence intensity was drawn to calculate the drug concentration in plasma.

Pharmacokinetic parameters were calculated based on plasma drug concentrations. The results of main PK parameters were shown in Table 8. The results showed that after subcutaneous injection of chimeric antibody murine 1B6 monoclonal antibody (1B6-mVHmVL) and its humanized antibody P04891 (1B6-huVH4huVL3), P04887 (1B6-huVH3huVL1) into Babl/c mice, the average plasma half-lives were 70 h, 76 h and 160 h, respectively; the average plasma half-lives of chimeric antibody murine 1E7 monoclonal antibody (1E7-mVHmVL) and its humanized antibody P04898 (1E7-huVH2huVL2), P04902 (1E7-huVH4huVL2) were 111 h, 177 h and 196 h, respectively. Compared with their respective chimeric antibodies, humanized antibodies can significantly extend the half-life of the antibody in the body.

TABLE 8
Half-life of Claudin18.2 chimeric mAb
and its humanized mAb in mice (h)

T½ (h)

| Species | Analyte | Group | Dosage | Mean | SD |
|---|---|---|---|---|---|
| Babl/c | 1B6 | sc1 | 2.0 mg/kg | 69.9 | 12 |
| Mouse | P04891 | sc2 | 2.0 mg/kg | 75.9 | 50 |
| | P04887 | sc3 | 2.0 mg/kg | 160 | 59 |
| | 1E7 | sc4 | 2.0 mg/kg | 111 | 34 |
| | P04898 | sc5 | 2.0 mg/kg | 177 | 110 |
| | P04902 | sc6 | 2.0 mg/kg | 196 | 32 |

### Example 11 Study on the In Vitro Effects of Humanized Claudin18.2 Antibody CAR-T

MDA-MB-468-18.2 cells stably expressing Claudin18.2-ΔC5 were cultured in DMEM complete medium (containing 10% FBS and 1% Pen/Strep double antibody) and placed in a 37° C., 5% carbon dioxide incubator.

When the tumor cells grew to the required number, the cells were taken in the logarithmic growth phase. The original medium was discarded, and the mixture was trypsinized for 3 minutes. Then, the digestion was terminated with DMEM medium containing 10% FBS, the cells were collected and centrifuged at 400 rpm for 5 min. After cell counting, cells were diluted with 10% FBS DMEM and plated on a flat-bottom 96-well plate, 200 μL per well, and 2E+04 cells per well were set. After the tumor cells adhered for 6 h, the effector-target ratio E/T was set to 0.2, 0.5, and 1.0, that is, the number of CAR-positive cells corresponding to each well was 4E+03, 1E+04, and 2E+04, respectively, and then CART cells were co-incubated with target cells. CTS complete culture was used for killing incubation, and the culture system for each well was 200 μL. Three replicate wells were set for each group, and 3 parallel wells containing only T cells were set for each effector-target ratio.

After co-incubation and plating, the cells were cultured in a 37° C., 5% carbon dioxide incubator. After culturing for 20 h, the killing assay was performed using the LDH method, and the absorbance was measured at 490 nm.

$$\begin{matrix}Data processing : Positive control ( P S ) = ( target + 1 ysis ) - ( target + NC ) \\ Experimental group killing ( EFF ) = ( E / T = n ) - ( only T , E / T = n ) \\ Cytotoxicity = 100 \% * ( EFF ) / ( PS )\end{matrix}$$

The results were as shown in FIG. 18. Wherein, G1: negative control group, G2: LH2 group, G3: HL2 group, a total of 3 groups. G1 is T cells; G2 and G3 groups are T cells expressing CAR targeting Claudin18.2 molecules, respectively. The VH and VL directions of the scFv sequences in the CAR molecules of G2 and G3 are opposite, wherein the G2 group is connected with VL-GS linker-VH, and the G3 group is connected with VH-GS linker-VL, both of which are humanized 1E7 sequences (VL is IE7-huVL2, the amino acid sequence is shown in SEQ ID NO: 66, and VH is IE7-huVH4, the amino acid sequence is shown in SEQ ID NO: 71).

It can be seen from the figure that the in vitro killing effects of the G2 and G3 groups are significantly better than that of the G1 control group.

### Example 12 Study on the In Vivo Effects of Humanized Claudin18.2 Antibody CAR-T

MDA-MB-468-18.2 cells stably expressing Claudin18.2-ΔC5 were cultured in DMEM complete medium (containing 10% FBS and 1% Pen/Strep double antibody) and placed in a 37° C., 5% carbon dioxide incubator. When the cells grew to the required number, the cells were taken in the logarithmic growth phase. The original medium was discarded, and ½ volume of PBS was added for washing, and the PBS was discarded. Then an appropriate amount of 0.05% trypsin was added for digestion for 3 minutes. The digestion was then terminated with complete DMEM medium. The cells were collected and centrifuged at 1000 rpm for 5 min. 0.5 ml of culture medium was used to extract the genome and the mycoplasma test was negative by PCR. After cell counting, the cell density was adjusted to 5E+07/mL with a serum-free DMEM medium and Matrigel mixture (at a ratio of 1:1). Fixed NCG female mice were grabbed, and the cell suspension was injected into the right back of the mouse subcutaneously with 100 μL/mouse.

In the MDA-MB-468-18.2 model, when the tumor grew to about 100 mm³, the animals were divided into groups and given medication. There were three groups in total: G1: Vehicle group, G2: LH2 group, and G3: HL2 group. Wherein G1 was the negative control group and was injected with PBS; G2 and G3 respectively expressed CAR targeting Claudin18.2 molecules. The VH and VL directions of the scFv sequences of the CAR molecules of G2 and G3 were opposite. The G2 group was connected with VL-GS linker-VH, and the G3 group was connected with VH-GS linker-VL. Both the VH and VL sequences were obtained after humanization of the original 1E7 sequence (wherein VL is IE7-huVL2, with an amino acid sequence as shown in SEQ ID NO: 66, and VH is IE7-huVH4, with an amino acid sequence as shown in SEQ ID NO: 71).

LH2 CAR-T and HL2 CAR-T cells were collected, and the cell density was adjusted to 1.25E+07/ml of CAR-positive cells with PBS solution. Each mouse was administered 200 μL of cell solution through the tail vein, i.e., 2.5E+06 CAR-positive cells. The G1 group was injected with 200 μL of PBS through the tail vein. After the end of administration, the length (L) and width (W) of the tumor were measured twice a week, and the tumor volume (V) was calculated, V=L*W*W*0.5; the tumor growth inhibition rate (TGI) was then calculated. If T_{X}>T₀, TGI=[1−T_{X}/C_{X}]*100%; if T_{X}<T₀, TGI=[1−(T_{X}−T₀)/T₀]*100%; wherein T_{X} and C_{X} are the tumor volumes of the experimental group and the control group on the measurement day, and T₀ and C₀ are the tumor volumes of the experimental group and the control group on the day of administration. Statistical analysis was performed using GraphPad Prism 6, and the results are shown in FIG. 19.

The results show that both LH2 CAR-T and HL2 CAR-T can significantly inhibit tumor growth after intravenous administration in the transplanted tumor model established in NCG mice. At the end of the experiment, the tumor growth inhibition rate of LH2 CAR-T was 131%, and that of HL2 CAR-T was 197%, with both showing significant statistical differences (P<0.01).

Reference throughout this specification to “an embodiment,” “some embodiments,” “one embodiment”, “another example,” “an example,” “a specific example,” or “some examples,” means that a particular feature, structure, material, or characteristic described in connection with the embodiment or example is included in at least one embodiment or example of the present disclosure. Thus, the appearances of the above terms throughout this specification are not necessarily referring to the same embodiment or example of the present disclosure. Furthermore, the particular features, structures, materials, or characteristics may be combined in any suitable manner in one or more embodiments or examples. In addition, those skilled in the art can integrate and combine different embodiments, examples or the features of them as long as they are not contradictory to one another.

Although explanatory embodiments have been shown and described, it would be appreciated by those skilled in the art that the above embodiments cannot be construed to limit the present disclosure, and changes, alternatives, and modifications can be made in the embodiments without departing from spirit, principles and scope of the present disclosure.

1.-36. (canceled)

37. An antibody or antigen-binding fragment capable of specifically recognizing Claudin18.2, wherein the antibody comprises at least one CDR sequence selected from the following or an amino acid sequence having at least 90% identity with it:
the CDR sequences of light chain variable region:
(SEQ ID NO: 1)
X₁SSQSLFNSGNQKNYLT,
(SEQ ID NO: 2)
WASTRES,
(SEQ ID NO: 3)
QNDYSYPLT,
(SEQ ID NO: 7)
X₄SSQSLLNSGNQKNYLT,
(SEQ ID NO: 8)
WASTRES,
and
(SEQ ID NO: 9)
QNDYSYPFT;
the CDR sequences of heavy chain variable region:
(SEQ ID NO: 4)
DYNMH,
(SEQ ID NO: 5)
YINPNNGGTSYX₂QKFX₃G,
(SEQ ID NO: 6)
TRYLAV,
(SEQ ID NO: 10)
SYWMH,
(SEQ ID NO: 11)
MIHPNSGSTNYX₅X₆KFX₇X₈,
and
(SEQ ID NO: 12)
RYYGSISPDY;
wherein, X₁ is K or R, X₂ is N or S, X₃ is K or Q, X₄ is K or R, X₅ is N or A, X₆ is E or Q, X₇ is K or Q, X₈ is S or G;
wherein, the antibody or antigen-binding fragment thereof has a humanization modification.

38. The antibody or antigen-binding fragment thereof according to claim 37, wherein the antibody comprises:
the light chain variable region CDR1, CDR2, and CDR3 having sequences shown in SEQ ID NO: 1, 2 and 3 respectively or having at least 90% identity with SEQ ID NO: 1, 2 and 3; or
the light chain variable region CDR1, CDR2, and CDR3 having sequences shown in SEQ ID NO: 7, 8 and 9 respectively or having at least 90% identity with SEQ ID NO: 7, 8 and 9; or
the heavy chain variable region CDR1, CDR2, and CDR3 having sequences shown in SEQ ID NO: 4, 5 and 6 respectively or having at least 90% identity with SEQ ID NO: 4, 5 and 6; or
the heavy chain variable region CDR1, CDR2, and CDR3 having sequences shown in SEQ ID NO: 10, 11 and 12 respectively or having at least 90% identity with SEQ ID NO: 10, 11 and 12.

39. The antibody or antigen-binding fragment thereof according to claim 37, the antibody comprises at least one of a heavy chain framework region sequence and a light chain framework region sequence, wherein at least a part of at least one of the heavy chain framework region sequence and the light chain framework region sequence is derived from at least one of a human antibody or a mutant thereof.

40. The antibody or antigen-binding fragment thereof according to claim 37, wherein the antibody comprises:
the light chain framework region FRL1, FRL2, FRL3, and FRL4 having sequences shown in SEQ ID NO: 13-16 respectively,
(SEQ ID NO: 13)
DIX₉MTQSPSSLX₁₀X₁₁X₁₂X₁₃GX₁₄X₁₅VTX₁₆SX₁₇C,
(SEQ ID NO: 14)
WYQQKPGX₁₈X₁₉PKLLIY,
(SEQ ID NO: 16)
FGX₂₇GTKLEX₂₈K;
(SEQ ID NO: 15)
GVPX₂₀DRFX₂₁GSGSGTDFTLTISSX₂₂QX₂₃EDX₂₄AX₂₅YX₂₆C,
or
the light chain framework region FRL1, FRL2, FRL3, and FRL4 having sequences shown in SEQ ID NO: 21-24 respectively,
(SEQ ID NO: 21)
DIX₅₃MTQSPSSLSX₅₄X₅₅AX₅₆GX₅₇X₅₈VTX₅₉X₆₀C,
(SEQ ID NO: 22)
WYQQKPGX₆₁X₆₂PKLLIY,
(SEQ ID NO: 23)
GVPX₆₃RFX₆₄GSGSGTDFTLTISSX₆₅QX₆₆EDX₆₇AX₆₈YYC,
(SEQ ID NO: 24)
FGX₆₉GTKLEIK,
wherein, X₉ is V or Q, X₁₀ is S or T, X₁₁ is V or A, X₁₂ is T or S, X₁₃ is A or V, X₁₄ is E or D, X₁₅ is K or R, X₁₆ is M or I, X₁₇ is S or T, X₁₈ is Q or K, X₁₉ is P or A, X₂₀ is D or S, X₂₁ is T or S, X₂₂ is V or L, X₂₃ is A or P, X₂₄ is L or F, X₂₅ is V or T, X₂₆ is F or Y, X₂₇ is A or G, X₂₈ is L or I, X₅₃ is V or Q, X₅₄ is V or A, X₅₅ is T or S, X₅₆ is A or V, X₅₇ is E or D, X₅₈ is K or R, X₅₉ is M or I, X₆₀ is S or T, X₆₁ is Q or K, X₆₂ is P or A, X₆₃ is D or S, X₆₄ is T or S, X₆₅ is V or L, X₆₆ is A or P, X₆₇ is L or F, X₆₈ is V or T, X₆₉ is S or G;
the condition is that when the antibody comprises: the light chain variable region CDR1, CDR2, and CDR3 sequences shown in KSSQSLFNSGNQKNYLT (SEQ ID NO: 29), WASTRES (SEQ ID NO: 30), and QNDYSYPLT (SEQ ID NO: 31), respectively, X₉ is V, X₁₀ is T, X₁₁ is V, X₁₂ is T, X₁₃ is A, X₁₄ is E, X₁₅ is K, X₁₆ is M, X₁₇ is S, X₁₈ is Q, X₁₉ is P, X₂₀ is D, X₂₁ is T, X₂₂ is V, X₂₃ is A, X₂₄ is L, X₂₅ is V, X₂₆ is F, X₂₇ is A, and X₂₈ is L, which are not established at the same time;
when the antibody comprises: the light chain variable region CDR1, CDR2, and CDR3 sequences shown in KSSQSLLNSGNQKNYLT (SEQ ID NO: 35), WASTRES (SEQ ID NO: 36), QNDYSYPFT (SEQ ID NO: 37), respectively, X₅₃ is V, X₅₄ is V, X₅₅ is T, X₅₆ is A, X₅₇ is E, X₅₈ is K, X₅₉ is M, X₆₀ is S, X₆₁ is Q, X₆₂ is P, X₆₃ is D, X₆₄ is T, X₆₅ is V, X₆₆ is A, X₆₇ is L, X₆₈ is V, and X₆₉ is S, which are not established at the same time.

41. The antibody or antigen-binding fragment thereof according to claim 37, wherein the antibody comprises:
the light chain framework region FRL1, FRL2, FRL3, and FRL4 having sequences shown in SEQ ID NO: 41-44 respectively,
(SEQ ID NO: 44)
FGAGTKLELK;
(SEQ ID NO: 41)
DIX₉₀MTQSPSSLSX₉₁TAGEKVTX₉₂SC,
(SEQ ID NO: 42)
WYQQKPGQPPKLLIY,
(SEQ ID NO: 43)
GVPX₉₃DRFTGSGSGTDFTLTISSX₉₄QAEDLAVYX₉₅C,
the light chain framework region FRL1, FRL2, FRL3, and FRL4 having sequences shown in SEQ ID NO: 49-52 respectively,
(SEQ ID NO: 49)
DIX₁₀₇MTQSPSSLSX₁₀₈TAGEKVTX₁₀₉SC,
(SEQ ID NO: 50)
WYQQKPGX₁₁₀X₁₁₁PKLLIY,
(SEQ ID NO: 51)
GVPX₁₁₂RFTGSGSGTDFTLTISSX₁₁₃QAEDLAVYYC,
(SEQ ID NO: 52)
FGSGTKLEIK,
wherein, X₉₀ is V or Q, X₉₁ is V or A, X₉₂ is M or I, X₉₃ is D or S, X₉₄ is V or L, X₉₅ is F or Y, X₁₀₇ is V or Q, X₁₀₈ is V or A, X₁₀₉ is M or I, X₁₁₀ is Q or K, X₁₁₁ is P or A, X₁₁₂ is D or S, and X₁₁₃ is V or L.

42. The antibody or antigen-binding fragment thereof according to claim 37, wherein the antibody comprises:
the heavy chain framework region FRH1, FRH2, FRH3, and FRH4 having sequences shown in SEQ ID NO: 17-20 respectively,
(SEQ ID NO: 17)
X₂₉VQLX₃₀QSGX₃₁EX₃₂X₃₃X₃₄PGASVKX₃₅SCKASGYTFT,
(SEQ ID NO: 18)
WVX₃₆QX₃₇X₃₈GX₃₉X₄₀LEWX₄₁G,
(SEQ ID NO: 19)
X₄₂X₄₃TX₄₄TX₄₅X₄₆X₄₇SX₄₈STAYMELX₄₉SLX₅₀SEDX₅₁AVYYCVT,
(SEQ ID NO: 20)
WGX₅₂GTTVTVSS;
or
the heavy chain framework region FRH1, FRH2, FRH3, and FRH4 having sequences shown in SEQ ID NO: 25-28 respectively,
(SEQ ID NO: 25)
QVQLX₇₀QX₇₁GX₇₂EX₇₃X₇₄KPGASVKX₇₅SCKASGYTFT,
(SEQ ID NO: 26)
WVX₇₆QX₇₇PGQGLEWX₇₈G,
(SEQ ID NO: 27)
X₇₉X₈₀X₈₁TX₈₂TVDX₈₃SX₈₄STX₈₅YMX₈₆LSSLX₈₇SEDX₈₈AVYYCAR,
(SEQ ID NO: 28)
WGQGTTX₈₉TVSS,
wherein, X₂₉ is E or Q, X₃₀ is Q or V, X₃₁ is P or A, X₃₂ is L or V, X₃₃ is V or K, X₃₄ is R or K, X₃₅ is M or V, X₃₆ is K or R, X₃₇ is S or A, X₃₈ is H or P, X₃₉ is K or Q, X₄₀ is S or R, X₄₁ is I or M, X₄₂ is K or R, X₄₃ is A or V, X₄₄ is L or I, X₄₅ is V or R, X₄₆ is N or D, X₄₇ is K or T, X₄₈ is S or A, X₄₉ is R or S, X₅₀ is T or R, X₅₁ is S or T, X₅₂ is T or Q, X₇₀ is Q or V, X₇₁ is P or S, X₇₂ is S or A, X₇₃ is L or V, X₇₄ is V or K, X₇₅ is L or V, X₇₆ is K or R, X₇₇ is R or A, X₇₈ is I or M, X₇₉ is S or G, X₈₀ is R or K, X₈₁ is A or V, X₈₂ is L or M, X₈₃ is K or T, X₈₄ is S or T, X₈₅ is A or V, X₈₆ is Q or E, X₈₇ is T or R, X₈₈ is S or T, X₈₉ is L or V;
the condition is that when the antibody comprises: the heavy chain variable region CDR1, CDR2, and CDR3 sequences shown in DYNMH (SEQ ID NO: 32), YINPNNGGTSYNQKFKG (SEQ ID NO: 33), TRYLAV (SEQ ID NO: 34), respectively, X₂₉ is E, X₃₀ is Q, X₃₁ is P, X₃₂ is L, X₃₃ is V, X₃₄ is R, X₃₅ is M, X₃₆ is K, X₃₇ is S, X₃₈ is H, X₃₉ is K, X₄₀ is S, X₄₁ is I, X₄₂ is K, X₄₃ is A, X₄₄ is L, X₄₅ is V, X₄₆ is N, X₄₇ is K, X₄₈ is S, X₄₉ is R, X₅₀ is T, X₅₁ is S, X₅₂ is T, which are not established at the same time;
when the antibody comprises: the heavy chain variable region CDR1, CDR2, and CDR3 sequences shown in SYWMH (SEQ ID NO: 38), MIHPNSGSTNYNEKFKS (SEQ ID NO: 39), RYYGSISPDY (SEQ ID NO: 40), respectively, X₇₀ is Q, X₇₁ is P, X₇₂ is S, X₇₃ is L, X₇₄ is V, X₇₅ is L, X₇₆ is K, X₇₇ is R, X₇₈ is I, X₇₉ is S, X₈₀ is K, X₈₁ is A, X₈₂ is L, X₈₃ is K, X₈₄ is S, X₈₅ is A, X₈₆ is Q, X₈₇ is T, X₈₈ is S, X₈₉ is L, which are not established at the same time.

43. The antibody or antigen-binding fragment thereof according to claim 37, wherein the antibody comprises:
the heavy chain framework region FRH1, FRH2, FRH3, and FRH4 having sequences shown in SEQ ID NO: 45-48 respectively,
(SEQ ID NO: 45)
X₉₆VQLQQSGPELVRPGASVKX₉₇SCKASGYTFT,
(SEQ ID NO: 46)
WVKQSHGX₉₈X₉₉LEWX₁₀₀G,
(SEQ ID NO: 47)
X₁₀₁X₁₀₂TX₁₀₃TVX₁₀₄X₁₀₅SX₁₀₆STAYMELRSLTSEDSAVYYCVT,
(SEQ ID NO: 48)
WGTGTTVTVSS;
the heavy chain framework region FRH1, FRH2, FRH3, and FRH4 having sequences shown in SEQ ID NO: 53-56 respectively,
(SEQ ID NO: 53)
QVQLQQPGSELVKPGASVKX₁₁₄SCKASGYTFT,
(SEQ ID NO: 54)
WVKQX₁₁₅PGQGLEWX₁₁₆G,
(SEQ ID NO: 55)
X₁₁₇X₁₁₈TX₁₁₉TVDX₁₂₀SSSTX₁₂₁YMQLSSLTSEDSAVYYCAR,
(SEQ ID NO: 56)
WGQGTTLTVSS,
wherein, X₉₆ is E or Q, X₉₇ is M or V, X₉₈ is K or Q, X₉₉ is S or R, X₁₀₀ is I or M, X₁₀₁ is K or R, X₁₀₂ is A or V, X₁₀₃ is L or I, X₁₀₄ is N or D, X₁₀₅ is K or T, X₁₀₆ is S or A, X₁₁₄ is L or V, X₁₁₅ is R or A, X₁₁₆ is I or M, X₁₁₇ is R or K, X₁₁₈ is A or V, X₁₁₉ is L or M, X₁₂₀ is K or T, X₁₂₁ is A or V.

44. The antibody or antigen-binding fragment thereof according to claim 37, wherein the antibody has a light chain variable region with an amino acid sequence shown in SEQ ID NO: 57-59 or SEQ ID NO: 65-67; or
the antibody has a heavy chain variable region with an amino acid sequence shown in SEQ ID NO: 60-64 or SEQ ID NO: 68-72.

45. The antibody or antigen-binding fragment thereof according to claim 37, wherein the antibody comprises at least one of a heavy chain constant region and a light chain constant region, and at least a part of at least one of the heavy chain constant region and the light chain constant region is derived from at least one of a human antibody;
optionally, both the light chain constant region and the heavy chain constant region of the antibody are derived from a human IgG antibody or a mutant thereof;
optionally, the light chain constant region of the antibody is derived from light chain constant region of human Kappa; the heavy chain constant region of the antibody is derived from human IgG4 or IgG1, or a mutant of IgG4 or IgG1.

46. The antibody or antigen-binding fragment thereof according to claim 37, wherein the full-length sequence of the antibody constant region is shown in SEQ ID NO: 73, 74 or 75.

47. The antibody or antigen-binding fragment thereof according to claim 37, wherein the antibody has a light chain as shown in any one of SEQ ID NO: 76-78 and a heavy chain as shown in any one of SEQ ID NO: 79-88; or
the antibody has a light chain as shown in any one of SEQ ID NO: 89-91 and a heavy chain as shown in any one of SEQ ID NO: 92-101.

48. The antibody or antigen-binding fragment thereof according to claim 37, wherein the antibody comprises:
the light chain as shown in SEQ ID NO: 76, the heavy chain as shown in SEQ ID NO: 79, or
the light chain as shown in SEQ ID NO: 77, the heavy chain as shown in SEQ ID NO: 79, or
the light chain as shown in SEQ ID NO: 76, the heavy chain as shown in SEQ ID NO: 80, or
the light chain as shown in SEQ ID NO: 77, the heavy chain as shown in SEQ ID NO: 80, or
the light chain as shown in SEQ ID NO: 76, the heavy chain as shown in SEQ ID NO: 81, or
the light chain as shown in SEQ ID NO: 77, the heavy chain as shown in SEQ ID NO: 81, or
the light chain as shown in SEQ ID NO: 78, the heavy chain as shown in SEQ ID NO: 81, or
the light chain as shown in SEQ ID NO: 77, the heavy chain as shown in SEQ ID NO: 82, or
the light chain as shown in SEQ ID NO: 78, the heavy chain as shown in SEQ ID NO: 82, or
the light chain as shown in SEQ ID NO: 77, the heavy chain as shown in SEQ ID NO: 83, or
the light chain as shown in SEQ ID NO: 78, the heavy chain as shown in SEQ ID NO: 83, or
the light chain as shown in SEQ ID NO: 76, the heavy chain as shown in SEQ ID NO: 84, or
the light chain as shown in SEQ ID NO: 77, the heavy chain as shown in SEQ ID NO: 84, or
the light chain as shown in SEQ ID NO: 76, the heavy chain as shown in SEQ ID NO: 85, or
the light chain as shown in SEQ ID NO: 77, the heavy chain as shown in SEQ ID NO: 85, or
the light chain as shown in SEQ ID NO: 76, the heavy chain as shown in SEQ ID NO: 86, or
the light chain as shown in SEQ ID NO: 77, the heavy chain as shown in SEQ ID NO: 86, or
the light chain as shown in SEQ ID NO: 78, the heavy chain as shown in SEQ ID NO: 86, or
the light chain as shown in SEQ ID NO: 77, the heavy chain as shown in SEQ ID NO: 87, or
the light chain as shown in SEQ ID NO: 78, the heavy chain as shown in SEQ ID NO: 87, or
the light chain as shown in SEQ ID NO: 77, the heavy chain as shown in SEQ ID NO: 88, or
the light chain as shown in SEQ ID NO: 78, the heavy chain as shown in SEQ ID NO: 88.

49. The antibody or antigen-binding fragment thereof according to claim 37, wherein the antibody comprises:
the light chain as shown in SEQ ID NO: 89, the heavy chain as shown in SEQ ID NO: 92, or
the light chain as shown in SEQ ID NO: 90, the heavy chain as shown in SEQ ID NO: 92, or
the light chain as shown in SEQ ID NO: 89, the heavy chain as shown in SEQ ID NO: 93, or
the light chain as shown in SEQ ID NO: 90, the heavy chain as shown in SEQ ID NO: 93, or
the light chain as shown in SEQ ID NO: 89, the heavy chain as shown in SEQ ID NO: 94, or
the light chain as shown in SEQ ID NO: 90, the heavy chain as shown in SEQ ID NO: 94, or
the light chain as shown in SEQ ID NO: 91, the heavy chain as shown in SEQ ID NO: 94, or
the light chain as shown in SEQ ID NO: 90, the heavy chain as shown in SEQ ID NO: 95, or
the light chain as shown in SEQ ID NO: 91, the heavy chain as shown in SEQ ID NO: 95, or
the light chain as shown in SEQ ID NO: 90, the heavy chain as shown in SEQ ID NO: 96, or
the light chain as shown in SEQ ID NO: 91, the heavy chain as shown in SEQ ID NO: 96, or
the light chain as shown in SEQ ID NO: 89, the heavy chain as shown in SEQ ID NO: 97, or
the light chain as shown in SEQ ID NO: 90, the heavy chain as shown in SEQ ID NO: 97, or
the light chain as shown in SEQ ID NO: 89, the heavy chain as shown in SEQ ID NO: 98, or
the light chain as shown in SEQ ID NO: 90, the heavy chain as shown in SEQ ID NO: 98, or
the light chain as shown in SEQ ID NO: 89, the heavy chain as shown in SEQ ID NO: 99, or
the light chain as shown in SEQ ID NO: 90, the heavy chain as shown in SEQ ID NO: 99, or
the light chain as shown in SEQ ID NO: 91, the heavy chain as shown in SEQ ID NO: 99, or
the light chain as shown in SEQ ID NO: 90, the heavy chain as shown in SEQ ID NO: 100, or
the light chain as shown in SEQ ID NO: 91, the heavy chain as shown in SEQ ID NO: 100, or
the light chain as shown in SEQ ID NO: 90, the heavy chain as shown in SEQ ID NO: 101, or
the light chain as shown in SEQ ID NO: 91, the heavy chain as shown in SEQ ID NO: 101.

50. A CAR-T cell, wherein the CAR-T cell comprises a chimeric antigen receptor, wherein the chimeric antigen receptor includes:
an extracellular region, wherein the extracellular region includes a heavy chain variable region and a light chain variable region of a single-chain antibody, wherein the light chain variable region has an amino acid sequence as shown in SEQ ID NO: 57-59 or SEQ ID NO: 65-67, and the heavy chain variable region has an amino acid sequence as shown in SEQ ID NO: 60-64 or SEQ ID NO: 68-72;
a transmembrane region, wherein the transmembrane region is connected to the extracellular region and embedded in the cell membrane of the T lymphocyte; and
an intracellular region, wherein the intracellular region is connected to the transmembrane region.

51. The CAR-T cell according to claim 50, wherein the transmembrane region is an ICOS transmembrane region, an CD8 transmembrane region or a OX40 transmembrane region;
preferably, the transmembrane region is a CD8 transmembrane region;
more preferably, the amino acid sequence of the transmembrane region is shown in SEQ ID NO: 106.

52. The CAR-T cell according to claim 50, wherein the intracellular segment is a 4-1BB intracellular segment and an ICOS or OX-40 intracellular segment;
preferably, the intracellular segment is a 4-1BB intracellular segment and an ICOS intracellular segment;
more preferably, the amino acid sequence of the 4-1BB intracellular segment is shown in SEQ ID NO: 109; the amino acid sequence of the ICOS intracellular segment is shown in SEQ ID NO: 110.

53. The CAR-T cell according to claim 50, wherein the extracellular region further comprises a CD8 hinge region;
preferably, the amino acid sequence of the CD8 hinge region is shown in SEQ ID NO: 111.

54. The CAR-T cell according to claim 50, wherein the intracellular segment further comprises a CD3ζ chain;
preferably, the amino acid sequence of the CD3ζ chain is shown in SEQ ID NO: 112.

55. The CAR-T cell according to claim 53, wherein the N-terminus of the intracellular segment of ICOS is connected to the C-terminus of the CD8 transmembrane region, the C-terminus of the intracellular segment of ICOS is connected to the N-terminus of the intracellular segment of 4-1BB, and the C-terminus of the intracellular segment of 4-1BB is connected to the N-terminus of the CD3ζ chain.

56. A method for treating or preventing Claudin18.2 related diseases, wherein the method comprising administering to a subject the antibody of claim 37;
optionally, the Claudin18.2 related diseases include: at least one of gastric cancer, esophageal cancer, pancreatic cancer, lung cancer, colon cancer and rectal cancer.
